(19) United States
(12) Patent Application Publication
(10) Pub. No.: US 2004/0014086 A1
(43) Pub. Date: Jan. 22, 2004

(54) Regulome arrays

(75) Inventors: John A. Stamatoyannapoulos, Boston, MA (US); Michael McArthur, Rockland (GB); Michael Hawrylycz, Seattle, WA (US)

Correspondence Address: Pennie & Edmonds LLP, 1155 Avenue of the Americas, New York, NY, 10036-2711, US

(73) Assignee: Regulome Corporation, Seattle, WA

(21) Appl. No.: 10/375,404

(22) Filed: Feb. 27, 2003

Related U.S. Application Data
(63) Continuation-in-part of application No. 10/319,440, filed on Dec. 12, 2002.
(63) Continuation-in-part of application No. PCT/US02/15032, filed on May 13, 2002.
(60) Provisional application No. 60/290,036, filed on May 11, 2001.
(60) Provisional application No. 60/378,664, filed on May 9, 2002.

(51) Int. Cl. C12Q 1/68

(52) U.S. Cl. 435/6

(57) ABSTRACT

Arrays, probes and methods are disclosed for the construction and interrogation of DNA arrays containing genomic functional sites, and thereby active genetic regulatory sequences. Further methods are disclosed for interrogation of such arrays in order to reveal the pattern of genetic functional and regulatory activity within any given cell(s) or tissue type(s) or associated with any particular genetic locus or combination of loci under a variety of conditions.

This application is a continuation-in-part application of U.S. application Ser. No. 10/319,440, filed Dec. 12, 2002, and a continuation-in-part application of International Application No. PCT/US02/15032, filed May 12, 2002, which claims benefit of U.S. Provisional Application No. 60/290,036, each of which is incorporated by reference herein in its entirety.

### FIELD OF THE INVENTION

The invention relates to DNA arrays for simultaneous detection of genomic functional sites, their manufacture and use. The invention further concerns array methods, devices, systems, and algorithms for detecting patterns of genomic functional sites active or inactive in eukaryotic cells, and particularly chromatin elements and genetic control elements active in eukaryotic cells.

### BACKGROUND OF THE INVENTION

Conventional gene expression studies generally employ immobilized DNA molecules that are complementary to gene transcripts (either the entire transcript or to selected regions thereof) that are transcribed and spliced into mRNA. Recent advances in this field utilize arrays or microarrays of such molecules that enable simultaneous monitoring of multiple distinct transcripts (see, e.g., Schena et al., Science 270:467-470 (1995); Lockhart et al., Nature Biotechnology 14:1675-1680 (1996); Blanchard et al., Nature Biotechnology 14, 1649 (1996); and U.S. Pat. No. 5,569,588, issued Oct. 29, 1996 to Ashby et al. entitled “Methods for Drug Screening.”). Such arrays have the potential to detect transcripts from virtually all actively transcribed regions of a cell or cell population, provided the availability of an organism's complete genomic sequence, or at least a sequence or library comprising all of its gene transcripts. In the case of the Human where a complete gene set remains unclear, such arrays may be employed to monitor simultaneously large numbers of expressed genes within a given cell population.

The simultaneous monitoring technologies particularly relate to identifying genes implicated in disease and in identifying drug targets (see, e.g., U.S. Pat. Nos. 6,165,709; 6,218,122; 5,811,231; 6,203,987; and 5,569,588). Unfortunately, these array technologies generally rely on direct detection of expressed genes and therefore reveal only indirectly the activity of genetic regulatory pathways that control gene expression itself. On the other hand, a detection system directed toward sensing the activity of particular genetic regulatory pathways or cis-acting regulatory elements could provide deeper information concerning a cell's regulatory state. Accordingly, the detection of active regulatory elements, particularly in related and interacting groups, potentially could become extremely important for delineation of regulatory pathways, and provide critical knowledge for design and discovery of disease diagnostics and therapeutics.

Most research in the area of gene regulation has focused on finding and using individual sequences either upstream or downstream of individual coding gene targets. Generally, the presence of absence of a particular DNA sequence is linked with increased or decreased expression of a nearby gene when determining the regulatory effect of the sequence. For example, the beta-like globin gene was shown to contain four major DNAase I hypersensitive sites of possible regulatory function by studies that removed or added these sequences and that looked for an effect on gene expression in erythroid cells. See Grosveld et. al. U.S. Pat. No. 5,532,143. From related studies, Townes et al. asserted that two of the four DNAse hypersensitive sites might control genes generally in cells of erythroid lineage. Although an interesting development, these observations generally are limited to detection of effects on nearby coding sequences of known genes. Multiple regulatory units, which behave coordinately, are not readily amenable to analysis by these techniques.

Multiple gene and protein elements interact for even simple biological processes. Because of this, a one at a time strategy for targeting a single coding gene and nearby non-coding sequences to determine their effects on the preselected gene insufficiently addresses the true in vivo situation. Accordingly, any tool that can provide simultaneous regulation system information would give rich benefits in terms of improved diagnosis, clinical treatment and drug discovery.

### SUMMARY OF THE INVENTION

The present invention overcomes the problems and disadvantages associated with current strategies and designs with methods and materials that enable the use of nucleic acid arrays for profiling large numbers of functional sites, and hence active genetic regulatory units.

One embodiment of the invention is directed to methods for manufacturing an array of functional sites. Since virtually all active genomic regulatory regions are contained within functional sites, an array of functional sites constitutes an array of regulatory elements. Generally, a nucleic acid microarray is made having spots that contain copies of sequences corresponding to a genomic DNA sequence that contains a functional site or a putative genomic regulatory element. In certain illustrative embodiments, the nucleic acid sequences are obtained by amplifying sequences from a library, e.g., a library of functional sites as described herein, using the polymerase chain reaction, and depositing material with a microarraying apparatus, or synthesizing ex situ using an oligonucleotide synthesis device, and subsequently depositing using a microarraying apparatus, or synthesizing in situ on the microarray using a method such as piezoelectric deposition of nucleotides.

Another embodiment of the invention is directed to methods for analyzing functional sites comprising: preparing chromatin from a target cell population; treating said chromatin with an agent that induces modifications at functional sites in chromatin, such as a non-specific restriction endonuclease, to induce single and double stranded cleavage at such locations in marked preference to other locations within the genome; modifying the fragment ends through the ligation of a linker adapter or similar means to tag the sequences in a manner such that they can be separated from the mixture; modifying the fragments to reduce the average fragment size by digest with a restriction enzyme or by sonication or an equivalent procedure; labeling the fragment subpopulation containing functional site sequences with a fluorescent dye or other marker sufficient for detection through an automated apparatus such as a DNA microarray reader; incubating the labeled fragment population with a microarray according to the present invention and recording the signal intensity at each array coordinate. In this way, one can effectively and efficiently identify one or more functional sites present in or associated with, e.g., active within, the sample from which the labeled fragment population was derived.

Yet another embodiment of the invention is a procedure for profiling functional sites from a cell or organism, comprising a first step of constructing a DNA microarray that contains functional sites, and a second step of probing the microarray to assay the presence of functional sites. The first step involves constructing a DNA microarray having spots with one or more copies of a DNA sequence corresponding to a genomic DNA sequence that contains a nuclease functional site or a putative genomic regulatory element. The DNA sequences contained on the array may be obtained or deposited alternative ways, for example: by amplifying the DNA sequences using PCR from a library, such as a functional site library containing such sequences and subsequently depositing with a microarraying apparatus; synthesizing the DNA sequences ex situ with an oligonucleotide synthesis device and subsequently depositing with a microarraying apparatus; or by synthesizing the DNA sequences in situ on the microarray by, for example, piezoelectric deposition of nucleotides. The number of sequences deposited on the array may vary between 10 and several million depending on the technology employed to create the array.

In another embodiment of the invention, a DNA microarray containing genomic DNA sequences corresponding to established or putative functional site or regulatory elements is assayed in five steps. In step one, chromatin from a sample, e.g. cell, is prepared and treated with an agent that induces modifications at functional sites. For example, the non-specific restriction endonuclease DNAse I may be used to induce single and double stranded cleavage at such locations in marked preference to other locations within the genome. Secondly, the fragment ends are modified through the ligation of a linker adapter, enzymatic labeling or similar means to tag the sequences in a manner such that they can be separated from the mixture. Thirdly, the DNA fragments may be modified further to reduce the average fragment size by digest with a restriction enzyme, by sonication or an equivalent procedure. Fourthly, the DNA fragment subpopulation containing functional site sequences is labeled with a fluorescent dye or other marker sufficient for detection through an automated apparatus such as a DNA microarray reader. A last step is incubation of the labeled fragment population with a DNA microarray according to the present invention and recording the signal intensity at each array coordinate.

According to another aspect of the invention, there is provided a method of ascertaining the effect of a test compound, e.g., a chemical agent, biological agent or other environmental perturbation, on a functional site or regulatory profile of a tissue obtained from a eukaryotic organism. The method generally involves obtaining a first profile for binding between functional sites isolated from of the tissue that is unexposed to the test compound or perturbation and a microarray according to the present invention. A second profile is obtained for binding between functional sites of the tissue that is exposed to the test compound or perturbation and a microarray according to the invention. By comparing the first profile with the second profile, the functional sites that are effected by the perturbation are thereby revealed. Contact with a test compound or perturbation may occur before obtaining the tissue from the organism and may be selected from the illustrative group consisting of an infection of the eukaryotic organism from a microorganism, loss in immune function of the eukaryotic organism, exposure of the tissue to high temperature, exposure of the tissue to low temperature, cancer of the tissue, cancer of another tissue in the eukaryotic organism, irradiation of the tissue, exposure of the tissue to a chemical or other pharmaceutical compound; and aging. Alternatively, contact with a test compound or perturbation may occur after obtaining the tissue from the organism and may be selected from the illustrative group consisting of exposure of the tissue to high temperature, exposure of the tissue to low temperature, irradiation of the tissue, exposure of the tissue to a chemical or other pharmaceutical compound, and aging.

According to another aspect of the invention, there is provided a method of discerning at least one set of co-regulated genes in cells of a eukaryotic organism, comprising obtaining a first profile for binding between functional sites of the tissue under controlled culture conditions; obtaining a second profile for binding between functional sites of the tissue under conditions where a known regulator of at least one of the genes is altered with respect to the controlled culture conditions; and comparing the first profile with the second profile to determine which functional sites are effected by the alteration of the known regulator. Illustrative regulators include hormones, nutrients, pharmacologically active chemicals, and the like.

According to another aspect of the invention, there is provided a method for profiling differential functional sites present in or isolated from two populations that contain nucleic acid. This generally involves first obtaining multiple functional sites from a first population and labeling them with a first label and obtaining multiple functional sites from a second population and labeling them with a second label. The functional sites are then hybridized with a DNA microarray of the present invention, preferably containing DNA species in separate locations that match putative or verified regulatory elements, in order to determine the ratio of signals from the first and second labels within the array. This allows for the rapid and efficient identification of differences in functional site presence between two or more sample populations. In one example, one of the populations is an untreated control and the other population is treated by contact with at least one test compound or other perturbation, and the signal ratios obtained provide an indication of gene regulatory activity by the at least one test compound or perturbation.

According to another aspect of the invention, there is provided a method of identifying a functional site profile associated with a disease state, such as cancer, comprising obtaining a first profile or set of profiles for binding between functional sites of a tissue and an array of the invention, said first profile or set of profiles being representative of a normal healthy condition. A second profile or set of profiles is also obtained for binding between functional sites of a tissue and an array of the invention, said second profile or set of profiles being representative of a disease condition. By comparing the first profile or set of profiles with the second profile or set of profiles, one can readily identify alterations in the presence or activity of one or more functional sites in the disease condition relative to the normal condition. The invention thus further encompasses a disease associated functional site profile or set of profiles identified according to the above method, as well as methods for diagnosing the presence of a disease condition in a patient, comprising obtaining a functional site profile for a biological sample obtained from a patient suspected of having said disease condition and comparing said functional site profile to a disease-associated functional site profile.

In another aspect, the invention provides methods of preparing probes that may be used according to methods of the invention, including methods of screening arrays and methods of profiling cells and functional sites.

In one embodiment, the invention provides a method of preparing fixed length direct monotagged nucleic acids that includes treating genomic DNA with an agent that cleaves DNA, ligating the treated genomic DNA with a blunt or T-tailed linker containing a type IIs restriction endonuclease restriction site, and treating the ligated DNA with a type IIs restriction enzyme. In one particular embodiment, the cleavage is performed using DNase I in the presence of manganese. In a related embodiment, the agent that cleaves DNA is a restriction endonuclease.

In another embodiment, the invention provides a method of preparing fixed length indirect monotagged nucleic acids that includes treating genomic DNA with an agent that cleaves DNA, capturing the treated genomic DNA, treating the captured genomic DNA with a restriction enzyme, ligating the DNA with a linker comprising a type IIs restriction enzyme site, and treating the ligated DNA with a type II restriction enzyme. In one particular embodiment, the cleavage sites within the genomic DNA are captured following biotinylation or ligation of a biotinylated linker.

A related embodiment of the invention provides a method of profiling functional sites in a cell, comprising preparing fixed length direct monotagged or fixed length indirect monotagged nucleic acids according to the invention and hybridizing the genomic DNA to an array comprising functional sites. Such methods may further comprise an identification step, such as, for example, detecting hybridized or bound nucleic acids.

Another related embodiment provides method of profiling a cell, comprising preparing genomic DNA according to a method of the invention and hybridizing the genomic DNA to an array comprising a plurality of DNA sequences. This method may also further comprise an identification step, such as, for example, detecting hybridized or bound nucleic acids. Other embodiments and advantages of the invention are set forth in part in the description which follows, and in part, will be obvious from this description, or may be learned from practice of the invention.

The present invention provides methods of profiling the genomic regulatory regions of a biological sample, comprising: (a) contacting a sample of nucleic acid from a biological sample, with a positionally addressable array of polynucleotides under conditions such that hybridization can occur, said sample of nucleic acid being enriched in ACEs or fragments thereof of at least 10 base pairs; and (b) detecting loci on the array where hybridization occurs, wherein said ACEs are each a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and wherein said array of polynucleotides comprises a plurality of polynucleotides, each affixed to a substrate, said plurality comprising different polynucleotides differing in nucleotide sequence and being situated at distinct loci of the array, said different polynucleotides being complementary and hybridizable to genomic DNA of said biological sample, thereby profiling the genomic regulatory regions of the biological sample. In certain embodiments, the methods of profiling the genomic regulatory regions of a biological sample further comprise measuring the amount of hybridization at each said loci. In other embodiment, the methods of profiling the genomic regulatory regions of a biological sample further comprise, prior to step (a), a step of enriching the sample of nucleic acid in ACEs. In one embodiment, a method of enriching a sample of nucleic acid in ACEs comprises: (a) contacting the chromatin sample with a nucleic acid modifying agent, thereby producing a modified chromatin sample; (b) subjecting the modified genomic chromatin to size fractionation, thereby producing a plurality of modified chromatin fractions; (c) isolating one or more modified chromatin fractions corresponding to DNA of greater than 100 nucleotides in length, thereby enriching the chromatin sample for genomic regulatory regions.

The present invention further provides positionally addressable polynucleotide arrays comprising ACEs an/or suitable for probing for ACEs. The arrays can be solid phase arrays or semi-solid phase arrays.

In certain embodiments, the present invention provides a positionally addressable polynucleotide array comprising a plurality of different polynucleotides, each different polynucleotide (a) differing in nucleotide sequence, (b) being affixed to a substrate at a different locus, (c) being in the range of 10-1000 nucleotides in length, and (d) being complementary and hybridizable to a predetermined ACE, each said ACE being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and wherein the loci at which said different polynucleotides are situated are at least 15% of the total loci of the array. In one embodiment, each different polynucleotide is greater than 30 nucleotides and is designed so as not to contain a sequence of in the range of 15-30 nucleotides that occurs in the genome of the organism from which the ACEs are identified greater than 10 times. In one mode of the embodiment, desigining each said different polynucleotide is performed by a method comprising (a) identifying by comparing to an indexed polynucleotide set a sequence in said different polynucleotide, wherein said sequence consists of a nucleotide sequence in the range of 10-15 nucleotides and has a frequency count less than 11 in the genome of said organism, and wherein said indexed polynucleotide set contains binary encoded nucleotide sequences of sizes in the range of 10-15 nucleotides; (b) determining the genomic locations of said sequence from said indexed polynucleotide set; (c) adding prefix and suffix nucleotide sequences to said sequence according to the genomic sequence at each of said genomic locations to generate a set of candidate polynucleotides; and (d) accepting a polynucleotide from said set of candidate polynucleotides if the respective alignment of the sequences of its added prefix and suffix sequences and the prefix and suffix sequences of said sequence in the corresponding predetermined ACE is above a given threshold.

The present invention further provides positionally addressable polynucleotide arrays to which nucleic acids are hybridized, in which the polynucleotides affixed to the array and/or the nucleic acids hybridized to the array are enriched in ACE sequences. Such arrays can be solid phase arrays or semi-solid phase arrays.

In certain embodiments, the present invention provides a positionally addressable polynucleotide array to which nucleic acids are hybridized, said array comprising a plurality of different polynucleotides, each different polynucleotide (a) differing in nucleotide sequence and (b) being affixed at a different locus to a substrate, said nucleic acids being enriched in ACEs or fragments thereof of at least 10 base pairs, each said ACE being a nucleotide sequence characterized as being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, said nucleic acids being hybridized to one or more discrete loci on the array.

In other embodiments, the present invention provides a positionally addressable polynucleotide array to which nucleic acids are hybridized, said array comprising a plurality of different polynucleotides, each different polynucleotide (a) differing in nucleotide sequence, (b) being affixed at a different locus to a substrate, (c) being in the range of 10-1000 nucleotides in length, and (d) being complementary and hybridizable to a predetermined ACE, each said ACE being a nucleotide sequence characterized as being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and wherein the loci at which said different polynucleotides are situated are at least 15% of the total loci of the array.

In other embodiments, the present invention provides a positionally addressable polynucleotide array to which nucleic acids are hybridized, said array comprising a plurality of different polynucleotides, each different polynucleotide (a) differing in nucleotide sequence, (b) being affixed at a different locus to a substrate, (c) being in the range of 10-1000 nucleotides in length, and (d) being complementary and hybridizable to a predetermined ACE, each said ACE being a nucleotide sequence characterized as said ACE being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, wherein the loci at which said different polynucleotides are situated are at least 15% of the total loci of the array; and wherein said nucleic acids are enriched in ACEs or fragments thereof of at least 10 base pairs.

The present invention yet further provides methods of identifying one or more genomic regulatory regions involved in a cellular response to a perturbation, comprising: (a) comparing a profile of a plurality of ACEs of cells exposed to a perturbation with a profile of a plurality of ACEs of cells of the same cell type not exposed to the perturbation, wherein each said ACE is a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, (b) identifying one or more ACEs that are detected to a greater or lesser extent in the cells exposed to the perturbation relative to the cells not exposed to the perturbation, thereby identifying one or more genomic regulatory regions involved in a cellular response to the perturbation.

A comparison of ACE profiles can be preceded by obtaining a profile of ACEs of the cells exposed to the perturbation and/or obtaining a profile of ACEs of the cells not exposed to the perturbation. Obtaining a profile of the cells exposed to the perturbation can be performed by a method comprising: (i) contacting a sample of nucleic acid from the cells exposed to the perturbation, said sample of nucleic acid being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of said cells exposed to the perturbation, under conditions such that hybridization can occur; and (ii) detecting loci on the array where hybridization occurs. Obtaining a profile of the cells not exposed to the perturbation can be performed by a method comprising: (i) contacting a sample of nucleic acid from the cells not exposed to the perturbation, said sample of nucleic acid being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of said cells not exposed to the perturbation, under conditions such that hybridization can occur; and (ii) detecting loci on the array where hybridization occurs.

The present invention yet further provides methods of deducing a regulatory network, comprising: (a) identifying at least two ACEs involved in a cellular response to a perturbation, for example as described above, (b) identifying at least two genes in which any of the identified ACEs are contained, thereby deducing a regulatory network comprising said identified genes.

The present invention yet further provides methods of identifying one or more disease-associated regulatory regions, comprising: (a) comparing a profile of a plurality of ACEs of diseased cells with the profile of a plurality of ACEs of control cells of the same cell type as the diseased cell, wherein each said ACE is a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, (b) identifying one or more ACEs that are detected to a greater or lesser extent in the diseased cells relative to the control cells, thereby identifying one or more disease-associated regulatory regions.

A comparison of ACE profiles can be preceded by obtaining a profile of ACEs of the diseased cells and/or obtaining a profile of ACEs of the control cells. Obtaining a profile of the diseased cells can be performed by a method comprising: (i) contacting a sample of nucleic acid from the diseased cells, said sample of nucleic acid being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of said diseased cells, under conditions such that hybridization can occur; and (ii)

detecting loci on the array where hybridization occurs. Obtaining a profile of the control cells can be performed by a method comprising: (i) contacting a sample of nucleic acid from the control cells, said sample of nucleic acid being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of said control cells, under conditions such that hybridization can occur; and (ii) detecting loci on the array where hybridization occurs.

The present invention yet further provides methods of identifying one or more disease-associated genes, comprising: (a) identifying one or more disease-associated ACEs, for example as described above; and (b) identifying the genes in which any of the identified ACEs are contained, thereby identifying one or more disease-associated genes.

The present invention yet further provides methods of diagnosis, prognosis, staging or monitoring therapy of a disease in a patient, comprising: (a) comparing the detection of one or more ACEs in a nucleic acid sample from a patient with the detection of one or more ACEs in a control nucleic acid sample, wherein each said ACE is a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, (b) identifying one or more ACEs that are detected to a greater or lesser extent in the nucleic acid sample from the patient relative to the control nucleic acid sample, thereby diagnosing, prognosing, staging or monitoring therapy of a disease in a patient. Detection of one or more ACEs in the nucleic acid sample from the patient can be performed by a method comprising: (i) contacting said nucleic acid from the patient, said nucleic acid being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of the patient, under conditions such that hybridization can occur; and (ii) detecting loci on the array where hybridization occurs, thereby detecting one or more ACEs in the nucleic acid sample from the patient. Optionally, prior to step (i), the nucleic acid from the patient can be enriched in ACEs.

In the foregoing diagnostic, prognostic, staging or monitoring methods, detection of one or more ACEs in the control sample is performed by a method comprising: (i) contacting nucleic acid from the control sample, said nucleic acid from the control sample being enriched in ACEs or fragments thereof of at least 10 base pairs, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA of said control sample, under conditions such that hybridization can occur; and (ii) detecting loci on the array where hybridization occurs, thereby detecting one or more ACEs in the control sample.

In certain embodiments of the foregoing diagnostic, prognostic, staging or monitoring methods, the control nucleic acid sample is from cells (i) having said disease, and (ii) of the same cell type as the cell type from which the nucleic acid sample from the patient is isolated. In other embodiments, the control nucleic acid sample is from cells (i) not having said disease, and (ii) of the same cell type as the cell type from which the nucleic acid sample from the patient is isolated.

In a method of monitoring therapy according to the present invention, the control nucleic acid sample can be from cells removed from the patient at an earlier time point than the time point at which the cells from which the nucleic acid sample (being monitored) from the patient is isolated are removed from said patient.

In a method of prognosis according to the present invention, the control nucleic acid sample can be from diseased cells of a predetermined stage of disease.

The present invention yet further provides methods for identifying the active gene regulatory sequences bound by a transcription factor comprising: (a) subjecting the nucleoprotein of a cell to a protein cross-linking agent, thereby producing cross-linked nucleoprotein; (b) subjecting the cross-linked nucleoprotein to immunoprecipitation using an antibody that immunospecifically binds to a transcription factor, thereby producing a cross-linked immunoprecipitate; (c) recovering the DNA present in the cross-linked immunoprecipitate, thereby producing recovered DNA; and (d) identifying the recovered DNA by a method comprising: (i) contacting the recovered DNA with a positionally addressable array of polynucleotides, each different polynucleotide (1) differing in nucleotide sequence, (2) being affixed at a different locus to a substrate, (3) being in the range of 10-1000 nucleotides in length, and (4) being complementary and hybridizable to a predetermined ACE, each said ACE being a nucleotide sequence characterized as being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and wherein the loci at which said different polynucleotides are situated at least 15% of the total loci of the array, under conditions such that hybridization can occur; and (ii) detecting loci on the array where hybridization occurs, thereby identifying the active gene regulatory sequences bound by the transcription factor.

The present invention yet further provides methods of determining whether an aberrant copy number of a genomic sequence is present in a test biological sample, comprising determining whether one or more ACEs are detected to a greater or lesser extent in a first sample of genomic DNA, or nucleic acid derived therefrom, said first sample of genomic DNA being from the test biological sample, relative to the detection of said one or more ACEs in a second genomic DNA sample, or nucleic acid derived therefrom, said second sample of genomic DNA being from a control biological sample having a known copy number of said one or more ACEs, wherein said ACE is a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, thereby determining whether an aberrant copy number of a genomic sequence is present in the test biological sample. In certain embodiment, said determining whether one ore more ACEs are detected to a greater or lesser extent in said first sample of genomic DNA or nucleic acid derived therefrom, relative to the detection of said one or more ACEs in said second sample of genomic DNA, or nucleic acid derived therefrom, comprises: (a) contacting nucleic acid enriched in ACEs or fragments thereof of at least 10 base pairs from (i) said first sample of genomic DNA or (ii) nucleic acid derived therefrom, with a positionally addressable array of polynucleotides, in which said array of polynucleotides comprises a plurality of polynucleotides, each affixed to a substrate, said plurality of polynucleotides (1) differing in nucleotide sequence, (2) comprising different polynucleotides situated at distinct loci of the array, and (3) and being complementary and hybridizable to predetermined genomic DNA in the first sample of genomic DNA, under conditions such that hybridization can occur; (b) detecting one or more loci on the array where hybridization occurs; (c) comparing the signal at said one or more loci of step (b) with signal generated by performing steps (a)-(b) with said (i) second sample of genomic DNA or (ii) nucleic acid derived therefrom; thereby determining whether one ore more ACEs are detected to a greater or lesser in extent in said first sample of genomic DNA or nucleic acid derived therefrom, relative to the detection of said one or more ACEs in said second sample of genomic DNA, or nucleic acid derived therefrom.

In the foregoing methods and compositions, the The ACEs can further be characterized as having one, two, three or more of the following characteristics: (i) an intrinsic ability to confer hypersensitivity to the DNA modifying agent when excised from its native location and inserted into at least one different location in the genome of a cell of the same cell type; (ii) 10-50 times greater hypersensitivity to the DNA modifying agent relative to the nearby region; (iii) 50-100 times greater hypersensitivity to the DNA modifying agent relative to the nearby region; (iv) 100-150 times greater hypersensitivity to the DNA modifying agent relative to the nearby region; (v) 150-200 times greater hypersensitivity to the DNA modifying agent relative to the nearby region; (vi) the ability to reconstitute a site that is hypersensitive to the DNA modifying agent when a nucleic acid comprising the nucleotide sequence flanked by at least 1000 bp on each side is assembled into chromatin in an in vitro reconstitution assay in the presence of nucleosomal proteins and a cell extract; (vii) is non-nucleosomal when present in chromatin isolated from one or more cells; (viii) is embedded in DNA associated with histones that have a high degree of acetylation when present in chromatin isolated from one or more cells; (ix) greater solubility than nucleosomal material in moderate salt solutions (e.g., 150 mM NaCl and 3 mM MgCl2) when present in chromatin isolated from one or more cells; (x) is a non-coding sequence; or (xi) does not occur greater than 10 times in a genome of the organism in which the ACE is identified.

In various embodiments of the foregoing methods and compositions, ACEs or fragments thereof represent at least 30%, 40%, 50%, 60%, 70%, 80%, or 90% of the total nucleic acid in a sample of nucleic acid enriched in ACEs. In a certain specific embodiments, a sample of nucleic acid enriched in ACEs is enriched in ACEs to the degree of purity, such that ACEs or fragments represent at least 95%, at least 98%, or at least 99% of the total nucleic acid in the sample of nucleic acid.

In other various embodiments of the foregoing methods and compositions, polynucleotides comprising ACE sequences or fragments thereof of at least 15, 20, 30 or 40 nucleotides represent at least 15%, 20%, 25%, 30%, 40%, 50%, 60%, 70%, 80%, or 90% of the polynucleotides on a positionally addressable polynucleotide array. Further, in various embodiments, the plurality of polynucleotides on a positionally addressible array is at least 100, at least 200, at least 300, at least 400, at least 500, at least 600, at least 800, at least 1,000, at least 5,000, at least 10,000 or at least 20,000 different polynucleotides.

A profile of ACEs of cells comprises is preferably at least 3 different ACEs, is more preferably at least 5 different ACEs, is more preferably at least 10 different ACEs, is more preferably at least 20 different ACEs, and yet is more preferably at least 50 different ACEs. In various embodiments, a profile of ACEs it at least 100, at least 200, at least 500, or at least 1000 different ACEs.

Biological samples assayed or profiled by the methods of the present invention can include cell culture samples or a primary tissue sample (e.g., a tissue biopsy).

### DESCRIPTION OF THE FIGURES

FIG. 1 is an overview of an embodiment for assaying functional site activity using regulome microarrays.

FIG. 2 illustrates an approach for profiling functional site activity using a two-dye system to increase signal-to-noise ratio.

FIG. 3 illustrates an approach for profiling differential functional site representation in two different samples.

FIG. 4 illustrates an approach for the use of functional site arrays to screen drugs and/or small molecule compounds.

FIG. 5 illustrates an approach for identifying a correlation between functional site presence or activity and gene expression obtained by an embodiment of the invention.

FIG. 6 shows the use of an embodiment for controlling quality of conventional expression arrays.

FIG. 7 illustrates a Hash table structure implemented during the indexing phase of MerCator.

FIG. 8 illustrates the retrieval of a minimum frequency 16-mer and subsequent query of the prefix and suffix positions.

FIG. 9 demonstrates the probability of uniqueness of a k-mer as a function of k.

FIG. 10 provides a depiction of exact frequency distribution of 16-22 mers as calculated using the ScanMer indexing system.

FIG. 11 depicts the results of chromatin fractionation by sucrose gradient ultracentrifugation.

FIG. 12 provides a graph showing the strong correlation between ScanMer scores and genomic hybridization signals.

### DETAILED DESCRIPTION OF THE INVENTION

The expression of genes relies upon the coordinated activities of numerous regulatory networks, all of which ultimately exert their influence through functional sites within genomic DNA. This set of functional sites may be referred to as the “regulome.” These functional sites represent the key regulatory regions of genomic DNA and, thus, govern gene expression and all related biological processes, including, e.g., cell proliferation, differentiation, development, and apoptosis. Furthermore, since the vast majority of diseases are polygenic and due to quantitative variation in gene expression/regulation, the vast majority of functional genetic mutations that cause or modulate disease will be found within functional sites of the regulome. The present invention provides novel compositions and methods for characterizing functional sites of genomic DNA. Such compositions and methods allow the identification and characterization of functional sites present within different cells and tissues, including disease cells. The compositions and methods of the invention provide an integrated approach combining molecular, high throughput and bioinformatic and computation methods, which permits genome-wide global analysis of functional sites. Such genome-wide profiling of functional sites has broad applications in cell characterization, and may be applied, e.g., to identify disease genes and regulatory networks, determine the effects of drugs and other agents, and develop unique characteristic markers of cells, including different cell or tissue types, disease cells, and cells treated with different drugs or agents, for example.

The invention, in certain embodiments, provides arrays of functional sites, methods of preparing and labeling probe populations, methods of screening arrays of functional sites, and methods of analyzing generated data. Relatedly, the invention provides methods of identifying or profiling functional sites within cells, as further described infra.

The following definitions are provided to assist in understanding the various embodiments of the invention as described:

A “functional site” is a specific region of genomic DNA (or its nucleotide sequence), which in the context of nuclear chromatin, is associated with a disruption in chromatin structure and is accessible to a DNA-modifying agent, and which is associated with one or more of the following characteristics: (1) bound by one or more DNA-binding proteins; (2) possesses the intrinsic ability to form in ectopic or heterotopic genomic locations or in a position-independent manner; (3) regulates expression of a gene or set of genes; (4) regulates the chromatin structure of a genetic locus; and/or (5) regulates the structure and enzymatic modification of chromatin through recruitment of chromatin modifying enzymes or chromatin remodeling complexes. Functional sites include isolated polynucleotides corresponding to and forming an inseparable and dominant component of functional sites. Functional sites are biologically-bounded by flanking nucleosomes and span the inter-nucleosomal interval, which is approximately 150-250 base pairs in length. A functional site typically contains a core domain of approximately 80-100 base pairs in length, which is required for formation of the functional site in vivo. In addition, a functional site sequence may further contain flanking regions that modulate the activity of the core domain. A functional site may also be referred to herein as an active chromatin element or ACE.

A “functional site variant” is a region of genomic DNA, which differs in sequence as compared to a functional site at the same genomic location. A functional site variant may or may not be a functional site in one or more cells wherein the corresponding functional site is present.

A “chromatin modifying agent” (CMA) is an agent capable of modifying genomic DNA, in the context of nuclear chromatin, in a detectable manner. Examples of DNA-modifying agents and associated modifications include nucleases (non-specific, e.g., DNase I, and sequence-specific, e.g., restriction endonucleases), DNA-binding proteins (modified and non-modified), DNA-modifying enzymes (e.g., methyl transferases, acetylases), DNA-intercalating agents (e.g., bleomycin, topoisomerases), and integrating viruses.

The “regulome” is the complete set of all functional sites present in a species.

A “tissue regulome” is the complete set of all functional sites present in a particular cell or tissue.

A “regulotype” is a set of functional sites present in a particular individual or organism. Thus, a “regulotype” is specific for the particular individual or organism.

A “tissue regulotype” is a set of functional sites present in a particular cell or tissue of a particular individual or organism. Thus, a tissue regulotype is specific for the particular cell or tissue-type.

“Profiling” is identifying the presence or absence of functional sites in a particular cell at one or more particular genomic loci. Depending upon the origin and/or treatment of the cell being profiled, profiling includes, e.g., tissue profiling, disease profiling, drug profiling, and functional mutant profiling. Profiling may be used to determine the pattern of functional site presence or absence specific to a particular cell or tissue, including, e.g., a diseased cell or a cell treated with a drug.

“Locus profiling” is identifying functional sites present in a particular cell at a particular genomic locus.

A “gene” is a contiguous region of genomic DNA that consists of the sequences that encode a polypeptide and substantially all of the sequences that regulate expression of the coding sequences.

A “regulatory pathway” is a collection of cellular constituents that regulate the expression of one or more gene products, wherein each cellular constituent is influenced according to some biological mechanism (e.g., cooperative binding, DNA or protein modification, etc.) by one or more other constituents of the collection.

An “array” is a plurality of different nucleic acids immobilized at positionally-addressable locations on a solid phase surface.

A “microarray” is an array in which the immobilized nucleic acids are located within a region of less than 6.25 cm² in size (although the solid phase surface can be much larger).

A “regulatory array” is an array of nucleic acids, each comprising a functional site sequence or functional site variant sequence.

A “pharmaceutical regulatory array” is an array of nucleic acids, each comprising a functional site sequence or functional site variant sequence associated with one or more specific genes known or presumed to be involved in pharmaceutical response or metabolism.

Arrays

In one embodiment, the invention provides arrays of polynucleotides comprising functional sites. Methods of preparing polynucleotides comprising functional sites and methods of preparing arrays comprising the same are described in detail below.

1. Functional Sites

In one embodiment, the invention provides arrays or microarrays comprising polynucleotides comprising, consisting essentially, or consisting of one or more functional sites, fragments, variants or complements thereof. The invention encompasses any and all functional sites of any and all genomes. For example, functional sites of the present invention include those identified or present in the genome of any animal, virus, or plant. In certain embodiments, functional sites include those present in a mammalian genome, such as, for example, a human, mouse, or pig genome. Functional site sequences may be identified by methods described herein.

The number and location of functional sites differs between and among cell types, as may the number and identity of the proteins that bind to the genomic locale to create a given functional site. Certain functional sites may be specific to a particular tissue cell type or to a restricted set of tissue or cell types (“tissue-specific functional sites”). Another set may form in co-ordination with the cell cycle or due to environmental or other stimuli, including drug treatment, for example. Other functional sites or variant functional sites may be associated with a disease or disorder. In addition, certain functional sites may be present in all tissue or cell types (“constitutive functional sites”) (e.g., Mol Cell Biol May 1999;19(5):3714-26).

The total number of potential functional sites within a given cell depends largely on the cell type and state, but is generally equal to at least the number of active genes within that cell, and may be many times that number as active genes may be surrounded by or contain, e.g., their introns or other non-coding regions, more than one functional site. Functional sites may function alone or in combination with other functional sites to modulate the expression of a cis-linked gene (e.g., Mol Cell Biol November 1999;19(11):7600-9), or even a receptive gene in trans. Indeed, it is understood that gene regulation is generally governed by the coordinate activities of multiple regulatory elements that may be present within one or more functional sites associated with a gene locus, which includes the coding region and regulatory regions.

The superset of functional sites is expected to contain active units from virtually all known classes of genetic regulatory elements including promoters, enhancers, silencers, locus control regions, domain boundary elements, and other elements having chromatin remodeling activities. Each of the aforementioned units may in turn be comprised of one or more functional site (e.g., Trends Genet October 1999;15(10):403-8). In addition, other processes may be controlled by a subset of the functional sites or interactions between them. These include, but may not be limited to, DNA replication, recombination and the structure of the genomic DNA within the nucleus such as regions of specialized chromatin structure and three-dimensional topology of the chromatin fibre. As such, the complete set of functional sites across all cells and tissue types will contain substantially all of the regulatory elements necessary to define the transcriptional program of the genome, in any state of differentiation or in response to any stimulus.

Functional sites represent a unique class of nucleic acid sequences and possess a variety of common physical and functional characteristics and attributes, as outlined below.

i. Size

Functional site sequences are generally size-restricted and biologically bounded by (1) the positions of flanking nucleosomes and (2) limits on the area of DNA over which thermodynamically stable nucleoprotein complexes may form. The extent of the functional site typically spans the inter-nucleosomal interval of approximately 150-250 bp. This interval corresponds to the size of sequence that is needed to place a nucleosome, and it has been a common assumption that functional sites represent a break in the cannonical nucleosomal array that constitutes the vast majority of chromatin.

In certain embodiments, a core domain within a functional site sequence can be identified which is restricted to a region of approximately 80-100 base pairs in length, over which DNA-protein interactions take place. It has been shown that the cooperative binding of transcription factors to such core regions are sufficient to exclude a nucleosome in vitro (Adams and Workman, Mol. Cell Biol., 15: 1405), and this has been accepted as a common mechanism for how these sites may form in vivo. Nucleosomal mapping experiments have shown that functional sites such as the Drosophila hsp26 promoter (Lu et al., EMBO J. 14; 4738) and the human β-globin HS2 (Kim and Murray, Int. J. Biochem. Cell Biol 33: 1183) are non-nucleosomal. It is thought that most functional sites are non-nucleosomal in nature (Boyes and Felsenfeld, EMBO J. 15: 2496; Wallrath et al., Bioessays 16:165). These conclusions are well-supported in the literature (e.g., ibid and Struhl K. Science. Aug. 10, 2001;293(5532):1054-5). However several functional sites are known to still have bound histone proteins and transcription factors, suggesting that the functional sites may exist in conjunction with a modified nucleosome.

Flanking sequences surrounding the core region appear to modulate the activity of this core region, though this effect tapers off sharply as the distance from the core region increases. The boundaries of the sequences needed for functional activity, e.g., hypersensitivity activity, can be defined functionally by performing deletional analysis in studies following stable transfection of cells (Philipsen et al., EMBO J. 9: 2159) or transgenic studies (Zhou et al., J Cell Sci. 108:3677). These approaches define the minimum extent of sequence required to retain the biological function associated with the functional site under examination.

ii. Clusters of Transcription Factors Binding Elements

High resolution studies of DNA sequences of known regulatory regions demonstrates that these regions often represent clusters of recognition sites for promoter-specific DNA-binding proteins (Emerson et al., 1985). Very few of these binding elements can be predicted on the basis of DNA sequence alone. Recent studies using chromatin immunoprecipitation have revealed that the ‘consensus’ binding motifs of transcription factors have both low sensitivity and very low specificity in predicting actual sites of in vivo DNA-protein interaction. However, this prediction can be substantially improved (and in many cases rendered definitive) with prior knowledge that the motif occurs in a region known to comprise a functional site.

iii. Catalytic Activity

Functional site-forming genomic DNA sequences have unique physical properties. In principle, these sequences can be said to function in a ‘catalytic’ manner that is analogous to the interaction between an enzyme and its substrate. These DNA sequences contribute to the free energy of formation of a nucleoprotein complex in a manner that dramatically increases its probability of activation vs. neighboring DNA regions.

An important finding has been that these sequences only function so when they are assembled into genomic chromatin. The sequences adopt a particular topological confirmation, which is compatible with the coalescence of numerous proteins, some in contact with DNA and some in contact with other proteins. This results in the formation of a nucleoprotein complex. The formation of the complex is precisely correlated with a particular sequence, which drastically lowers its activation energy with respect to other sequences, and also with respect to contact of those proteins with one another in vivo under random circumstances. The final product is stochastic, in the sense that it forms in an all-or-none fashion (e.g., Felsenfeld et al Proc Natl Acad Sci USA. Sep. 3, 1996;93(18):9384; Boyes & Felsnfeld EMBO J. May 15, 1996;15(10):2496).

The rate of formation can be measured through interrogation with the quantitative nucleosensitivity assay described below and in more detail in PCT Publication No. WO 02/097135 and U.S. patent application Ser. No. 10/157,027 and Ser. No. 10/319,440, which are hereby incorporated be reference in their entirety. When examined over a time-course of digestion, a characteristic ‘signature’ relationship can be derived for each catalytic sequence, which can be quantified and assigned a mathematical constant. A further conceptual parallel with other catalytic processes is that nucleoprotein complex formation can be manipulated through the introduction of point mutations or small deletions or insertions in the “active site” (critical DNA binding bases) or “allosteric” sites Ouxtaposed sequences). This principle has been demonstrated in numerous publications (e.g., Stamatoyannopoulos et al., EMBO J. Jan. 3, 1995;14(1):106).

iv. Intrinsic Ability to Form

A further defining feature of functional sites is that the function of the DNA sequence component—i.e. its complex-forming activity is intrinsic. The principal evidence for this is the fact that these sequences can be excised and inserted into other positions in the genome, where they exhibit the same functional chromatin activities. Substantial experimental experience from model systems has revealed that functional sites can form when included in either constructs used to create stably transfected cell lines (Fraser et al., 1990) or transgenic animals (Lowrey et al. Proc Natl Acad Sci USA. Feb. 1, 1992;89(3):1143-7; Levy-Wilson et al., 2000).

v. Activity in Transgenic Systems

Many functional sites can be shown to have regulatory influences on the expression of reporter genes when included in constructs in transfection or transgenic systems. Such systems can be used to demonstrate activities associated with promoters (Furbass et a., 2001), transcriptional enhancers (Levy-Wilson et al., 2000) and transcriptional silencers (Oritz et a., 1999). Functional sites have also been reported to behave as insulator elements, defined as sequences that prevent the transmission of chromatin structure features associated with the genomic location into which the construct has integrated, in various transgenic models (Li et al, 2002; Mustkov et al., 2002; Rivella et al., 2000). Functional sites can act as elements capable of opening chromatin, which may act singly (Nemeth et al., 2001) or in a coordinated fashion with other functional sites (commonly termed a Locus Control Region (Li et al., 2002; Shewchuk et al., 2001)).

As such, these transgenic assays represent a tool for identifying and classifying functional sites on the basis of function and also defining the minimum size of fragment on which the function is confined.

vi. Activity in Chromatin Reconstitution Systems

Functional sites can be included in templates for reconstitution protocols (Leach et al., 2002) or in vitro assembly systems (Becker et al., 1991) and are capable of directing the formation of chromatin structure similar to that detected in vivo.

vii. Nucleoprotein Complexes

In general, the majority of functional sites are believed to bind multiple (e.g., three or more—with an expected average of 6-7) DNA binding proteins, which may be, e.g., either ubiquitous transcription factors or proteins with a specific pattern of expression. The cooperative binding of transcription factors has been shown to be sufficient to exclude a nucleosome in vitro (Adams and Workman, 1995), and this has been accepted as a common mechanism for how these sites may form in vivo. Nucleosomal mapping experiments have shown that functional sites such as the Drosophila hsp26 promoter (Lu et al., 1995) and the human β-globin HS2 (Kim and Murray, 2001) are non-nucleosomal. It is thought that most functional sites are non-nucleosomal in nature (Boyes and Felsenfeld, 1996; Wallrath et al., 1994).

It has also been proposed and demonstrated that, in certain rare circumstances, some DNA sequences can form functional sites in the absence of protein binding (i.e., purely on the basis of their internal structural properties). Examples of these include the CpG-island associated with the human glucose-6-phosphate dehydrogenase gene that forms in yeast (Mucha et al., 2000) and sequences associated with repeats giving rise to human chromatin fragile sites (Hsu and Wang, 2002). Other functional sites have been identified in ternary complexes between the bound transcription factors, underlying DNA sequence and the still associated histones (Steger and Workman, 1997).

viii. Fractionation Properties

Typically, functional sites are embedded in accessible chromatin. Some of the discovered properties of accessible transcriptionally competent chromatin include increased generalized sensitivity to nuclease digestion, patterns of histone modification (accessible chromatin has high levels of histone acetylation) and higher solubility in moderate salt solutions (such as 150 mM NaCl and 3 mM MgCl₂). These properties allow the preparation of chromatin fractions enriched in functional sites (Spencer and Davie, 2001).

ix. Biological Activities

Focal alterations in chromatin structure, such as those associated with functional sites, are the hallmark of active regulatory sequences in eukaryotic genomes. These alterations display remarkably similar physical properties irrespective of genomic location or even of species of origin. Exemplary activities are provided in Table 1.

TABLE 1
Activities Associated with Functional Sites

| Property | Definition | Example | Reference |
|---|---|---|---|
| Promoter | Transcriptional | Murine retroviral | Bresnick et al., 1992 |
| | promoter | MMTV-LTR | |
| Transcriptional | Upregulates | Human β-globin | Kong et al., 1997 |
| Enhancer | transcription from | HS2 | |
| | linked gene | | |
| Transcriptional | Downregulates | Mouse Ig silencer | Liu et al., 2002 |
| Silencer | transcription from | | |
| | linked gene | | |
| Matrix Attachment | Tether chromatin to | MARs within human | Kieffer et al., 2002 |
| Region | protein backbone | CD8 gene complex | |
| Origin Replication | Origin of DNA | Puff II/9A ORI | Urnov et al., 2002 |
| (ORI) | replication | | |
| Recombination Sites | Sites of frequent | AML1/RUNX1 | Zhang et al., 2002 |
| | chromosome | breakpoints in t(8;21) | |
| | translocations | leukemia | |
| Structural Elements | | Human telomeres | Tommerup et al., |
| | | | 1994 |
| Unknown | Sequences capable of | Human HPFH- 1 | Elder et al., 1990 |
| | forming HSs may | enhancer | |
| | occur throughout | | |
| | genome | | |

x. Position Relative to Genes

An important feature of functional sites which has emerged (and, in some cases such as the globin genes, has been exhaustively investigated) is that the genomic proximity of a gene to a functional site is the principal determinant of the influence of that functional site on the regulation of that gene. Functional site sequences may be located upstream (5′), downstream (3′) or within genomic regions containing transcribed regions of a gene. Accordingly, functional sites may be located within transcribed regions of a gene.

xi. Repetitive Content

Functional site sequences can essentially be thought of as being unique in the genome, save in cases where the sequences lie in segmental duplications.

xii. Method of Identifying

Functional sites may also be defined or characterized based upon their method of identification, including, for example, the specific chromatin modifying agent (or combination thereof) used to isolate and identify the functional sites. Detailed methods of identification are described below, and in certain embodiments, functional sites of the invention include those sequences identified according to any one of these methods. In certain embodiments, functional sites are genomic sequences that are accessible to or modified by any DNA modifying agent, including those described infra.

2. Subsets and Combinations of Functional Sites

In certain embodiments, the invention includes arrays comprising a set or group of functional sites. These sets may be characterized by any means available, including, for example, the specific DNA cleaving or tagging agent used to identify the functional sites, the specific cell or tissue source of genomic DNA from which the functional sites were isolated (e.g. different drug treatment different tissue type or different treatment), or the genomic location of the functional sites, for example.

In certain embodiments, methods and compositions of the invention identifies (i.e. profiles) and includes functional sites identified from a specific tissue or cell. Further, these functional sites may be limited to those identified at a specific or identifiable biological point or condition, such as, for example a certain developmental stage, cell cycle state or diseased state. Accordingly, the present invention includes arrays comprising functional sites, or fragments or portions thereof, identified in the genome of specific cells or tissues. Similarly, the invention provides methods of profiling functional sites within specific cells or tissues. By identifying functional sites present in a particular cell type and/or at a specific biological condition, the invention provides a discrete genomic fingerprint, referred to as a “tissue regulotype” associated with the specific cell or tissue, which may be used to identify cells and identify genes that govern a variety of cellular processes, including, for example, cellular differentiation, specialized cell function, and/or disease establishment and/or progression.

A library or array of functional site sequences or sequence locations generated according to the invention provides rich and highly valuable information concerning the gene regulatory state of the cells from which the chromatin had been isolated. Further, two or more arrays or profiles (information obtained from use of an array) of such sequences are useful tools for comparing a sample set of functional sites with a reference, such as another sample, synthesized set, or stored calibrator. In using an array, individual nucleic acid members typically are immobilized at separate locations and allowed to react for binding reactions. Such positional addressability allows high throughput and reproducible analysis and comparison of functional sites from different samples. Primers associated with assembled sets of functional sites are useful for either preparing libraries or arrays of sequences or directly detecting functional sites from cell samples.

In many embodiments made possible from this discovery, genomic regulatory information is extracted from a biological sample without foreknowledge of genetic locus or marker information. That is, exemplified methods can identify en mass, functional sites for which no genetic marker has been identified previously. After identification, DNA containing sequences of the functional sites may be used as probes to identify complementary genomic DNA sequences to find proteins and protein complexes having regulatory activity, and to discover pharmaceutical drug activities for compounds that can influence one or multiple regulatory systems. In addition, knowledge of these sequences allow the mapping and detection of naturally occurring mutations in the genome which are implicated in causing, potentially pathogenic, changes to the transcriptional program of the cell, such as single nucleotide polymorphisms (SNPs). In many embodiments, the sequences are grouped into libraries, which can be converted or abstracted into arrays to probe multiple regulatory systems simultaneously.

A library (or array, when referring to physically separated nucleic acids corresponding to at least some sequences in a library) of functional sites has very desirable properties as further detailed below. These properties can be associated with specific cell types and cell conditions, and may be characterized as regulatory profiles. A profile, as termed here refers to a set of members that provides regulatory information of the cell from which the functional sites are obtained. A profile in many instances comprises a series of spots on an array made from deposited functional site sequences. Without wishing to be bound by any one theory of this embodiment of the invention, it is believed that a eukaryotic cell such as a human cell contains many potential functional sites and that only a portion of the functional site potential regulatory elements are formed at any given time. By sampling and profiling the functional sites, an array presents a snapshot of the cell's regulatory status.

An array of the invention typically comprises at least 10, more preferably at least 100, 250, 500, 1000, 2000, 5,000 and even more than 10,000 polynucleotides comprising functional sites. An array profile of a cell's regulatory status typically concerns at least 10, more preferably at least 100, 250, 500, 1000, 2000, 5,000 and even more than 10,000 ACEs in some cases. Profile information from a test sample may be more or less detailed depending on the number of functional sites required to distinguish the profile from others. For example, a profile designed to examine the presence of a particular chromosomal breakage crosslinkage or other defect may need to detect only 2-3, 2-10, 3-5, 10-20 or other small number of functional sites. With present techniques, the activation state (defined by an ability to form a functional site in chromatin) of only one or a very limited number of such sequence elements may be detected in an single experiment, such as a southern blot analysis. The arrays of the invention allow the simultaneous analysis of many more functional sites.

In one embodiment of the invention, array profiles may be generated using arrays comprising random functional sites or functional sites of unknown sequence. In preferred embodiments, arrays comprising specific functional sites may be utilized, including, for example, functional sites identified as being associated with one or more genetic loci. While the sequence of functional site used in arrays is desirous, it is not necessary.

A characteristic profile generally is prepared by use of an array. An array profile may be compared with one or more other array profiles or other reference profiles. The comparative results can provide rich information pertaining to disease states, developmental state, susceptibility to drug therapy, homeostasis, and other information about the sampled cell population. This information can reveal cell type information, morphology, nutrition, cell age, genetic defects, propensity to particular malignancies and other information. Accordingly, particularly desirable embodiments were explored that use arrays for creating functional site libraries, as detailed below.

The simultaneous detection of multiple functional sites using arrays provides a wide range of methods for a variety of advantages. In some embodiments, an array contains one or more internal references and the data profile is used directly without further comparison with reference data. In other embodiments, a library of sites (either sequences, position locations or both) is obtained from a sample and then compared with another library, such as a pre-existing “type” library. A type library may be characteristic for a cell type, a development status type, a disease type such as a genetic disease, or a morphologic type associated with the presence of factor(s) such as hormones, nutrients, pharmacologically active compounds and the like. The comparison to a type library may generate an output set of difference “profile information” for the library.

The term “library” as used here means a set of at least 10, preferably 50, 100, 200, 300, 500, 1000, 2000, 5000, 10,000, 20,000 30,0000 or even at least 50,000 members of nucleic acids having characteristic sequences. The library may be an information library that contains a) functional site sequences, b) location information for functional sites in the genome; or c) both sequence information and matching location information. As an information library, the members preferably are stored in a computer storage medium as sequences and/or gene position locations. As a physical DNA library, the members may exist as a set of nucleic acids, clones, phages, cells or other physical manifestations of DNA in a form useful for simultaneous manipulation.

A library of nucleic acid molecules conveniently may be maintained as separate cloned vectors in host cells. Preferably each member is physically isolated from the other members, although a mixture of members within a common vessel may be suitable, particularly for assays wherein members become separated based on a physical property such as by hybridization with specific members on a solid support.

A functional site library member in most instances comprises a sequence at least 16 bases long and less than 1500 bases long. More preferably the sequence comprises between 60 bases and 400 bases. Yet more preferably the sequence comprises between 75 bases and 300 bases. The term “mean sequence length of the functional site sequences” means the numeric average of all DNA sequences in the respective library or array. Experimental results indicate that most functional sites are about 50 to 400 bases long and more generally about 150 to 300 bases long. However, the skilled artisan would appreciate that the length of functional sites may be quite variable, as a functional site may include one or more regulatory sequences, may be associated with different polypeptides or complexes, and/or may contain various degrees of chromatin modification. Methods for replicating DNA (or RNA) sequences and maintaining copies of those sequences in libraries are well known and have been used for some years. See for example the procedures described in U.S. Nos. 4,987,073; 5,763,239; 5,427,908; 5,853,991. In certain embodiments, the invention includes only newly identified functional sites or sequences.

The invention further includes combinations and groupings of functional sites. Each individual functional site is involved in the regulation of one or more genes. However, combinations of functional sites typically coordinately regulate genes. That is, it was found that many functional sites can work together, as will be appreciated by a skilled artisan. Many of these combinations are seen as clusters physically located on the same chromosome or near a certain gene, for example. However, other functional sites coordinately control expression, even though they are found in disparate regions of the genome. These groups are identified by assays that detect their effects, such as arrays that compare whether the functional sites of the invention are active in particular cell types or under particular conditions such as growth conditions or chemical or environmental exposures. Functional sites that are present or active in the same or similar cells or conditions are likely involved in the coordinate regulation of one or more genes. Accordingly, in certain embodiments, the invention provides arrays of functional sites associated with a particular gene or cluster. Such functional sites may be associated with a specific chromosome, and may be within a specific distance from each other, including, for example, within 100 bp, 500 bp, 1 kb, 2 kb, 5 kb, 10 kb, 100 kb, or greater than 100 kb.

3. Complements, Variants and Fragments of Functional Sites

The invention also includes arrays comprising polynucleotides comprising variants and complements of polynucleotide sequences of the invention. Complements may be used for a variety of purposes, including, for example, to detect the presence of a functional site sequence. In certain embodiments, complements are completely complementary to a polynucleotide sequence of the invention, including fragments thereof. However, the skilled artisan would understand that it is not required that complements are completely complementary to the entirety of a polynucleotide of the invention. In certain embodiments, complements are complementary to a portion of any polynucleotide of the invention and may be less than completely complementary. In specific embodiments, however, complements of the invention are capable of hybridizing to a polynucleotide of the invention under stringent or moderately-stringent conditions, as set forth below. As such, complements include oligonucleotides, such as those suitable for performing polymerase chain reaction.

The invention includes variants of polynucleotides of the invention and complements thereof. Examples of specific variants include allelic variants, including those associated with a disease and homologs from different organisms or species. Typically, polynucleotide variants will contain one or more substitutions, additions, deletions and/or insertions. Variants also encompass homologous genes of xenogenic origin.

The invention includes variants lacking one or more functions associated with the corresponding functional site of the invention, e.g. the ability to bind a polypeptide bound by the functional site, the ability to regulate gene expression in the same manner as the functional site, or the ability to be identified according to the procedures described herein to identify functional sites. In certain embodiments, a variant is associated with a disease.

In other embodiments, variants retain one or more functions associated with the corresponding functional site. Functional sites of the invention typically form nucleoprotein complexes by binding one or more proteins. The skilled artisan would recognize that such binding may not require the exact sequence of a functional site of the invention and that certain nucleotide deletions, additions, or substitutions may be tolerated without substantially or completely preventing binding. Indeed, it has been shown that protein binding nucleic acid sequences frequently comprise a consensus sequence, which may consist of the core nucleotides required for protein binding. Accordingly, functional variants of the invention include polynucleotides with an altered sequence as compared to an identified functional site, but which retain one or more physical or functional properties of the functional site, including any of the propertied described above, the ability to affect transcription of a linked gene, or the ability to bind the same polypeptide as the native sequence, for example. Such binding may be determined by any method available in the art, including, for example, electrophoretic mobility shift assays performed in the presence or absence of an antibody specific for the polypeptide that binds the native polynucleotide.

Variants of the invention may be identified by a variety of means, including sequence homology to a polynucleotide of the invention or the ability to hybridize to a polynucleotide sequence of the invention or complement thereof. In certain embodiment, the invention includes polynucleotides with at least 60% identity, at least 70% identity, at least 80% identity, at least 90% identity, at least 95%, or any integer value between and including 70% and 99% identity, to a polynucleotide of the invention, including a functional site or fragment or complement thereof. In one embodiment, the invention includes variants that are single nucleotide polymorphisms of functional sites. The skilled artisan would recognize that hybridization conditions, including those described within supra, may be tailored to detect single nucleotide variations in sequence, and, accordingly, the methods of the invention may be used to identify single nucleotide polymorphisms in functional site sequences, including those that may be implicated in disease.

The term sequence homology, as described herein, refers to the sequence relationships between two or more nucleic acids, polynucleotides, proteins, or polypeptides, and is understood in the context of and in conjunction with the terms including: (i) reference sequence, (ii) comparison window, (iii) sequence identity, (iv) percentage of sequence identity, and (v) substantial identity or homologous.

(i) A reference sequence refers to a sequence used as a basis for sequence comparison. A reference sequence may refer to a subset of or the entirety of a specified sequence or complement thereof.

(ii) A comparison window includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer. Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol Biol. 48: 443 (1970); by the search for similarity method of Pearson and Lipman, Proc. Natl. Acad. Sci. 8: 2444 (1988); by computerized implementations of these algorithms, including, but not limited to: CLUSTAL in the PC/Gene program by lntelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Higgins and Sharp, CABIOS :11-13, 1989; Corpet, et al., Nucleic Acids Research, 16:881-90,1988; Huang, et al., Computer Applications in the Biosciences 8:1-7,1992; and Pearson, et al., Methods in Molecular Biology 24:7-331,1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs using default parameters. Altschul et al., Nucleic Acids Res, 2:3389-3402, 1997. It is to be understood that default settings of these parameters can be readily changed as needed in the future.

(iii) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions.

(iv) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(v) (i) The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and most preferably at least 95%. It further includes sequences with at least 70-99% sequence identify, including all integer values in-between, including, for example, 90, 91, 92, 93, 94, 95, 96, 97, and 98.

Another indication that nucleotide sequences are substantially identical is if two molecules hybridize to each other under stringent conditions. The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary, non-limiting stringent hybridization conditions are as following: 50% formamide, 5×SSC, and 1% SDS, incubating at 42° C., or, 5×SSC, 1 SDS, incubating at 65° C., with wash in 0.2×SSC, and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec.-2 min., an annealing phase lasting 30 sec.-2 min., and an extension phase of about 72° C. for 1-2 min.

Nucleic acids that do not hybridize to each other under stringent conditions can be still substantially identical if they hybridize under moderately stringent conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

In certain embodiments, the invention includes arrays of fragments of functional sites. Typically, arrays of the invention are useful in detecting hybridizing nucleic acids. Such specific hybridization does not necessarily require a complete functional site sequence, and it is understood that fragments of functional sites are sufficient to produce specific hybridization as required by methods of the invention. It is also understood, as described above, that functional sites typically contain a core region associated with functional activity, as well as flanking regions. Accordingly, the invention includes fragments and regions of functional sites, including fragments consisting of or comprising core regions of functional sites. In certain embodiments, such fragments possess at least one physical or functional characteristic of the functional site from which they were derived. Functional fragments may be identified based upon any associated biological, biochemical, or physical function and by any available means. Thus, functional fragments of the invention include fragments capable of affecting or regulating (e.g. increasing or reducing) transcription of an operatively-linked gene, capable of binding to a transcription factor, capable of recruiting a transcriptional cofactor, capable of being methylated, and capable of directing methylation, demethylation, acetylation, deacetylation, or any other modification of genomic DNA or chromatin, for example. Furthermore, it is not necessary that the functional fragment possesses the associated function in isolation; rather, a functional fragment may require the presence of additional regulatory or other nucleic acid sequences to function.

In one embodiment, a functional site fragment comprises between 10 and 75 bases of a functional site sequence. In another embodiment, a nucleic acid may comprise between 12 and 30, 15 to 50, 50 to 300, 100 to 200 or all of a functional site sequence. In most instances, at least 10 bases of a sequence desirably are used, preferably at least 20, and more preferably at least 50 bases. For example, fragments may comprise at least about 10, 15, 20, 30, 40, 50, 75, 100, 150, 200, 300, 400, 500 or 1000 or more contiguous nucleotides of one or more functional site sequences as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

In another embodiment, the invention includes fragments of functional site polynucleotides that do not possess a functional activity associated with the functional site. Such fragments may include, for example, probes or primers suitable for identifying, selecting or amplifying polynucleotides. Probes and primers of the invention include those corresponding to a region of a functional site or a complement thereof. In certain embodiments, probes and primers are preferably greater than 6 bases long, greater than 8, 10, 12, 16, or greater than 20 bases long. The term nucleic acid probe or oligonucleotide probe refers to a nucleic acid capable of binding to a target nucleic acid of complementary sequence through one or more types of chemical bonds, usually through complementary base pairing and usually through hydrogen bond formation. As used herein, a probe includes natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes may be directly labeled with isotopes, such as, for example, chromophores, lumiphores, or chromogens, or indirectly labeled, such as with biotin to which a streptavidin complex may later bind. The presence or absence of a target polynucletoide sequence of interest, such as a functional site, in a sample may be readily determined by determining the binding of a probe to the sample or the amplification of a PCR product from the sample.

In many embodiments, functional sites and other polynucleotides of the invention are used at least in one stage as an isolated nucleic acid. The term isolated means a material that is at least partially free from components that normally accompany the material in the material's native state. Isolation connotes a degree of separation from an original source or surroundings. Isolated, as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man. By way of example and not limitation, a nucleic acid or peptide that is 0.1% pure in a biological sample becomes “isolated” when it is purified to at least 0.2% purity. In certain embodiments, the isolated material will become substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. An isolated DNA molecule prepared by chemical synthesis or enzymatic synthesis from cDNA represents another common example of isolated DNA. A skilled artisan knows a wide variety of procedures for preparing such isolated DNA via removing contaminants, thus making the DNA more homogeneous.

Nucleic acids that contain functional sites may be of a variety of types, including deoxyribonucleotides or ribonucleotides and polymers thereof in either single- or double-stranded form. The term encompasses nucleic acids containing known nucleotide analogs or modified backbone residues or linkages, including synthetic, naturally occurring, and non-naturally occurring, which have similar binding properties as the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs).

Functional site sequences may be identified, manipulated, characterized and/or used according to illustrative methods provided herein below, and, in addition, according to the disclosures of U.S. Ser. No. 09/432,576, filed Nov. 12, 1999, entitled “Production of Nuclease Hypersensitive Site Libraries”; U.S. Serial No. 60/378,664, filed May 9, 2002, entitled “DNA Microarrays Comprising Regulatory Elements and Comprehensive Profiling Therewith”; U.S. Ser. No. 10/319,440, filed Dec. 12, 2002, entitled “DNA Microarrays Comprising Regulatory Elements and Comprehensive Profiling Therewith”; U.S. Ser. No. 10/187,887, filed Jul. 3, 2002, entitled “Global Isolation of Functionally Active Genomic Elements”, PCT/US02/16967, filed May 30, 2002, entitled “Accurate and Efficient Quantification of DNA Sensitivity By Real-Time PCR,” and U.S. Provisional Patent Application “Profiled Regulatory Sites Useful for Gene Control,” filed Dec. 5, 2002.

4. Identification of Functional Sites

A variety of methods may be employed to identify and isolate functional site sequences of the invention. Such methods may also be employed to isolate DNA fragments used for probing arrays of the invention. Detailed descriptions of methods of identifying and isolating functional sites are provided in U.S. Provisional Patent Applications No. 60/108,206, No. 60/302,369, and No. 60/290,036, U.S. patent application Ser. No. 09/432,576, Ser. No. 10/187,887, Ser. No. 10/157,027, and Ser. No. 10/319,440, PCT Publication No. WO 02/097135, and PCT Application No. PCT/US02/15032, which are hereby incorporated by reference in their entirety. In addition, polynucleotides may be cloned from genomic libraries by routine procedures, including, or example, polymerase chain reaction, or synthesized using techniques well known in the art.

In one embodiment, a general method of identifying functional sites includes the basic steps of: (1) treating nuclear chromatin with an agent that cleaves or tags DNA at functional sites; and (2) isolating DNA segments flanking cleavage sites or tagged sites. In addition, the isolated DNA segments may be subcloned into a vector. The basic method may also be performed using in vitro assembled chromatin constructs. In one embodiment, the method further includes the step of amplifying the isolated DNA segments before subcloning, preferably by PCR.

A variety of agents may be used to cleave or tag functional sites. Any agent capable of detecting a focal alteration in chromatin structure may be employed to identify functional site sequences. Functional sites are modified by the action of one or more of these factors on the biological sample, the best documented and recognized example of which is the action of the non-specific endonuclease DNAse (e.g. EMBO J 14:106-16 (1995)). Non-specific endonucleases, such as Dnasel, are typically used to discover functional sites, but other agents can be used just as well. Potentially a subset of functional sites will not be detected by DNAse I and sets of functional sites may alternatively be identified by the actions of nucleases (both sequence-specific and non-specific), endogenous and exogenous); topoisomerases; methylases; acetylases; chemicals; pharmaceuticals (e.g. chemotherapy agents); radiation; physical shearing; nutrient deprivation (e.g. folate deprivation); etc. Essentially any agent, whether biological (e.g. enzymes), chemical (e.g. DNA binding molecules), or physical (e.g. stress), which will modify DNA in the nucleus, which is not occluded in the folded chromatin structure but exists in open regions accessible to DNA binding activities and is, hence, more liable to break. For example, modifications of the DNA in the nucleus, such as the action of dam methylase, can be used as a marker when the DNA is subsequently purified, for example, by the use of restriction enzymes that are differentially sensitive to dam methylation. Exemplary classes of these agents and examples of such are set forth in Table 3.

TABLE 3
Agents Suitable for Detection of Functional Sites

| Class | Description | Example | Site examined | Reference |
|---|---|---|---|---|
| Non-specific | Endonucleases with | DNaseI, | Chicken | Wood and |
| nucleases | little or no cutting | DNaseII, | globin 5' | Felsenfeld, |
| | specificity | Micrococcal | HS1 | 1982 |
| | | nuclease | | |
| Endogenous | | DNaseI | | |
| nucleases | | | | |
| Restriction | Sequence-specific | Pvu II, Nhe I | Chicken | Boyes and |
| endonucleases | endonucleases | | erythroid- | Felsenfeld, |
| | | | specific | 1997 |
| | | | ^{A}/ - | |
| | | | globin | |
| | | | enhancer | |
| Modified DNA- | Synthetic proteins | Sp1 + | Human | Kuo et al., |
| binding proteins | capable of binding | nuclease tail | MnSOD | 2002 |
| | within sites of interest | (PIN*POINT) | promoter | |
| | and inducing cutting or | | | |
| | modification | | | |
| DNA modifying | DNA-binding enzymes | dam DNA | lacZ reporter | Wines et al., |
| enzymes | which modify their | methyl- | gene in | 1996 |
| | binding site | transferase | Drsophila | |
| | | | nuclei | |
| Intercalator | DNA minor and major | Bleomycin | | |
| agents | groove intercalators that | | | |
| | cause strand breakage | | | |
| Topoisomerases | Naturally-occurring | Topo II | | |
| | nuclear enzymes that | | | |
| | change DNA linking | | | |
| | number via single- or | | | |
| | double-strand breakage, | | | |
| | DNA strand rotation, | | | |
| | and re-ligation | | | |
| Viruses | Viruses that integrate | | | |
| | into the genome | | | |

Alternatively, specific classes of functional sites may be targeted. For example, those known to be bound by a specific protein can be enriched for either by adding exogenous modified protein, which binds to its recognition site with in the functional site and induces modification (e.g. by creating a chimeric DNA-binding protein with a methylase or by incorporation of cross-linking reagents such as 4-azidophenacylbromide (e.g. Proc. Natl. Acad. Sci USA 89: 10287-10291) or strand damage (e.g. by incorporation of 1251, the radioactive decay of which would cause strand breakage (e.g. Acta Oncol. 39: 681-785 (2000)). Advantag can also be taken of such proteins bound in their natural context by isolating the nucleoprotein complexes in chromatin containing such proteins via antibody recogniztion (the ChIp protocol, Orlando et al., Methods 11:205-214 (1997)).

An alternate approach is to produce functional site enriched samples by fractionation. Digestion of nuclei will create a population of fragments where the smaller ones are more likely to have one or more cut sites within functional sites. That is as, dependent on the digestion conditions, either a functional site has received more than one cut to produce a small fragment whereas the background remains large. Alternatively, the functional site has been cut once, but the average distance between a functional site-cut and random cut or shear site is smaller than the average size of the entire population. Fragments can be separated on the basis of their size, before or after purification of the DNA from chromatin, by various methods including ultracentrifugation, preparative gel electrophoresis or size exclusion columns. If the fragments are isolated from the nuclei as chromatin fractions, they can be further enriched for functional site-containing material prior to centrifugation on the basis of properties of the nucleoprotein complexes that distinguish them from bulk chromatin. These include, for example, higher salt solubility of active chromatin domains (Ridsdale et al. Nucl. Acids. Res. 16:5915-5926 (1988)), the reactivity of thiol groups on the histone H3 (Chen-Cleland et al., J. Biol. Chem. 268:23409-23416 (1993)) and the extraction of nucleosomal DNA by binding to sulfated polysaccharides, such as heparin (Watson et al., J. Biol. Chem. 274:21707-21703).

Similarly, a variety of different methods may be utilized to isolate DNA segments containing functional sites, including the use of linkers, streptavidin/biotin, magnetic beads, and ab/hapten systems, for example. In certain embodiments, isolated functional sites may be labeled, e.g. when used to probe an array. The labeling of functional sites is achieved by standard methods, e.g., performing amplifications (linear or exponential) using synthetically labeled oligonucleotides (e.g. containing Cy5- or Cy3-modified nucleotides or amino allyl modified nucleotides, which allow for chemical coupling of dye molecules post-amplification), or by direct incorporation of modified nucleotides during the reaction.

Additional embodiments of methods of identifying functional sites include using subtractive methods designed to enrich functional site sequences and/or identify cell-specific functional sites. Subtractive methods may also be employed to remove repetitive sequences.

Another embodiment of the method of identifying functional sites involves concatamerizing isolated DNA segments, typically after further digesting the isolated fragments with a type IIs restriction enzyme to generate fragments of uniform size. The concatamer approach permits the sequencing and identification of multiple functional sites within a single polynucleotide sequence. In certain embodiment, linker sequences may be attached to one or more ends of the isolated fragments prior to concatamerization, typically by ligation. The boundaries of each isolated DNA segment, comprising a functional site, is readily determined by identifying the restriction site sequence or linker sequence located at one or both ends of each isolated DNA segment within the polynucleotide produced upon concatamerization.

In one embodiment, the sensitivity of a region of genomic DNA to DNA-modifying agents is quantified using Real-Time PCR. Such methods allow quantitative characterization of the activity of functional sites and the identification of functional sites with cell-specific or disrupted activities. The method generally involves isolating chromatin, treating a portion of the chromatin with a DNA modifying agent, treating another portion of the chromatin with the DNA modifying agent under modified conditions, isolating treated DNA from each portion, amplifying the candidate region by Real-Time PCR from each portion, determining copy number of the candidate region, and comparing to a reference curve to obtain relative copy number ratio of the candidate region and the reference region. Thus, the sensitivity of the candidate region to the DNA modifying agent is thereby determined relative to the sensitivity of the reference region. Embodiments of this method may also be used to detect single stranded nicks and to quantify naturally occurring single stranded DNA structures in vivo.

Typically, the identification and isolation of functional sites involves the treatment of genomic or chromosomal DNA with an agent that modifies DNA is some manner, such as cleaving one or both strands of DNA. However, there is no requirement that the genomic DNA is isolated or purified prior to treatment. Rather, treatment may be performed on whole cells, and preferably, treatment is performed on isolated nuclei. Thus, the treatment of genomic DNA is preferably performed in the context of chromatin inside a nucleus.

Another embodiment for the identification and isolation of functional sitesinvolves modifying the proteins that bind to a given functional site (or set of functional sites) so they induce DNA modification such as strand breakage. Proteins can either be modified by many means, such as incorporation of 1251, the radioactive decay of which would cause strand breakage (e.g., Acta Oncol. 39: 681-685 (2000)), or modifying cross-linking reagents such as 4-azidophenacylbromide (e.g., Proc. Natl. Acad. Sci. USA 89: 10287-10291) which form a cross-link with DNA on exposure to UV-light. Such protein-DNA cross-links can subsequently be converted to a double-stranded DNA break by treatment with piperidine.

Yet another embodiment for the identification and isolation of functional sites relies on antibodies raised against specific proteins bound at one or more functional sites such as transcription factors or architectural chromatin proteins, and used to isolate the DNA from the nucleoprotein complexes associated with functional sites in vivo. An example of a currently used technique cross-links proteins and DNA within the eukaryotic genome following treatment with formaldehyde. After isolation of the chromatin and following either sonication or digestion with nucleases the sequences of interest are immunoprecipitated (Orlando et al. Methods 11: 205-214 (1997)). In one illustrative assay according to this embodiment, the Chromatin Immunoprecipitation (ChIp) assay is used for the recovery of DNA sequences from eukaryotic nuclei by antibody recognition of epitopes present on associated proteins within the nucleoprotein complex. This approach can thus be used to recover DNA on the basis of either the enzymatic modifications of the histone proteins (referred to as the histone code and including but not limited to histone H4 and H3 acetylation, histone H3 methylation, histone H1 phosphorylation) or the presence of specific proteins (be they members of the basal transcriptional machinery or certain transcription factors) or post-translationally modified versions of such proteins (which can be modified in a similar way to histone proteins). Once the antibody recognition has been used to isolate the nucleoprotein complex the recovered DNA can be used to make one or more probes as described herein; e.g., pull-down probes, direct monotag probes or, following restriction, indirect monotag probes.

The CHIp protocol described above may be performed using any reagent capable of binding any protein associated with a regulatory sequence or functional site, either directly or indirectly. Accordingly, binding reagents, such as antibodies, may be directed to chromatin-associated proteins, such as histones, for example, protein components of the basal transcription machinery, proteins associated with DNA replication, DNA binding proteins, such as transcription factors, and proteins present in transcriptional complexes, such as coactivators and corepressors. Specific targeted histones may include, for example, histones H1, H2A, H2B, H3, and H4. Protein components of the basal transcription machinery that may be targeted include, for example, RNA polymerases, including poII, poIII and poIIII, TBP and any other component of TFIID, including, for example, the TAFs (e.g. TAF250, TAF150, TAF135, TAF95, TAF80, TAF55, TAF31, TAF28, and TAF20), or any other component of the poIII holoenzyme. In certain embodiments of the invention, functional sites associated with specific transcription factors, coactivators, corepressors or complexes may be isolated. Such transcription factors may include activators or repressors, and they may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes. For example, transcription factors such as c-myc, Rel/Nf-kB, neuroD, c-fos, c-jun, and E2F may be targeted. Antibodies directed to any transcriptional coactivator or corepressor may also be used according to the invention. Examples of specific coactivators include CBP, CTIIA, and SRA, while specific examples of corepressors include the mSin3 proteins, MITR, and LEUNIG. Furthermore, other proteins associated with transcriptional complexes, such as the histone acetylases (HATs) and histone deacetylases (HDACs) may be targeted.

Certain illustrative strategies that may be employed in accordance with this embodiment include the following. In one example, a ChIp pull-down probe can be used to query a standard array spanning some genomic sequences, for example contiguous 250 bp fragments spanning 50-100 kb of a gene locus, in order to determine the patterns of epigenetic modifications and correlate them with previously determined expression and structural data. In another example, a reiteration of the above experiment identifying functional site DNA by ChIp analysis can be performed with one or more members of a comprehensive collection of antibodies having specificity for histone modifications in order to generate a detailed description of the ‘histone code’ across a locus. In another example, by preparation of the ChIp-material from a range of transcriptionally permissive and non-permissive cells and tissues, or following the effects of the histone code following environmental stimuli or induction of a gene with specific chemicals, one can deduce the in vivo sequence of events which control or contribute to transcriptional regulation. In another example, the method involves assaying the effect of a class of potentially therapeutic molecules which are designed to modify the activities of the histone modifying enzymes not only on a gene of interest (as with locus profiling) but also by scanning large sections of the genome by creating in parallel an indirect monotag probe and hybridizing to appropriate tiling arrays.

In a related embodiment, multimodality profiling, e.g., combination probing with DNA modification agents, such as DNAse I, for example, and ChIP reagents, is performed using the arrays of the present invention. For example, as an alternative to performing sequential screens with DNA reagents prepared by one of the discussed selection techniques (such as sensitivity to nucleases or chemicals, selection of nucleoprotein complexes by antibodies etc.) is to perform the selections in parallel, for example performing a ChIp protocol with an antibody raised against histone H4 acetylation and then reselecting that population with a second antibody raised against a different modification. Similar combinations of ChIp with nuclease/chemical sensitivity selections can be analyzed, as can the methylation status of any preselected population. Functional site sequences identified and isolated from these populations can then be used in accordance with the arrays and methods described herein.

In another embodiment, alterations to the epigenetic pattern are also known to correlate with alterations with the activity of functional sites. One of the most closely studied types of modification is cytosine methylation. The global pattern of methylation is relatively stable but certain genes become methylated if they are silenced or conversely demethylated if activated. Differential methylation can be detected by use of pairs of restriction endonucleases that cut the same site differently according to whether or not it is methylated (Tompa et al. Curr. Biol. 12: 65-68 (2002)). Alternatively, it is possible to generically distinguish between a methylated and non-methylated cytosine by genomic sequencing (a methodology developed by Pfeifer et al. Science 246: 810-813 (1989)) that converts cytosine to uracil, which behaves similarly to thymine in sequencing reactions, and leaves methyl-cytosine unmodified. This material can be used as a template in PCR with primers sensitive to the C to U transition. Alternatively the potential mismatch (G:U) between oligonucleotide and template can be cleaved by E. coli Mismatch Uracil DNA Glycosylase, and that fragment removed from the population.

Additionally, in another embodiment, the enzymatic machinery which gives rise to or maintains the epigenetic patterns can also be labeled as described above so that it can be induced to cause detectable DNA modifications such as double stranded DNA breaks. Target proteins for this kind of approach would include the recently described HATs (Histone-Acetyl Transferases), HDACs (Distone De-Acetylase Complexes) whose effect on transcriptional induction has been recently described (Cell 108: 475-487 (2002)), as well as DNA methyltransferases and structural proteins that bind to the sites of methylation, such as MeCP1 and MeCP2. Histones and transcription factors are also known to become methylated, phosphorylated and ubiquinated. A range of covalent modifications, some of which have yet to be described, may be made to the structural and enzymatic machinery of transcription, replication and recombination. Current understanding indicates that such modifications have a regulatory role and it has been demonstrated that these modifications can be positively and negatively correlated with the functional activity of the underlying sequence (Science 293: 1150-1155). The potential for combinations of modifications of the functional sites overlays another layer of complexity of regulation on the underlying genome, and it is possible to dynamically follow these epigenetic changes with the immunoprecipitation of the DNA sequences from in vivo nucleoprotein complexes.

Functional sites define certain features of the nuclear architecture which play a large role in regulation of genomic processes. Increasingly, the molecules, including proteins and RNAs, which control the structure of the nucleus are being identified, and these are also used as targets to identify functional sites.

Moreover, cytologically distinct region of interphase nuclei have been described such as the nucleoli which contain the heavily transcribed rRNA genes (Proc. Natl. Acad. Sci. USA 69: 3394-3398 (1972)) and active genes may be preferentially associated with clusters of interchromatin granules (J. Cell Biol. 131: 1635-1647 (1995)). Specific regulatory regions may become localized to distinct areas within the nucleus on transcriptional induction (Proc. Natl. Acad. Sci. USA 98: 12120-12125 (2001)). By contrast, specific areas of eukaryotic nuclei have been shown to be transcriptionally inert (Nature 381: 529-531 (1996)) and associated with heterochromatin. Fractionation of the nucleus on the basis of such and similar physical properties can be used to capture sets of functional sites implicated in these processes.

5. Methods of Manufacturing Arrays

Microarrays are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences.

Microarrays are particularly desirable for their virtues of high sample throughput and low cost for generating profiles and other data. A DNA microarray typically is constructed with spots that comprise polynucleotide sequences comprising functional sites, or fragments, complements, or variants thereof. In a preferred embodiment, immobilized DNAs have sequences that hybridize to functional sites such as putative genomic regulatory elements. Arrays of the invention preferably contain polynucleotide at positionally addressable locations on the array surface.

Microarrays according to embodiments of the invention may include immobilized biomolecules such as oligonucleotides, cDNA, DNA binding proteins, RNA and/or antibodies on their surfaces. Any biomolecule capable of preferentially binding one or more functional sites may be used according to the invention to screen a sample for the presence of functional site sequences. Advantageous embodiments of the invention have immobilized polynucleotides (i.e. nucleic acid) on their surfaces. The nucleic acid participates in hybridization binding to nucleic acid prepared from functional sites which are differentially sensitive or hypersensitive to CMAs.

Polynucleotides comprising functional sites, variants, fragments or complements thereof, may be applied to an array in a number of ways. For example, the DNA sequence may be amplified using the polymerase chain reaction from a library containing such sequences, and subsequently deposited using a microarraying apparatus. In another way, the DNA sequence is synthesized ex situ using an oligonucleotide synthesis device, and subsequently deposited using a microarraying apparatus. In yet another way the DNA sequence may be synthesized in situ on the microarray using a method such as piezoelectric deposition of nucleotides. The number of sequences deposited on the array generally may vary upwards from a minimum of at least 10,100, 1000, or 10,000 to between 10,000 and several million depending on the technology employed.

Arrays of the invention may be prepared by any method available in the art. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized cDNA probes for directed deposition onto chip surfaces (see, e.g., U.S. Pat. No. 5,874,554).

Other useful technology that may be employed is the contact-print method developed by Stanford University, which uses high-speed, high-precision robot-arms to move and control a liquid-dispensing head for directed cDNA deposition and printing onto chip surfaces (see, Schena, M. et al. Science 270:467-70 (1995)). The University of Washington at Seattle has developed a single-nucleotide probe synthesis method using four piezoelectric deposition heads, which are loaded separately with four types of nucleotide molecules to achieve required deposition of nucleotides and simultaneous synthesis on chip surfaces (see, Blanchard, A. P. et al. Biosensors & Bioelectronics 11:687-90 (1996)). Hyseq, Inc. has developed passive membrane devices for sequencing genomes (see, U.S. Pat. No. 5,202,231). These methods and adaptations of them as well as others known by skilled artisans may be used for embodiments of the invention.

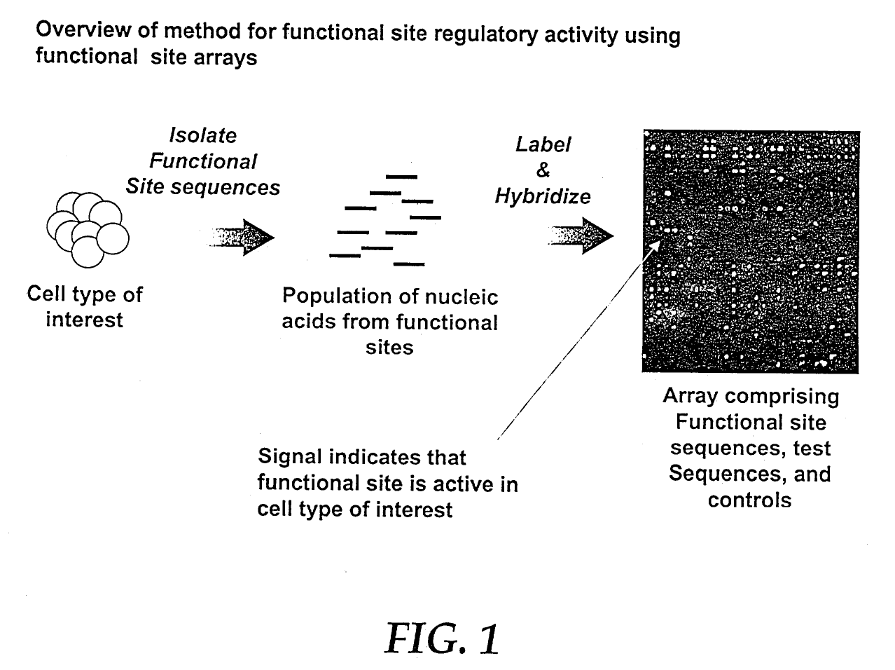

Arrays generally may be of two basic types, passive and active. Passive arrays utilize passive diffusion of sample molecule for chemical or biochemical reactions. Active arrays actively move or concentrate reagents by externally applied force(s). Reactions that take place in active arrays are dependant not only on simple diffusion but also on applied forces. Most available array types, e.g., oligonucleotide-based DNA chips from Affymetrix and cDNA-based arrays from Incyte Pharmaceuticals, are passive. Structural similarities exist between active and passive arrays. Both array types may employ groups of different immobilized ligands or ligand molecules. The phrase “ligands or ligand molecules” refers to biochemical molecules with which other molecules can react. For instance, a ligand may be a single strand of DNA to which a complementary nucleic acid strand hybridizes. A ligand may be an antibody molecule to which the corresponding antigen (epitope) can bind. A ligand also may include a particle with a surface having a plurality of molecules to which other molecules may react. Preferably the reaction between ligand(s) and other molecules is monitored and quantified with one or more markers or indicator molecules such as fluorescent dyes. In preferred embodiments a matrix of ligands immobilized on the array enables the reaction and monitoring of multiple analyte molecules. For example, an array having an immobilized library of functional sites may be tested for binding with one or more putative DNA binding proteins. A two dimensional array is particularly useful for generating a convenient profile that may be imaged, as exemplified in FIGS. 1 through 6.

More recent developments in array manufacture and use are specifically contemplated. For example, electronic arrays developed by Nanogen can manipulate and control sample biomolecules by electrical fields generated with microelectrodes, leading to significant improvement in reaction speed and detection sensitivity over passive arrays (see, U.S. Pat. Nos. 5,605,662, 5,632,957, and 5,849,486). Another active array procedure contemplated in some embodiments is the technology described in U.S. Pat. No. 6,355,491 and issued to Zhou et al. entitled “Individually addressable micro-electromagnetic unit array chips.” This latter technology provides an active array wherein individually addressable (controllable) units arranged in an array generate magnetic fields. The magnetic forces manipulate magnetically modified molecules and particles and promote molecular interactions and/or reactions on the surface of the chip. After binding, the cell-magnetic particle complexes from the cell mixture are selectively removed using a magnet. (See, for example, Miltenyi, S. et al. “High gradient magnetic cell-separation with MACS.” Cytometry 11:231-236 (1990)). Magnetic manipulation also is used to separate tagged functional site sequences during sample preparation in desirable embodiments, before application of DNA to a test array.

Arrays can be used to compare reference libraries as well as profiling based on as little as a single nucleotide difference. The chemistry and apparatus for carrying out such array profiling and comparisons are known. See for example the articles “Rapid determination of single base mismatch mutations in DNA hybrids by direct electric field control” by Sosnowski, R. G. et al. (Proc. Natl. Acad. Sci., USA, 94:1119-1123 (1997)) and “Large-scale identification, mapping and genotyping of single-nucleotide polymorphisms in the Human genome” by Wang, D. G. et al. (Science, 280: 1077-1082 (1998)), which show recent techniques in using arrays for manipulation and detection of sequence alternations of DNA such as point mutations. “Accurate sequencing by hybridization for DNA diagnostics and individual genomics.” by Drmanac, S. et al. (Nature Biotechnol. 16: 54-58 (1998)), “Quantitative phenotypic analysis of yeast deletion mutants using a highly parallel molecular bar-coding strategy” by Shoemaker, D. D. et al. (Nature Genet., 14:450-456 (1996)), and “Accessing genetic information with high density DNA arrays.” by Chee, M et al., (Science, 274:610-614 (1996)) also show known array technology used for DNA sequencing. Array methods for detection of DNA polymorphisms by re-sequencing using multiply redundant oligonucleotide arrays are further described by Patil, N et al. (Science, 294:1719-1723 (2001)) and applied to identification of haplotypes.

Further examples of technology contemplated for use in making and using arrays are provided in “Genome-wide expression monitoring in Saccharomyces cerevisiae.” by Wodicka, L. et al. (Nature Biotechnol. 15:1359-1367 (1997)), “Genomics and Human disease—variations on variation.” by Brown, P. O. and Hartwell, L. and “Towards Arabidopsis genome analysis: monitoring expression profiles of 1400 genes using cDNA microarrays.” by Ruan, Y. et al. (The Plant Journal 15:821-833 (1998)). Additional microarray technologies that may be utilized according to the present invention include, for example, electronic microarrays, including, e.g. the NanoChip Electronic Microarray, which is available from Nanogen, Inc. (San Diego, Calif.) and described in detail in U.S. Pat. No. 6,258,606, “Multiplexed Active Biologic Array”; U.S. Pat. No. 6,287,517, “Laminated Assembly for Active Bioelectronic Devices”; U.S. Pat. No. 6,284,117, “Apparatus and Method for Removing Small Molecules and Ions from Low Volume Biological Samples”; U.S. Pat. No. 6,280,590, “Channel-Less Separation of Bioparticles on a Bioelectronic ChIp by Dielectrophoresis”; and U.S. Pat. No. 6,254,827, “Methods for Fabricating Multi-Component Devices for Molecular Biological Analysis and Diagnostics, and references cited therein, all of which are incorporated by reference in their entirety.

Methods of the invention may further include nanopore technologies developed by Harvard University and Agilent Technologies, including, e.g. nanopore analysis of nucleic acids. Nanopore technology can distinguish between a variety of different molecules in a complex mixture, and nanopores can be used according to the invention to readily sequence nucleic acids and/or discriminate between hybridized or unhybridized unknown RNA and DNA molecules, including those that differ by a single nucleotide only. Nanopore technology is described in U.S. Pat. No. 6,015,714, “Characterization of individual polymer molecules based on monomer-interface interactions,” related patents and applications, and references cited within, all of which are incorporated by reference in their entirety.

In certain embodiments, the invention may employ surface plasmon resonance technologies, such as, for example, those available from Biocore International AB, including the Biacore S51 instrument, which provides high quality, quantitative data on binding kinetics, affinity, concentration and specificity of the interaction between a compound and target molecule. Surface plasmon resonance technology provides non-label, real-time analysis of biomolecular interactions and may be used in a variety of aspects of the present invention, including high throughput analysis of microarrays. Surface plasmon resonance methods are known in the art and described, for example, in U.S. Pat. No. 5,955,729, “Surface plasmon resonance-mass spectrometry” and U.S. Pat. No. 5,641,640, “Method of assaying for an analyte using surface plasmon resonance,” which also describes analysis in a fluid sample, which are incorporated by reference in their entirety.

Microarrays of the invention include, in certain embodiments, peptide nucleic acid (PNA) biosensor chips. PNA is a synthesized DNA analog in which both the phosphate and the deoxyribose of the DNA backbone are replaced by polyamides. These DNA analogs retain the ability to hybridize with complementary DNA sequences. Because the backbone of DNA contains phosphates, of which PNA is free, an analytical technique that identifies the presence of the phosphates in a molecular surface layer would allow the use of genomic DNA for hybridization on a biosensor chip rather than the use of DNA fragments labeled with radioisotopes, stable isotopes or fluorescent substances. A major advantage of PNA over DNA is the neutral backbone and the increased strength of PNA/DNA pairing. The lack of charge repulsion improves the hybridization properties in DNA/PNA duplexes compared to DNA/DNA duplexes, and the increased binding strength usually leads to a higher sequence discrimination for PNA-DNA hybrids than for DNA-DNA.

Arrays of the invention may be prepared by any available means and may contain a variety of different samples, e.g. polynucleotide sequences. In certain embodiments, these polynucleotide sequences may correspond to a set of or substantially all functional sites within a cell. In other embodiments, particular functional sites or genomic sequences may be selected. In one embodiment, sequences of specific genes may be used, such as, for example, sequences associated with a particular cell type, disease state, environmental or other stimuli (e.g. chemical), or developmental stage. In addition, sequences corresponding to a particular region of genomic DNA, such as a gene locus, may be used on an array. Such sequences may cover all or substantially all of a gene locus, and may include coding sequences as well as regulatory and other non-coding sequences.

In certain embodiments, arrays may comprise reduced information sets as compared to arrays comprising substantially all functional sites associated with a cell. Such reduced information sets may be selected based on sequence or genomic location, as described supra, or they may be selected by other means. For example, reduced information set arrays may comprise sequences isolated using particular restriction enzymes and, therefore, may comprise, in specific examples, only 4-cutter-proximal regions or regions proximal to rare cutter restriction sites, which may span large regions.

In one embodiment, repetitive sequences are removed from the arrayed polynucleotides or probes. Repetitive sequences may be removed prior to deposition on an array platform by any means available in the art. For example, repetitive sequences may be adsorbed from a mixture, as described, for example, in Grandori, C. et al, EMBO J 15:4344-57 1996. In another embodiment, repetitive sequences, e.g. genome-specific repetitive sequences may be removed using available bioinformatic algorithms or as described infra. In another embodiment, repetitive sequences may be identified and arrayed. The identification of repetitive sequences then allows them to be removed from profiled produced from the arrays, if desired.

Generally, repetitive sequences may be removed at three levels:

1) Bio-informatically: Algorithms and public engines such as Repeatmasker may be used to identify target sequences which have a high repetitive content. RepeatMasker is a program that screens DNA sequences for interspersed repeats known to exist in mammalian genomes as well as for low complexity DNA sequences. The output of the program is a detailed annotation of the repeats that are present in the query sequence as well as a modified version of the query sequence in which all the annotated repeats have been masked (replaced by Ns). On average, over 40% of a human genomic DNA sequence is masked by the program. Sequence comparisons in RepeatMasker are performed by the program cross_match, an implementation of the Smith-Waterman-Gotoh algorithm (Smit, A F A & Green, P RepeatMasker at http://ftp.genome.washington.edu/RM/RepeatMasker.html). Optionally, identified sequences may be not placed on the arrays.

2) Repetitive sequences may be removed in the hybridization reaction by inclusion of a competitor agent such as Cot1.

3) Repetitive sequences may be removed in the preparation of the probe by doing a subtraction step. For example, Cot1 DNA, or versions of human repetitive elements created by performing PCR with biotinylated degenerate oligos designed to amplify this class of molecules, could be treated with a reagent such as photobiotin, for example, then an excess of this could be hybridized with a non-biotinylated probe population, followed by extraction of all of the biotinylated DNA on DynaI beads. The flow-through would represent repetitive-depleted probe.

Array hybridizations using probes from which repetitive DNA was removed will light up the repetitive control spots on the arrays less intensively than a probe simply made from genomic DNA. Furthermore, targetting the functional sites should be sufficient to ensure a depletion in repetitive elements.

A major advantage of the present invention which is described below is a superior method for the identification and removal of sequences which contribute to false-positive signal via algorithms and methods for predictive genomic hybridization.

A. Methods of Probing Arrays

In addition to providing arrays of functional sites, the invention further provides methods of probing arrays of functional sites, e.g., to determine whether particular functional sites are present or absent within a sample. Such profiling methods have a variety of uses, including, e.g., detection of a disease-associated functional site variant, determining cell or tissue type, and determining whether a drug or other agent affects one or more functional sites. Arrays are typically probed with functional site sequences isolated from a sample. Methods of preparing such probes and probing arrays of the invention include those described in further detail below.

1. Probe Preparation

Probes are typically prepared by marking functional sites using a chromatin modifying agent, isolating or capturing DNA fragments comprising functional sites, and labeling the isolated or captured DNA fragments. These steps may be performed sequentially or one or more may be performed simultaneously.

a. Marking Functional Sites with a Chromatin Modifying Agent

A first step in the preparation of probes (e.g. probes for hybridization to an array of the invention) is to mark functional sites within the sample with a chromatin modifying agent (CMA). Any of the methods and CMAs described supra in the context of identifying and isolating functional sites may be used for probe preparation. In one preferred embodiment, DNAse I is used to mark functional sites by cutting DNA strands at these sites. Examples of other agents and methods that may be used to mark eukaryotic DNAs at functional sites include, for example, radiation such as ultraviolet radiation, chemical agents such as chemotherapeutic compounds that covalently bind to DNA or become bound after irradiation with ultraviolet radiation, other clastogens such as methyl methane sulphonate, ethyl methone sulphonate, ethyl nitrosourea, Mitomycin C, and Bleomycin, enzymes such as specific endonucleases, non-specific endonucleases, topoisomerases, such astopoisomerase 11, single-stranded DNA-specific nucleases such as S1 or P1 nuclease, restriction endonucleases such asEcoR1, Sau3a, DNase 1 or Styl, methylases, histone acetylases, histone deacetylases, and any combination thereof.

As will be appreciated by skilled artisans, clastogens may be used to break DNA and the broken ends tagged and separated by a variety of techniques. Compounds that covalently attach to DNA are particularly useful as conjugated forms to other moieties that are easily removable from solution via binding reactions such as biotin with avidin. The field of antibody or antibody fragment technology has advanced such that antibody antigen binding reactions may form the basis of removing labeled, nicked or cut DNA from a functional site.

In many embodiments, after forming a break or directly binding to the DNA, the affected DNA sequence around the site may be isolated and determined and/or the site mapped to a location in the genome. For example, an agent that forms a covalent bond with DNA may be conjugated to a binding member such as biotin or a hapten. After bond formation, endonuclease may be used to generate smaller DNA fragments. Fragments that contain the marked functional site may be isolated by a specific binding reaction with a conjugate binding member (avidin or an antibody/antibody fragment respectively in this case), for example, on a solid phase that immobilizes the functional site fragments and allows removal of the other fragments.

In another embodiment, following isolation and optional amplification of the DNA segments that flank the sites of CMA modification, the fragments are sub-cloned into a suitable vector, such as a commercially available bacterial plasmid. To effect this, the fragments may be digested with restriction enzymes, cut sites of which have been engineered into the linker regions. Following incorporation into suitable bacterial plasmids, colonies are recovered which contain bacteria in which the plasmid replicates.

Sample preparation begins with chromatin from a sample of cellular material. Preferably, the chromatin is extracted from a eukaryotic cell population, such as a population of animal cells, plant cells, virus-infected cells, immortalized cell lines, cultured primary tissues such as mouse or human fibroblasts, stem cells, embryonic cells, diseased cells such as cancerous cells, transformed or untransformed cells, fresh primary tissues such as mouse fetal liver, or extracts or combinations thereof. Chromatin may also be obtained from natural or recombinant artificial chromosomes. For example, the chromatin may have been assembled in vitro using previously subcloned large genomic fragments or human or yeast artificial chromosomes.

In many embodiments, multiple functional sites are obtained from a eukaryotic cell sample by first extracting and purifying nuclei from the sample as for example, described in U.S. Ser. No. 09/432,576. Briefly, a sample is treated to yield preferably between about 1,000,000 to 1,000,000,000 separated cells. The cells are washed and nuclei removed, by for example NP-40 detergent treatment followed by pelleting of nuclei. An agent that preferentially reacts with genomic DNA at functional sites is added and marks the DNA, typically by cutting or binding to the DNA. In a particularly advantageous embodiment DNAse I is used to form two single strand breaks near each other, and typically within 5 bases of each other. After reaction with functional DNA sites the reacted DNA is, if not already, converted into smaller fragments and the reacted fragments optionally are amplified and separated into a library. Preferably, breaks on both strands within up to 10 base pairs from each other are detected after extraction by cloning one or both sides of the site.

i. Preparation of Soluble Chromatin

In one preferred embodiment, a functional site-enriched sample is prepared by isolating soluble chromatin following treatment with a CMA. Soluble chromatin can be prepared by the action of a CMA on nuclei and fractionated on linear sucrose gradients. Choice of mild treatment conditions causes the soluble chromatin to consist primarily of short fragments released by the action of the CMA on accessible chromatin (i.e. functional sites). Sucrose gradient centrifugation fractionates this material according to mass, and heavier nucleosomal bound DNA fragments are separated from smaller non-nucleosomal DNA. The fraction containing the smallest DNA represents a portion of the genome that is extremely accessible (as it was generated by two digestion events) and not associated with nucleosomes. Both these are properties of functional sites, and, hence, this fractionation procedure produces a functional site-enriched sample. Methods of fractionating chromatin are provided in Examples 15 and 16.

ii. Distinguishing Between CMA and Random Cutting Events

In certain preferred embodiments, several approaches to probe preparation may be employed that have the advantage of distinguishing between sites of chromatin modifying agent (CMA (e.g., Dnasel)) modification within functional sites and sites of random genomic shear during DNA sample preparation. These include approaches employing agarose-embedded nuclei; sucrose gradient fractionation; subtractive hybridization; or a combination thereof.

(a) Agarose-Embedded Nuclei

In certain embodiments, nuclei are encapsulated in agarose plugs to prevent shearing events commonly caused by the processes of nuclear lysis and DNA isolation. When embedded in agarose, the genomic DNA is subjected to fewer mechanical forces during lysis. Prior to recovery from the plugs, the CMA-modified sites are repaired with T4 DNA polymerase followed by A-tailing, in order to distinguish them from any shearing events caused during purification. (See Example 12). Protocols such as that detailed in Example 4 can then be applied to create probes from the sequences demarked by the A-tailed ends.

(b) Sucrose Gradient Fractionation

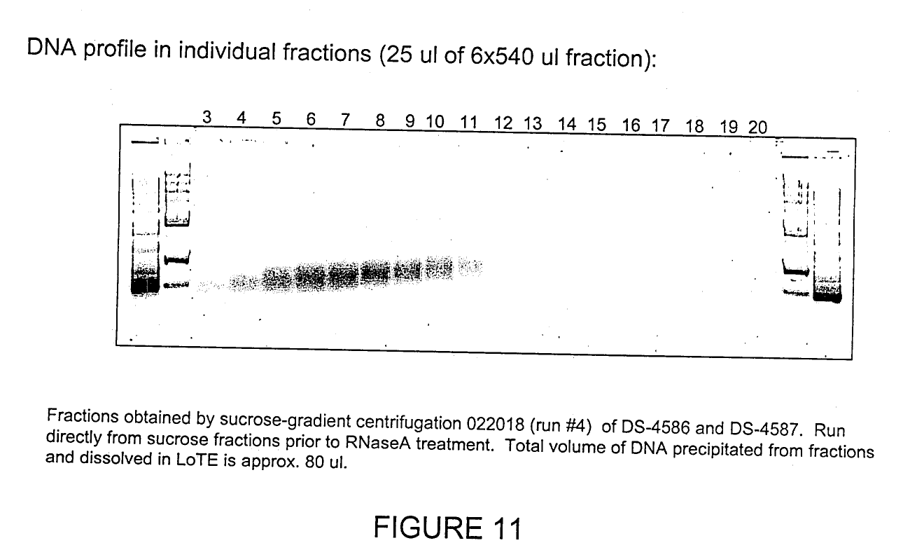

CMA-treated nuclei may be lysed and the released chromatin may be subjected to sucrose gradient fractionation directly, or following DNA purification. (See Example 15). It is expected that chromatin fractions having small size (>200 bp) represent events wherein a CMA has introduced two cut sites within or adjacent to the same functional site (see FIG. 11). In addition, other fractional sizes less than the average size may be prepared by ultracentrifugation, for example, a range of sizes greater than 200 bp and less than ˜10 kb. These fractions will likewise be enriched in DNA fragments with either a single CMA cut site at one end (and a shear at the other) or CMA modification sites from two more widely spaced functional sites.

Sucrose gradient ultracentrifugation may also be employed to effect fractionation by chromatin solubility rather than DNA size, a particularly advantageous approach since functional sites occur preferentially within active chromatin domains of the genome, and these domains display differential solubility under appropriate conditions (See Example 15).

(c) Subtractive Hybridization

Subtractive hybridization is a generic method applied to enrich for sequences present, absent, over-represented, or under-represented in one complex population of DNA fragments when compared to another population. In one context, CMA-treated nuclei (which contain cuts within functional sites) are then subjected to a combination of nucleases to specifically digest the sequences flanking the sites of CMA modification. This material, which represents a population depleted in functional sites (a ‘functional site-minus’ or FS(−) population) can be subtracted from another population, such as fragmented genomic DNA, in order to detect the functional site sequences fully represented in the genomic sample (see Example 13). The method likewise employed can be applied to any differentially enriched fraction containing functional sites including material prepared with sucrose gradient ultracentrifugation, or a DNA fragment populations that has been enriched (through any of the methods disclosed herein) in functional sites from a particular tissue; or from a particular tissue which has been given an environmental stimulus, etc.

b. Isolation/Capturing of Functional Sites

Isolation of DNA after marking and fragmentation may be accomplished by a number of techniques. Exemplary methods include: adaptive cloning linkers that facilitate selective incorporation into a cloning vector or PCR; streptavidin/biotin recovery systems; magnetic beads, silicated beads or gels; dioxygenin/anti-dioxygenin recovery systems; or a variety of other methods. Once isolated (or even before isolation), fragments can be labeled with a detectable label. Suitable detectable labels include fluorescent chemicals, magnetic particles, radioactive materials, and combinations thereof.

Amplification of isolated DNA fragments may be required in the event that the quantities of DNA recovered from this isolation step are insufficient to effect efficient cloning of the desired segments, or simply to produce a more efficient process.

In a desirable embodiment described in Example 1, a biotin-labeled linker is added after formation of cut ends by DNase I and binds to the cut ends. The mixture is digested with one or more restriction endonucleases such as Sau3a or Styl to create smaller fragments and the biotin labeled fragments recovered by a binding reaction to immobilized avidin followed by removal of unbound fragments. An amplification step such as polymerase chain reaction (“PCR”) optionally may be performed. To render the fragments fit for PCR, another linker can be incorporated at the opposite end from that of the biotinylated linker.

Newer variations of PCR and related DNA manipulations such as those described in U.S. Pat. No. 6,143,497 (Method of synthesizing diverse collections of oligomers); U.S. Pat. No. 6,117,679 (Methods for generating polynucleotides having desired characteristics by iterative selection and recombination); U.S. Pat. No. 6,100,030 (Use of selective DNA fragment amplification products for hybridization based genetic fingerprinting, marker assisted selection, and high throughput screening); U.S. Pat. No. 5,945,313 (Process for controlling contamination of nucleic acid amplification reactions); U.S. Pat. No. 5,853,989 (Method of characterization of genomic DNA); U.S. Pat. No. 5,770,358 (Tagged synthetic oligomer libraries); U.S. Pat. No. 5,503,721 (Method for photoactivation); and U.S. Pat. No. 5,221,608 (Methods for rendering amplified nucleic acid subsequently un-amplifiable) are desirable. The contents of each cited patent which pertains to methods of DNA manipulation are most particularly incorporated by reference.

i. Direct Methods

Once the functional site has been cut, either by the action of a nuclease or as a consequence of a secondary reaction which cleaves at the site of a modification introduced into the functional site by a CMA, various methods may be employed to capture the sequences at the cut site. As the sequence recovered is that of the functional site, these methods are referred to as being ‘direct’ and are listed below.

(a) Ligation of Linker

In one embodiment, cut sites are repaired in the isolated genomic DNA by the action of polymerases such as T4 DNA polymerase and blunt ended, and biotinylated linkers are ligated onto these ends using T4 DNA ligase. The DNA is cleaned so as to remove unincorporated linker based upon the size difference as compared to the size of the genomic DNA. At this stage, probes can be made by performing primer extension reactions using an oligonucleotide complementary to the linker.

Alternatively, the size of DNA is reduced either by digestion with restriction enzymes, such as NlaIII, or sonication, to reduce the average size to 500 bp. The fragments are then isolated on strepavidin containing surfaces, such as DynaI beads, and the bulk of the genome washed away. The fraction retained on the beads is then processed as a probe (see Example 17).

Alternatively, after the initial repair step with T4 DNA polymerase, the ends are further altered by the addition of a 3′ A overhang by the action of Taq polymerase. This allows the subsequent ligation of linker to not be blunt ended but to be ‘sticky’, the linker containing a complementary T overhang (see Example 18). The samples are then processed as described above.

(b) Directional Ligation of Linkers

In another embodiment, which is a modification of the above methods, following capture and digestion with a restriction enzyme, a second ligation reaction is performed with a non-biotinylated linker complementary to the exposed restriction site (Example 19). Once ligation has gone to completion, the probe is either retained on the DynaI beads and the unincorporated linker washed away, or advantage is taken of a unique and rare cut site in the first linker to cleave the probe from the beads. The probe can now be amplified exponentially in the PCR reaction using two oligonucleotides complementary to the two linkers.

(c) Biotinylation of Free End by Terminal Transferase;

In another embodiment, the cut sites, which either have been repaired with T4 DNA polymerase or left in their natural state, are treated with terminal transferase in the presence of biotin-ddNTP or a mixture of dNTP:biotin-dNTP to extend the 3′ end of the molecule and so incorporate a biotin moiety. Once cleaned, to remove unincorporated biotin, the average size of the genomic DNA fragments is reduced and the biotin containing molecules captured, typically on DynaI beads. The probe population be prepared by random labeling, degenerate PCR, or any of the common used labeling methods (Example).

Alternatively if the DNA on the beads have been digested with a restriction enzyme a linker can be ligated to those ends and an oligonucleotide complementary to it be used in primer extension reactions.

(d) Creation of Genomic Tags:

A probe population can be generated, as described in (a) above, that is a biotinylated linker is attached to the cut site. This linker contains immediately proximal to the cut site a restriction site for a type IIs enzyme, such as MmeI. Such enzymes cut at sites distal to their recognition site to create genomic tags, in this case of 20 nucleotide length. That length of sequence is sufficient to uniquely place it in the genome the majority of the time and detect its target on an array with high specificity.

Once the immobilized DNA has been cleaved with the MmeI enzyme, a second linker can be ligated to the exposed site (in this case a random two nucleotide 3′ overhang), and this construct cleaved from the DynaI beads by use of a rare restriction site engineered into the first linker to generate a PCR amplifiable genomic tag which can be used in subsequent labeling reactions (Example 8).

(e) Labeling of Free Ends of Agarose Embedded Nuclei

Agarose embedding greatly reduces the amount of breakages introduced into genomic DNA in the course of purification; such breakages constitute a background above the genuine DNAseI cut sites (Example 21). In one embodiment, the nuclei are embedded in agarose immediately after DNaseI digestion, and the DNA is treated in situ according to methods described herein.

(f) Labeling of Free Ends Following Digestion of Nuclei in Manganese-Containing Buffers;

In another embodiment, by increasing the amount of manganese present in the digestion buffer, DNaseI can be made to cut to give a higher proportion of blunt ends or ends with a 1 or 2 nucleotide overhang, as manganese favors a double stranded cutting mechanism. As such, these sites are readily distinguishable from the two sources of background cuts: those due to physical shearing due to preparation of the material which are thought to be staggered; random cutting event of DNaseI in non-functional site sequences, which are likely to be caused by the proximity of two nicks and so also produce a staggered cut, nicking of the DNA (introducing a single stranded break is favored in the presence of calcium/magnesium). Once these sites are generated, they may be labeled as described herein.

(g) Tsc-Ligation Mediated PCR

In another embodiment, the thermostable Tsc ligase is used to add a single-stranded adaptor to a captured, digested functional site sequence (see, e.g., Example 22). The advantage of this step is that Tsc-mediated ligation is a more efficient than blunt-ended or A-tail mediated ligation.

(h) Tsc-Bst Amplification

In yet another embodiment, adaptors are ligated to single stranded genomic tags with Tsc ligase, and the reaction allowed to proceed in order to form linear concatamers and covalent circles, which are templates for Bst polymerase mediated Rolling Circle Amplification (Example 23).

ii. Indirect Methods

Indirect methods refers to approaches whereby a sequence of a proximal marker is isolated and forms the probe. One example is the use of restriction enzyme sites which are close to the CMA cut site. Using these indirect sites has three distinct advantages:

(1) The number of possible targets that the probes can recognize is far smaller than for direct probes, which may hit anywhere within the genome. This decreases the complexity of the target population and allows the efficient design of custom oligonucleotide arrays;

(2) Choice of the restriction enzyme allows selection of the average size of the fragment to which the functional site will be mapped; for example, a rare cutter would allow functional sites to be identified rapidly at low resolution; and

(3) The identification of positives on the array following hybridization is internally controlled; an indirect probe should bind to the targets representing the 5′ and 3′ restriction sites surrounding the functional sites.

The following protocols have been used to create Indirect probes and products:

(a) Creation of Fixed Length Indirect Monotag Populations

In one embodiment, a fixed length indirect monotag population is produced where the site of CMA-mediated cutting is labeled with a biotin, the genomic DNA digested with a restriction enzyme and captured. The linker which is attached to the exposed restriction site has the type IIs restriction site within it, so subsequent digestion releases a genomic tag associated with the restriction site not the DNaseI cut (see Example 24).

(b) Creation of Fixed Length Indirect Monotag Populations Following A-Tailing of DNaseI Cut Sites

An alternative to the protocol described in Example 22 is not to label the DNaseI cut site with a biotinylated nucleotide but instead to add a single dATP 3′ overhang by the action of Taq polymerase. This then allows the efficient ligation of linkers onto this site which can be used to supply a priming site for PCR amplification (see Example 25).

2. Labeling Probes

Labeling of probe populations is achieved by standard methods. In preferred embodiments, this involves performing amplifications (linear or exponential) using synthetically labeled oligonucleotides (containing Cy5- or Cy3-modified nucleotides or amino allyl modified nucleotides, which allow for chemical coupling of the dye molecules post amplification), or rely on direct incorporation of the modified nucleotides during the reaction.

In one embodiment, a DNA fragment subpopulation comprising functional site sequences advantageously may be detected by fluorescence measurements by labeling with a fluorescent dye or other marker sufficient for detection through an automated DNA microarray reader. The labeled fragment population generally is incubated with the surface of the DNA microarray onto which has been spotted different binding moieties and the signal intensity at each array coordinate is recorded. Fluorescent dyes such as Cy3 and Cy5 are particularly useful for detection, as for example, reviewed by Integrated DNA Technologies (see “Technical Bulletin at http://www.idtdna.com/program/tech bulletins/Dark_Quenchers.asp) and as provided by Amersham (See Catalog # PA53022, PA55022 and related description).

As described above, the invention further includes novel methods of tagging or labeling polynucleotides, which are applicable for a variety for purposes, including, e.g. probing arrays of the invention. Specific embodiments and these and related methods of tagging or labeling polynucleotides are described in further detail below, and include the preparation of (1) fixed length direct monotags, (2) fixed length indirect monotags, (3) direct pull down probes, and (4) labeled chromatin probes. The skilled artisan would understand that the exemplary methods described in general throughout and more specifically in the accompanying Examples may be modified in certain respects, according to principles and techniques known in the art, to achieve essentially the same results, and the invention encompasses all such modifications and variations of the described procedures.

a. Fixed Length Direct Monotags

Direct monotags map precisely to either strand of a breakage in the DNA. The breakpoints are typically captured by the ligation of either a blunt or T-tailed linker following repair of the breakage site and Taq-polymerase mediated A-tailing. The linker brings a cutting site for a type IIs restriction endonuclease so it is adjacent to the breakage site. Type IIs restriction endonucleases have the property of cutting a site distal from their recognition site, an example of which is MmeI which cuts 20 nt and 18 nt on the top and bottom strands respectively away from its binding site. This action creates a ‘monotag,’ a snippet of genomic sequence associated with a particular event in the genome, for example, a DNA breakage caused by the introduction of exogenous nucleases. The sequence is of sufficient length to in general allow the majority of them to be mapped uniquely to the genome, or in the context of arrays hybridize specifically to a target sequence.

Some cutting agents will produce breakages with specific features that can be specifically targeted by the linker. Examples of these would include: cutting with DNaseI in the presence of manganese as the divalent cation to produce a predominance of blunt ends; treating nuclei with a restriction enzyme to digest the subpopulation of restriction sites that are accessible in the chromatin (essentially those with fortuitous placements in functional sites) to generate a ‘sticky end’ to which a linker can be ligated. One specific advantage of these approaches is that they do not label breakages which are introduced in a quasi-random fashion in the process of extracting the genomic DNA from the nuclei, this is a considerable source of experimental background.

As the monotags can be derived from strands on either side of the breakage, the system contains an internal control to help screen false positive results. That is, if the probe successfully identifies one target on the array with a certain efficiency, it will be predicted to detect a second target corresponding to the sequence from the other side of the breakage with a similar efficiency.

When that breakage is created by the action of a footprinting reagent, such as DNaseI, hyrdoxyradical reagents or the like, the distribution of monotags can be used to recreate a ‘footprint’ on a specially designed tiling array. The tiling array is so designed that every target polynucleotide, typically each the same size, corresponds to a specific region of DNA, with different targets containing DNA sequences corresponding to shifts of one or more nucleotides relative to each other. For example, a tiling array may be designed such that a target of a 35 nucleotide (or window of some size) stretch of genomic sequence differs from its adjacent target by a shift of a single base pair, so that a series of targets will represent a moving window across the genomic region. If mapping of a lower resolution is required, for example, by using micrococcal nuclease, the digestion pattern of which gives information about the distribution of entire nucleosomes in the chromatin, potentially the gap between the position of the adjacent sequences can be increased; so they are shifted by 5 bp each, or are adjacent but share no overlap, or even are not contiguous sequences. Thus, the invention contemplates overlapping targets with as little as one nucleotide shifts and as large as the entire size of the target, as well as non-overlapping targets. Overlaps may also be of any intermediate size, such as 5 nucleotides, 10 nucleotides, 20 nucleotides, 30 nucleotides, 50 nucleotides, 100 nucleotides, 200 nucleotides, or any intermediate integer value between.

b. Fixed Length Indirect Monotags

As described above, indirect monotags typically map the closest chosen restriction site to the DNA breakage. An example of this procedure is that the breakage site is captured either by direct enzymatic biotinylation, with terminal transferase and biotin-ddUTP, or by ligation of a linker. Following this step, the genomic DNA is cut with a restriction enzyme, NlaIII for example, and a second linker is ligated to that site. It is this linker which contains the restriction site for a type IIs restriction enzme and cleavage with this creates a population of Indirect monotags.

The advantage of this approach is that it allows the experimenter to control the resolution of the experiment and hence the number of data points that need to be collected. While sampling a large space like the human genome with Direct monotags represents 3×10⁹ potential cut sites (to give 1 bp resolution), choosing to map to the nearest 4-cutter restriction enzyme, such as NlaIII, reduces the sample size to approximately 12 million (the predicted number of NlaIII sites) with an average resolution of 250 bp. As for the Direct monotags, the probe population is internally controlled, and the efficiency of detecting NlaIII sites either side of a breakage should be similar. In certain embodiments, Tiling microarrays may be constructed where a 100 kb stretch can be profiled with an estimated 400 oligonucleotide sequences (typically these can be manufactured with 60 nt stretches which correspond to the 25 nucleotides either side of an NlaIII site). Such arrays would allow either de novo discovery of ACEs within that genomic stretch, or, if the sequences are bio-informatically extracted from sequences we have cloned, then the tiling arrays could be used as a validation step for libraries of the invention.

Mapping to the closest NlaIII sites is an efficient way of searching for or validating ACES that are of a similar size. Another application of this embodiment of the invention is the study of larger features within the genome, such as deletions of large genomic (e.g. greater than 0.1 Mbp) within clinical populations. In this scenario, the genomic DNAs are digested with a rare restriction cutter, such as Sse8387I (which produces fragments with an average size of 30 kbp), and the linkers are ligated directly to that site. Cutting from the MmeI site within that linker creates a monotag that can be used to screen and used to make the monotags.

c. Direct Pull Down Probes

In this version of preparing probes, the breakage site is again either enzymatically labeled (as described above) or ligated to a biotinylated linker. Following a purification step to remove unincorporated biotin substrates, the genomic DNA is cut with a restriction enzyme. The majority of the genome will be contained within the simple restriction fragments and as they have not been labeled with biotin will not be captured on a separation system, such as paramagnetic beads coated with strepavidin. The biotinylated ends, marking the breakage sites, are captured, and this fraction is then taken forward to be labeled in order to create a probe population.

Modifications can be made to the process whereby in place of the restriction digest of the genomic DNA it is randomly broken, either by physical shearing, sonication or treatment with non-specific or low-specificity cutters of naked DNA, such as DNaseI. These protocols have advantage that they are rapid and reproducible.

d. Probes Made from Labeling of Chromatin Fractions

Sucrose gradient centrifugation or other preparative methods can be used to isolate discrete fractions of treated genomic DNAs according to their mass. These fractions can then be labeled directly to produce probes or used as a source for monotag populations. The rationale for this approach is that it is more likely that smaller fragments will contain a genuine cutting site for an ACE than not, i.e. it consists of two random background cuts. Certainly, the ability to remove the vast majority of high molecular weight DNA considerably reduces the background due to isolated random breakages (either caused by the action of the exogenously added enzyme or shearing due to handling).

A variety of different targets and probes have been described and may be used according to the invention, in any combination. In certain embodiments, targets and/or probes may be of a fixed length, while in other embodiments targets and/or probes may be of variable length. Accordingly, in specific embodiments, combinations of the invention include fixed target and fixed probe lengths, variable target and fixed probe lengths, fixed target and variable probe lengths, and variable target and variable probe lengths.

3. Binding of Probe to Array

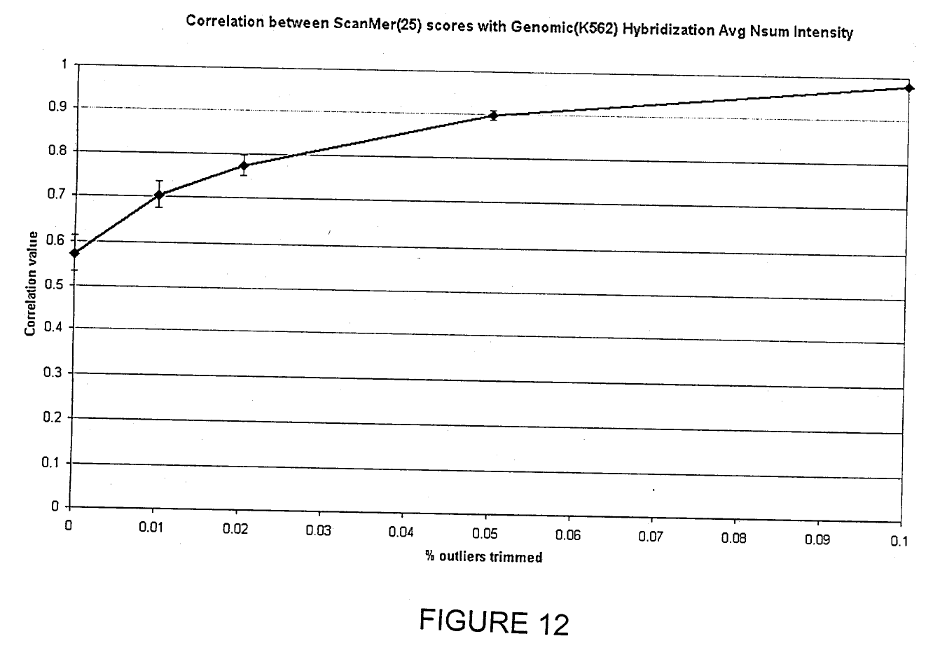

Probe populations are incubated with arrays of functional site binding moieties under conditions appropriate for sequence-specific binding. As understood by the skilled artisan, such conditions vary and depend upon the nature of the arrayed functional site binding molecule, e.g. polypeptide or polynucleotide. In preferred embodiments of the invention, arrays comprise polynucleotides comprising functional site sequences, or fragments, complements or variants thereof. DNA-protein and nucleic acid-nucleic acid binding conditions are known in the art and are described, for example, in U.S. Pat. No. 6,171,794 and references cited therein. Exemplary hybridization conditions are described in Example 4. The skilled artisan would understand that the permissible ranges and other conditions (% formamide, etc.) may be varied. Example 27 describes the process of procuring data from an array experiment. Example 28 describes correlation of scanmer scores and genomic hybridization scores shown in FIG. 12.

4. Construction and Use of Genomic Indexes and their Application to Predictive Genomic Hybridization

The completed draft sequences of the human and various model organisms have enabled post-genomic computational methods that heretofore were either impossible or inefficient. With the exponential growth of available data rapid and novel techniques are necessary to locate and retrieve genomic DNA and protein sequences. The standard algorithms embodied by FASTA and BLAST while providing proximity inexact matching of a query and target sequence can only deliver matches that are close to the query sequence, and rely on filtering techniques to eliminate alignments that have low probability of similarity.

The availability of genome-wide data sets enables a new approach based on a theory of genomic ‘indexing’. Databases of significant size such as microarray data, genetic maps, expression databases and other data types may be benefit from an indexing approach that would enable nearly instantaneous retrieval of query sequences. In the case of significant downstream computation requirements such performance time enhancements are essential. Indexing methods may also be applied in the context of comparative genomics allowing for rapid sequence comparison between organisms. Additionally data mining techniques may benefit form up front indexing as opposed to real time sequential searching.

In order to facilitate such rapid information retrieval and to enable new types of heretofore impossible or inefficient analyses, the invention provides a very general system—termed MerCator—for genomic indexing of either DNA or protein sequences. This system is embodied in an efficient application of a novel indexing theory. The method described by this theory enables exact indexing of genome sequences with efficient storage, and subsequently rapid search and retrieval of exact and near exact query sequences against a target sequence.

a. A Genomic Indexing Method

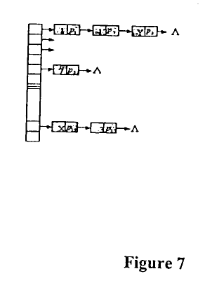

The MerCator method has two phases: Indexing and Retrieval. The index phase is performed once per target genomic dataset and it proceeds as follows: A linear scan of a target genome is performed encoding each k-mer, an oligonucleotide consisting of k consecutive nucleotides. Each k-mer is binary encoded in a natural manner using two bits per nucleotide if genomic DNA is encoded, and 2′ bits where/is sufficiently large so that the necessary number of nucleotides can be recovered, if protein sequences are considered. For example, in the case of genomic DNA, the sequence TACGT is encoded as 1100011011, the binary representation of decimal 795. Next a hash table is constructed of length equal to length 4^{l} where each entry corresponds to the decimal representation of a binary encoded k-mer. During the indexing phase, each time a given k-mer is found the position and chromosome of that k-mer are hashed to the appropriate bucket and that information is added to a linked list. A graphical illustration of this data structure is illustrated in FIG. 7.

The data structure depicted in FIG. 7 in its current form is insufficient for most real world genomic applications due to the following space limitations. During the indexing phase of MerCator shorter k-mers can be indexed provided that only those occurring with lower frequency counts are stored. For smaller k<10, the number of k-mers occurring in the human genome is too large to be of practical use for all but a small number of mers. On the other hand for k>12, the hash table cannot be constructed in RAM on a typical high-performance computing device that utilizes a 32-bit processor. The problem is improved only somewhat by moving to the larger scale architectures of 64-bit or 128-bit and potentially higher, as rapid retrieval and higher information content sequences will continue to be necessary. The need for a general method is clear.

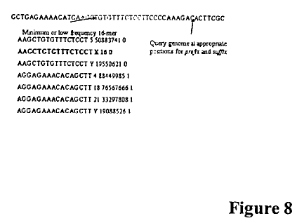

To overcome these issues, two specific conceptions were formed. The first concerns the length of the hash table itself, the second the length of the linked lists in the data structure. As the main objective of MerCator is accurate and rapid localization, the k-mers that are being indexed must be sufficiently long to enable quasi-unique placement in the genome or placement a relatively small number of times. The actual data structure used is a generalization of the one displayed in FIG. 8 and uses methods from suffix trees to efficiently store all the mers indexed within a desired range.

The above arguments indicate that for the purposes of genomic localization in MerCator the size of k on which to index is critical. Smaller k yields sequences that occur too often in the genome, whereas longer k yields nearly unique sequences but places too much computational overhead on the system. It was discovered that the best compromise is to choose k in some range over which localization is optimized to within some confidence value, and this is the combined goal of both the indexing and retrieval steps of the ScanMer algorithm.

This process may be formalized using the following notation:

A ‘unique mer’ is defined to be an oligonucleotide sequence occurring exactly once in a target genome,

A ‘quasi-unique mer’ is defined to be such a sequence occurring less than some bounded number of times M in the target genome.

Let Q be a query sequence.

Let T be a target.

By ‘localization of Q in T’ we mean identification of the unique position of Q in T or a null pointer if Q does not occur in T.

By ‘approximate localization’ we mean the query sequence Q can be located in T with mismatch of up to a fixed number b of base pairs of T.

This process is thus repeated for a range of short mers. This total range is not critical but must contain the range starting from the shortest quasi-unique mers, those occurring less than some fixed number of times in the genome, and bounded above by the mer size necessary such that the probability that the k-mer is unique is greater than a fixed amount. This data structure is efficiently implemented using standard techniques from the theory of suffix trees.

b. MerCator indexing Algorithm

Let G be a target genome. Choose mer size k such that there exists a predetermined probability κ(k) of k-mers that are quasi-unique in G. Choose mer l such that the probability that the l-mer is unique is λ(l). Let Iⱼ denote the construction of the ScanMer data structure described above for a mer of size j. Let P denote the probability of unique localization or approximate unique localization of a query Q in G.

Index I={Iⱼ:k≦j≦l} such that P>P* with confidence (1−α) 100% for 0≦α≦1.

Utilizing this strategy one insures unique localization of a query string Q against a target sequence T with given probability and confidence.

c. Search and Retrieval Using MerCator

Once a genomic sequence database has been indexed for a given k-mer, retrieval of k-mers becomes a simple lookup for k in the range of application of the ScanMer indexing algorithm. However, there is subtlety in the alignment and localization of arbitrary mers against the target genome. To gain intuition into this process let us consider the searching for a longer sequence of genomic DNA 50 base pairs. A useful observation is the following: If a long mer has genomic significance, then it most likely occurs a limited number of times in the genome. Probabilistically speaking this means that the mer must contain a considerably shorter mer that occurs only a relatively small number of times. If we can find shorter mer, and if a ScanMer index exists for this shorter mer, we may leverage the database of chromosomes and positions to accurately localize the larger mer. For example, suppose during the indexing phase a database was built only by indexing 16-mers. Then during the search phase we perform a binary search of the input long mer in an attempt to locate a lower frequency 16-mer. Once the lower frequency 16-mer is found, using each of its positions from the database we check the prefix and suffix of that 16-mer with respect to the input mer for appropriate matches.

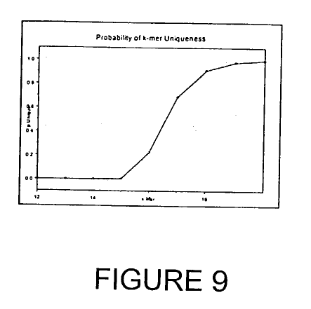

Central to this concept is the probability of uniqueness of a given k-mer in the genome. Through standard arguments using a Poisson arrival rate the uniqueness of a k-mer can be shown to follow a curve as shown in FIG. 9. During the retrieval and localization phase of the algorithm ScanMer tracks this curve from more unique to less unique is search of an optimal positioning marker.

d. Generalized Alignment and Short Inexact Matches Using MerCator

The MerCator system immediately yields a variety of tools that are useful for PCR primer design and microarray analysis. As many query sequences match only weakly with their target, it is natural to raise the issue of finding short inexact matches. An extension of the basic MerCator system allowing for inexact matches can be performed by searching for the occurrence of short exact matches within a target sequence and/or by varying the nucleotides of the query sequence individually. We may formalize this process as follows.

e. MerCator Alignment Algorithm:

Let R(mᵢ) denote the genomic frequency count from a database retrieval of a mer of size mᵢ constructed during the ScanMer indexing phase described above. Set an upper bound for quasi-uniqueness M. This number should be less than or equal to the value used for quasi-uniqueness during the indexing phase. Let k and l be the minimum range of mer sizes indexed as determined by the ScanMer indexing phase. Let Q be a query mer and T a target sequence in genome G. Finally let γ be a percentage rate for correct matches in T deemed to represent success. If γ=1 then only exact matches are accepted if γ=0.75 matches are valid with up to 75% correct alignment.

For j=I down to k do {

Locate by binary search a mer mⱼ ⊂ Q having R(mⱼ) < M in T

For each position in T determined by mⱼ ⊂ Q do{ //

attempt to match prefix and suffix boundary ends

Form prefix p and suffix s determined by Q − mⱼ.
$If ( match ( p \& s in T ) > γ - \frac{j}{\vert T \vert} ) return success ;$
Else continue; I

}

}

The intuition of the MerCator alignment algorithm may be described as follows: A near-optimal mer mⱼ⊃Q is first located from the index set which is quasi-unique in T. Each of these positions is retrieved from the indexed database of T. This determines a certain fraction of the required match percentage

$$γ - \frac{j}{\vert T \vert} .$$

The remaining prefix and suffix of the query Q are matched against T to obtain the full γ match.

The MerCator alignment algorithm described in this section enables a highly efficient and general procedure for query/target genomic or proteomic alignment allowing for exact and inexact matching.

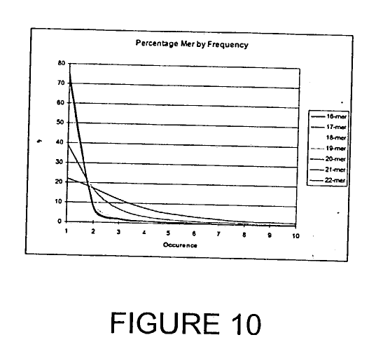

For example, direct calculation based on the MerCator indexing results enables near exact calculation to within 99% confidence of the total frequency counts for any query mer size against the human genome. This seemingly daunting and practically intractable computational task may be performed via MonteCarlo simulation in about 2 hours on a modest size multiprocessor cluster using the MerCator algorithm. Exact frequency distribution of 16-22 mers as calculated using the ScanMer indexing system are depicted in FIG. 10.

Due to the prior indexing step, fast database retrieval, and leveraging the localization of the short exact match mers, MerCator significantly out performs conventional algorithms such as BLAST or FASTA. Other algorithms based on short oligonucleotide sequences such as BLAT leverage non-overlapping 1-mers and are restricted in their performance on shorter query sequences. It was found that ScanMer outperforms by approximately a factor of 10 in speed of query over each of these systems, and in fact any such available system.

f. Predictive Genomic Hybridization—The ScanMer System

A surprising discovery made in the practice of the MerCator invention was the finding that an application (henceforth referred to as ScanMer) could be developed that enabled prediction of hybridization efficiencies of genomic DNA fragments to oligonucleotides or other collections of nucleic acids. This problem—which is termed here ‘Predictive Genomic Hybridization’—has heretofore proven insurmountable and intractable using the known art in molecular biology and computational science and combinations thereof.

Moreover, another application was discovered for the ScanMer system, namely its great utility in the design of microarrays, and particularly of oligonucleotide microarrays. In this system unique localization is each probe of genomic DNA is essential and was discovered to be strongly correlated with hybridization. In previous attempts to solve the predictive hybridization problem, researchers have used a measure of simple repeat content as determined by the RepeatMasker utility. RepeatMasker (developed by A. Smit and P. Green) screens DNA sequences in FASTA format against a library of repetitive elements and returns a masked query sequence ready for database searches as well as a table annotating the masked regions. RepeatMasker has provided an effective way of identying repeatable elements in the genome such as SINES, LINES, microsattelites, CpG islands, and other highly occurring elements.

Our laboratory analyses have shown that as a predictor of genomic hybridization, RepeatMasker performs poorly or not at all, since it masks elements that are quasi-unique, and fails to mask certain repeatable sequences.

Through the practice of the MerCator system, an algorithm was discovered that provided an accurate and predictive genomic hybridization score. This algorithm is embodied in the ScanMer system.

g. The ScanMer Algorithm Enables Predictive Genomic Hybridization

To enable predictive genomic hybridization an algorithm was discovered that encapsulates a scoring function that serves as the basis of measuring average repeatable content in the genome that is available for differential hybridization:

Let M denote a long mer of length |M| and m a shorter mer of length |m|. By r(m) we denote the MerCator alignment score described above. Then the average ScanMer ‘score’ is then given by

$${S}_{M} = \frac{1}{\vert M \vert} {\sum }_{i = 0}^{⌊ \vert M \vert / m ⌋} {\sum }_{j = 1}^{\vert m \vert} {α}_{j} r ( {m}_{i \vert m \vert + j} )$$

where the coefficient αⱼ denotes a weighting factor that accounts for correlations between overlapping mers of length |m|. Intuitively, the ScanMer score captures the following. A long mer M is divided into small mers m whose score is given by the average value of repeat content across the range M. As each mer m overlaps subsequent m−1 mers shifting downstream, a correction factor is necessary to remove the frequency contribution determined by the correlation of subsequent mers m. A proper average is done over the full target mer M.

The ScanMer score S_{M} was found to be an accurate measure of genomic hybridization to nucleic acids immobilized on microarray systems. FIG. 12 depicts the striking correlation between actual genomic hybridization signals and predicted signals based on the ScanMer score both before and—more dramatically—after removal of outliers according to standard statistical techniques (see Example 28).

Moreover, an additional novel application was discovered to the design of successful primers for the Polymerase Chain Reaction.

B. Methods of Using Functional Site Arrays

In preferred embodiments of the invention a set of at least 10 functional site sequences and/or locations obtained from a sample are combined to form a profile of the sample. Typically an array is made that can detect the sequences and generate a data profile indicating at least a) the presence or absence of each sequence or functional site in a sample or b) the relative abundance of functional sites from a sample. It was discovered that “detection” of (i.e. determination of the presence and/or relative abundance of) at least some of the functional sites of a sample as a group profile on an array can reveal useful characteristics of the sample. Such characteristics include, for example, whether the sample contains a DNA break that increases the risk of particular malignancies or has a highly expressed region with respect to a normal state.

In another embodiment, a sample is processed to determine functional site usage and a profile is obtained from binding reactions between nucleic acid sequences obtained from the sample and other nucleic acid references. Advantageously either the reference nucleic acids or the sample nucleic acids are first bound in an array and the array exposed to the other set. In an embodiment at least 10, more preferably at least 100, 1000, 10,000, or even more than 20,000 reference nucleic acids are used in this embodiment.

In yet another embodiment a sample is processed to generate nucleic acids corresponding to sequences of functional sites and the nucleic acids identified by sequencing, mass spectrometry and/or another method. Profile results obtained advantageously are compared to known values.

Yet another embodiment of the invention provides a master organism reference library that contains a large collection, e.g., greater than 100, greater than 10,000 or greater than 25,000 functional site sequences representative of the organism. In one embodiment, the library substantially contains all possible assayable functional sites of a cell. The phrase “substantially contains” in this context means at least 10% and preferably at least 50% of all possible functional sites, including every site that can be found in one situation (cell type, cell morphology, or other condition) or another. Preferably “substantially contains” refers to at least 75% of all possible functional sites, and more preferably refers to at least 90%, 95% and even at least 99% of all sequences and/or site locations. In an embodiment such library is made by mapping functional sites from at least 3 different cell types of an organism and more preferably 4, 5, 6, or even more than 10 types of different cells, and compiling all of the different functional sites into a “organism specific” set of functional sites. One version of a library includes sequences corresponding to each functional site. Yet another version of the library includes position information of each functional site. Either or both versions of data are very useful tools for diagnostic tests and other studies.

Yet another embodiment is a cell type specific reference library that “substantially contains” all functional sites of that specific type of cell. Another related embodiment is a library prepared from a cell or cells treated with an external stimuli, such as a drug or environmental stimuli, for example. External stimuli may include any compound, such as drugs, small molecules, hormones, cytokines, etc., and any other types of treatment or stimulation, such as changes in environmental factors, e.g. temperature, pressure, or atmosphere, and including radiation, for example. The term “substantially contains” in this context means at least 10% and preferably at least 50% of all functional sites that are active under one or more conditions experienced by that cell type. More preferably, “substantially contains” refers to at least 75% of all possible functional sites, and even more preferably refers to at least 90%, 95% and even at least 99% of all sequences and/or site locations. By way of example, a human cell line was found to contain approximately 30,000 functional sites, when examined in late log stage of growth.

In certain embodiments, libraries and arrays of the invention may contain functional sites associated with one or more specific genes or genetic loci, including, e.g. genes known to be associated with diseases or other disorders.

Many uses of the invention arise from the ability to generate, manipulate and analyze large amounts of information through libraries and their use in microarrays to provide information. Arrays generally are made and used by a variety of methods that can be discussed in terms of i) preparation of arrays; ii) sample preparation and conversion into fragment libraries, iii) manipulating the fragments by, for example, amplifying and cloning them, and iv) profiling libraries (i.e. either the entire set of prepared fragments or a subset of them) by detection on arrays.

C. Methods of Functional Site Profiling

As described above libraries may exist in silico as DNA sequences or in vitro as physical elements that contain DNA. In other embodiments libraries are profiled on arrays. Data obtained from large assemblages of library elements are useful for many purposes. In principle, two or more arrays are prepared under similar conditions with one array acting as a control or reference for the other(s). For example, alteration of expression induced by a test compound such as a drug candidate may be determined by creating two arrays, one that corresponds to cells that have been treated with the test compound and a second that corresponds to the cells before treatment.

Differences in array data profiles can reveal which functional sites are affected by the test compound. A functional site may be more sensitive to CMAs in the presence of the drug, as seen by more abundant hits at that functional site during the nuclei incubation/reaction step leading to a stronger functional site signal in a profile. A functional site may be found less sensitive to CMAs if, in comparison to a no-drug control, a weaker signal was produced for that functional site spot in the array. In another example, an array profile obtained from a malignant tissue sample may be compared with an array profile obtained from a control or normal tissue sample. An inspection of the functional site differences between the arrays may reveal a genetic cause in the disease or a genetic factor in the disease progression.

A functional site profile may be as simple as a small set of 6, 7, 8, 10, 10 to 25, 25 to 100, or 100 to 500 functional site. The procedures and materials illustrated in “Cystic fibrosis mutation detection by hybridization to light-generated DNA probe arrays.” by Cronin, M. T. et al. (Human Mutation, 7:244-255 (1996)), and “Polypyrrole DNA chip on a silicon device: Example of hepatitis C virus genotyping.” by Livache, T. et al. (Anal. Biochem. 255:188-194 (1998)) are particularly contemplated for determining differences between a reference sequence or library sequence and that obtained from a sample. These documents are specifically incorporated by reference and illustrate the knowledge of skilled artisans in this field.

In another embodiment an array generates data that reveal functional site copy number. As will be readily appreciated, some functional sites are more sensitive to CMAs than others for a given cell state and this character can be seen as a higher copy number, or (where appropriate) a greater detection signal compared to another functional site or reference sample. According to an embodiment of the invention, the relative copy numbers of one or more functional sites are compared to a reference or set of references to determine a relative activity of the functional site.

Without wishing to be bound by any one theory of this embodiment of the invention, it is believed that functional site profiling in this manner often yields a more accurate determination of gene regulation than measuring transcribed mRNA or a protein product of a gene because “hypersensitivity” itself is a more direct measure of whether a regulatory system is on or off. In contrast, mere quantitation of a transcription or translation product generally reflects more variables and may be less tightly associated with the biochemical operation of the corresponding regulatory unit. One embodiment of the invention is an improvement in previous diagnostic and quantitative tests for gene regulation wherein one or more functional site s and/or a functional site profile is determined by an array and correlated with a particular protein function or other biological effect.

Another embodiment of the invention is a set of primers corresponding to a library of functional site s and which can form an array. Preferably the library contains at least 10, 100, 250, 500, 1,000, 5,000 or even more than 10,000 primers that correspond to specific functional sites. In an advantageous method a library of functional site specific primers are used to selectively amplify or detect functional site sequences corresponding to a particular desired profile. A library profile may be as small as a set of 5 or 10 functional site sequences. In this case 5 or 10 primers with sequences corresponding to the desired functional sites may be used with a DNA sample to selectively amplify those functional sites for further analysis.

The library profiling and comparison techniques of the invention are useful for discovery of drugs that interact with regulatory mechanisms mediated by one or more functional sites. A respective embodiment directly screens for drugs by exposing a microarray of functional site sequences to potential drugs. Another embodiment scores the effect of a chemical on an intact nucleus by exposing the nucleus to the drug and then deriving a library of functional sites from the treated nucleus. Representative techniques and materials useful in combination for this embodiment are found in “Selecting effective antisense reagents on combinatorial oligonucleotide arrays.” by Milner, N. et al. (Nature Biotechnol., 15:537-541 (1997)), and “Drug target validation and identification of secondary drug target effects using DNA microarray.” by Marton, M. J. et al. (Nature Medicine, 4:1293-1301 (1998)).

While many embodiments of the invention concern profiled information from arrays, the fragment libraries and derivatives of them are independently valuable tools. A fragment library prepared by marking and separating out functional sites from chromatin contains valuable information that may be extracted and used in a variety of forms. For example, the fragments can be sequenced and their profile information entered into a computer or other data base for comparison in silico with one or more reference libraries. In addition, an functional site fragment can be used to identify and isolate one or more coding regions with which the functional site sequence is associated. Moreover, the fragments may be cloned and used for drug discovery via one or more screening techniques described herein and apparent to an artisan of ordinary skill in view of the instant disclosure. Isolated fragments may be cloned by any of a number of techniques using any number of cloning vectors. Exemplary techniques include: introduction into self-replicating bacterial plasmid vectors; introduction into self-replicating bacterophage vectors; and introduction into yeast shuttle vectors.

Generally, the fragment library may be converted by an array manipulation in silico or in vitro into other valuable libraries by a variety of techniques. For example, members of the library having highly repetitive sequences may be deleted from computer memory by pattern matching and removal of matched sequences. Highly repetitive sequences and/or other undesirable sequences/sites such as those found by random breaks during DNA isolation. Such fragment libraries, either as computer data base set or as physical DNA containing sets of vessels, molecules, plasmids, cells or organisms, are valuable items of commerce. For example, a library obtained from tissue of a patient with a particular disease will represent a snapshot of the active functional site profile associated with the disease and has significant value for drug discovery and for diagnosis. Both a computer based data set library and physical embodiments of that set such as a library of clones has great utility and may be sold for a variety of purposes.

In view of the various array-based library screening methods described herein, it will be appreciated by the artisan of skill in the art that the disclosed methods for generating functional site profiles, and the functional site profiles so obtained, provide valuable sources of novel and important biological information. Indeed, a number of important advantages of the present invention stem from the ability to readily compare functional site profiles in biological samples., e.g., at different developmental stages, across different cell types, in different disease states, and/or in response to candidate therapeutic compounds, etc.

For example, in one embodiment, the present invention provides a method for profiling cell or tissue samples functional site profiles are first generated from one or more test samples and the profiles so obtained are then compared to a reference profile in order to identify differences in functional site activity between the two samples. The identification of one or a plurality of functional sites that is characteristic of a given disease state relative to a healthy control state, for example, provides important diagnostic information about the disease state. In one example, functional site profiles are generated in accordance with the present invention for at least two samples or sets of samples, one representing healthy control tissue and the other representing diseased human tissues, in order to identify functional site activity that is altered in the disease state. The invention thus provides methods for identifying functional site profiles that are associated with, and thereby diagnostic for, a disease state, such as cancer. For example, functional site profiles can be generated for a collection of samples, e.g., breast cancer samples, and compared to a suitable reference profile such as a profile generated from normal healthy tissue of the same type from which the cancer sample was derived, i.e., normal breast tissue. Alterations in activity of an individual functional site sequence, or in a pattern of functional site activities, can be readily detected and quantitated by the array profiling methods described herein to identify a “signature” profile of functional site activity that is characteristic of, and preferably diagnostic for, the disease. The activity of individual functional sites and/or the activity of a group or pattern of functional sites, is thus correlated with the occurrence of the particular disease state. In this way, tissue profiling identifies functional site sequences and groups of sequences that have utility in methods for the diagnosis and/or monitoring of the disease state with which the functional sites are associated, as well utility in the screening and discovery of drugs that modulate the functional site activity related to the disease.

In another embodiment, the invention provides methods for screening and identifying test compounds for their ability to modulate the activity of an individual functional site or a group or coordinated pattern of functional sites. In one embodiment, as discussed briefly above, two or more arrays can be prepared under similar conditions with one array acting as a control or reference for the other(s). For example, alteration of expression induced by a test compound such as a drug candidate may be determined by creating two arrays, one that corresponds to cells that have been treated with the test compound and a second that corresponds to the cells before treatment.

Differences in array data profiles can reveal which functional site s are affected by the test compound. A functional site may be more sensitive to CMAs in the presence of the drug, as seen by more abundant hits at that functional site during the nuclei incubation/reaction step leading to a stronger functional site signal in a profile. A functional site may be found less sensitive to CMAs if, in comparison to a no drug control, a weaker signal were produced for that functional site spot in the array. In another example, an array profile obtained from a malignant tissue sample may be compared with an array profile obtained from a control or normal tissue sample. An inspection of the functional site differences between the arrays may reveal a genetic cause in the disease or a genetic factor in the disease progression.

In another embodiment, the arrays and methods of the invention are used for systematic and simultaneous identification of regulatory variants and their corresponding hypersensitivities (i.e. functional impact of variant). For example, this approach can be taken when a tissue containing a regulatory variant, such as a SNP, has been discovered it can be used to generate probes for screening by array profiling. If the position and nature of the regulatory variation is known relative to a nuclease cutting site, typically DNaseI, or to a restriction site, an indirect probe can be made from the tissue. The probe can be designed so as to contain the altered sequence. A collection of molecules could also be designed containing the versions of the regulatory sequence with and without the variation. The conditions of hybridization can be made so specific that matches between probes and targets only occur when they are homologous. In this way it can be shown whether a variation, which may occur as a heterozygous state, led to the failure of functional site formation. In still further embodiments, functional site regulatory variants can be screened, for example, for association with a particular disease state, for altered responsiveness to one or more test compounds relative to the corresponding wild type functional site sequence, and/or for association of a particular pharmacogenetic variant with a particular array signature.

In yet another embodiment, microarray based hybridization as described herein, or similar technologies available in the art, are used for the relatively high resolution profiling of a discrete genetic locus. For example, one can design oligonucleotides and primers to generate uniformly sized PCR products, which can be used to create collections of sequences which when either arrayed on a microarray, or some similar platform, allow the screening of contiguous or overlapping stretches of sequences covering genomic locations, e.g., a genetic locus of interest. Typically the genomic locations are chosen to include a gene locus, that is the entire sequence of a gene of interest and surrounding sequences in which it is likely that some or all of the regulatory elements of that gene are included. The amount of sequence covered on a single slide depends on a number of factors, but where necessary multiple slides can be used so there is no theoretical limit to the extent of sequences queried in this manner.

The length of the target DNA (the DNA that is immobilized) can vary from as small as 20 nucleotide of unique sequence in an oligonucleotide, though 35 or 60 nucleotides are more common. When oligonucleotides are used sequences are chosen which represent both strands of the DNA. PCR primers can also be designed to generate typically 250 bp or 500 bp products as target molecules. The sequences are generally designed so that they are either contiguous or adjacent molecules have some extent of overlap, the most extreme example of which is where with the oligonucleotide targets each sequence is shifted by a single base pair. Certain sequences, such as highly repetitive sequences, can be excluded from the target sequences. The platform selected in the certain embodiments will be those in which the area of the microarray and the maximum number of spots it is possible to array.

In another embodiment, the arrays and methods of the invention are used for phylogenetic regulatory profiling. A large number of functionally active genetic elements would be expected to be conserved between different species, the more the closer the species are in evolutionary terms. Thus, according to another embodiment, probing a collection of these elements identified in one species, such as human, with a probe population constructed from a second species, such as mouse, would identify which of the elements have homologues in the probing population. This analysis of homologues can be extended to other species and also by comparing, amongst other attributes, the patterns of regulation of the homologues by creating probes from permissive and non-permissive tissues. These approaches have the advantage that nothing need be known about the genomic sequence of the organism from which the probe population is being made. Other methods rely on obtaining large amounts of sequence with which to perform multiple alignments in order to detect regions of conserved DNA, the biological activity of which then needs to be defined in a separate assay (conservation of sequence per se is not a foolproof marker of activity).

In another embodiment, functional site isolation and profiling in accordance with the present invention is amenable to array-based analysis for use in the discovery and analysis of underlying networks of genetic regulation. The use of such data is advantageous compared to cDNA expression data as the present methods enable monitoring the event or events which determine expression and, moreover, allows for analysis of large numbers of data points in an efficient and high throughput fashion.

In another embodiment, the methods and arrays described herein are used in the context of chemogenomic profiling. Chemogenomics represents the discovery and description of all possible compounds that can interact with any protein encoded by the human genome. Broadly, it now appears to mean taking a combinatorial approach to screening protein targets by family/class and as such represent s a vast collection of closely related compounds which need to be screened in a high-throughput mode. Thus in another embodiment, functional site arrays described herein may be used to both confirm the pathway of action of any active molecule and to potentially detect any unexpected changes induced in the array.

In one specific embodiment of chemogenomic profiling, probes are prepared by cleaving genomic DNA with a chemotherapeutic agent, and profiles are thus established for different chemotherapeutic agents or different cells. It is known in the art that different cancers sometimes respond quite differently to a chemotherapeutic drug. Chemogenomic profiling of the response of different cancers to different chemotherapeutic agents permits the identification of cancers that may be more or less amenable to treatment by any given chemotherapeutic agent and can therefore be used to screen patients prior to treatment. For example, genomic sites targeted by a particular drug and associated with a favorable clinical outcome may be identified and then used to screen patients before treatment with the drug or to identify other cancers that may be amenable to treatment with the drug, since such cancers may display a similar chemogenomic profile. Furthermore, chemogenomic profiling according to the invention allows the identification of genomic locations that are modified in different tumors or by different drugs, as indicated by their particular profile. More specifically, insight may be gained into the disease process or the mechanism of action of the drug by examining chemogenomic profiles generated according to the invention. For example, profiles for a particular cancer may be examined before and after treatment with a drug known to be therapeutically effective to identify genomic locations that are modified in the tumor. Such locations are likely involved in the disease process.

In another embodiment, the methods and arrays described herein are used in the context of methylgenomic profiling. For example, probes are developed which are sensitive to, in the first instance, the presence of cytosine methylation in the CpG dinucleotide. It is known that this modification plays a role in genomic regulation. Other modifications can also be targeted with this technology and would include adenine methylation in plants or other organisms where it is found to occur and cytosine methylation where it occurs in different sequences, an example of which is C^{m}CWGG. Probing can be performed on a collection of sites, such as those contained in an array according to the present invention, or a locus profile, to for example examine changes in methylation patterns on induction of a gene, or on a genomic level, using a panel of microarrays or similar platform.

In yet another embodiment, the arrays and methods of the present invention may be used to evaluate deletions in genomic regulatory sequences. Two illustrative approaches are briefly described that can address this important question of how the loss of genetic material is associated with the onset of disease. For example, arrays described according to the present invention can be probed with a genomic DNA sample prepared from a diseased cell line or tissue and compared with a similar genomic reference probe (labeled with a different color) to determine and identify the functional site sequences that are either absent, or over represented, in the diseased state. This strategy of using functional sites as genetic markers for this type of analysis offers the advantage over other approaches of identifying sequences which are most likely to be important in genomic regulation. In another example, one can generating probes from genomic DNA which map the occurrence of certain restriction sites. That is by use of cutters such as SseI8387I which on average cuts every 30 kb within the human genome to create indirect probe populations it is possible to perform hybridization with a custom tiling array containing all the sequence information immediately adjacent to this site. Spots on the array which show a change in signal, relative to a non diseased genomic probe created in a similar fashion, can be taken to represent where a change in the copy number of that particular restriction fragment has taken place in the diseased genome. Using this approach, it will be possible to estimate whether a deletion event is either hetero- or homozygous and also to determine the numbers of any duplication event. The choice of enzyme, its cutting frequency and properties (some enzymes show methylation sensitivity) will determine the resolution at which these genomic alterations can be mapped.

In another embodiment, the invention provides methods for comprehensively assessing the epigenetic status of chromatin in a sample by multimodality probing of array regulatory sequences. For example, the Chromatin Immunoprecipitation assay allows the recovery of DNA sequences from eukaryotic nuclei by antibody recognition of epitopes present on associated proteins within the nucleoprotein complex. This approach advantageously provides a means to recover DNA on the basis of either the enzymatic modifications of the histone proteins (referred to as the histone code and including, but not limited to, histone H4 and H3 acetylation, histone H3 methylation, and histone H1 phosphorylation) or the presence of specific proteins (be they members of the basal transcriptional machinery or certain transcription factors) or post-translationally modified versions of such proteins (which can be modified in a similar way to histone proteins). Once antibody recognition has been used to isolate the nucleoprotein complex the recovered DNA can be used to make one or more classes of probes, such as those described herein, e.g., pull-down probes, direct monotag probes or following restriction an indirect monotag probe.

Hybridization experiments useful in accordance with this embodiment may include the following. In one example, ChIp pull-down probes will be used to query a standard array spanning some genomic sequences, typically contiguous 250 bp fragments spanning 50-100 kb of a gene locus, in order to determine the patterns of an epigenetic modification and correlate it with previously determined expression and structural data. In another example, a reiteration of the above experiment is carried out with DNA prepared by performing the ChIp experiments with a comprehensive collection of antibodies with specificity for all known and some novel histone modifications in order to generate a detailed description of the ‘histone code’ across a locus. In another example, by preparation of the ChIp-material from a range of transcriptionally permissive and non-permissive cells and tissues or following the effects of the histone code following environmental stimuli or induction of the gene with specific chemicals, it is possible to deduce the in vivo sequence of events which control or contribute to transcriptional regulation. Finally, another example involves assaying the effect of a class of potentially therapeutic molecules which are designed to modify the activities of the histone modifying enzymes not only on a gene of interest (as with locus profiling) but also by scanning large sections of the genome by creating in parallel an indirect monotag probe and hybridizing to appropriate tiling arrays.

In another embodiment, multimodality profiling is provided as an alternative to performing sequential screens with DNA reagents prepared by one of the discussed selection techniques (such as sensitivity to nucleases or chemicals, selection of nucleoprotein complexes by antibodies etc.). For example, one such approach can involve performing multiple selections in parallel, for example perform a ChIp protocol with an antibody raised against histone H4 acetylation and then reselecting that population with a second antibody raised against a different modification. Similar combinations of ChIp selections with nuclease/chemical sensitivity selections can be performed, as can selection based upon the methylation status of any preselected population.

### EXAMPLES

The following specific examples are provided to illustrate embodiments of the invention, and should not be viewed as limiting the scope of the invention.

### Example 1

### Preparation of DNA Microarrays Containing Functional Sites

Primer pairs were designed to allow amplification of approximately 500 bp PCR products from human genomic DNA. Following two rounds of amplification, where in the second one-hundredth volume of the original PCR reaction is used as a template, the PCR products are purified (using Millipore Multi-screen PCR purification plates), quantified (A260) and their concentration established to be between 50 ng/ul-150 ng/ul. The size of the PCR products is checked by agarose gel eletrophoresis before the microarrays are printed (in 50% DMSO) onto mirrored slides (RPK0331, Amersham) using Amersham's Lucidea Arrayer. The PCR products are crosslinked to the slides with 500 mJ, using Stratagene's Stratalinker. The slides are stored desiccated until use.

### Example 2

### Preparation of DNA that Contains One or More Single-Stranded or Double-Stranded Cleavage Sites within Domains Defined by Functional Sites

K562 cells were grown to confluence (5×105 cells per cubit milliliter as assayed by hemocytometer). Nuclei were prepared from a suitable volume (e.g., 100 ml) and nuclei were prepared as described (Reitman et al MCB 13:3990). Briefly, Nuclei were resuspended at a concentration of 8 OD/ml with 10 microliters of 2 U/microliter DNaseI [Sigma] at 37° C. for 3 min. The DNA was purified by phenol-chloroform extractions and ethanol precipitated. The DNA was repaired in a 100 microliter reaction containing 10 microgram DNA and 6 U T4 DNA polymerase (New England Biolabs) in the manufacturer's recommended buffer and incubated for 15 min at 37° C. and then 15 min at 70° C. 1.5 U Taq polymerase (Roche) was added and the incubation continued at 72° C. for a further 10 min. The DNA was recovered using a Qiagen PCR Clean-up Kit and the DNA eluted in 50 microliter of 10 mM Tris.HCl, pH 8.0

### Example 3

### Isolation of DNA Fragments Associated with Functional Sites

DNA was mixed in a 100 microliter reaction volume containing 50 pmol of PS003 adapter (created by annealing equimolar amounts of oligonucleotides 5′ biotinylated PS003f and 5′ phosphorylated PS003r, to create an adapter containing a NotI site) and 40 U T4 DNA ligase (New England Biolabs) in the manufacturer's recommended buffer for 16 h at 4° C. The sequences of these oligonucleotides are: 5′ Bio_TTATGCGGCCGCTATGTGTGCAGT PS003F and 3′ GAATACGCCGGCGATACACACGTC PS003R.

The reaction was incubated at 65° C. for 20 min before the DNA was isopropanol precipitated in the presence of 0.3 M NaOAc and after ethanol washing resuspended in 20 microliter TE buffer (10 mM Tris.HCl, 1 mM EDTA, pH 8.0). The DNA was digested in a 50 microliter reaction volume containing 20 U Hsp92 II (Promega) in the manufacturer's recommended buffer by incubation at 37° C. for 2 h, after which a further 20 U of enzyme was added and the incubation continued for 1 h and then heated to 72° C. for 15 min. The DNA was captured on M-270 DynaI beads as per manufacturer's instructions.

The beads were finally washed in 200 microliter of ligation buffer before capture and resuspension in a 100 microliter reaction volume containing 50 pmol of Hsp adapter (made by annealing equimolar amounts of oligonucleotides fHsp and rHsp) supplemented with 6 U T4 DNA ligase (New England Biolabs) in the manufacturer's recommended buffer and incubated at 16° C. for 16 h. The reaction was heated to 65° C. for 15 min prior to capture of the beads. The beads were washed in 1×NEB3 buffer (New England Biolabs) and then resuspended in a reaction volume of 100 microliter of the same buffer supplemented with 40 U NotI (New England Biolabs) and incubated for 37° C. for 1 hour with occasional mixing. Afterwards, the beads were captured and the supernatant retained. The beads were washed once and the resultant supernatant combined with the first and isopropanol precipitated in the presence of 20 microgram glycogen and 0.3 M NaOAc. After ethanol washing, the DNA was resuspended in 10 microliter of 10 mM Tris.HCl, pH 8.0.

It will be clear to those skilled in the art that fragments isolated by the procedure above, or modifications thereof, may be used as reagents for the isolation or identification of genomic DNA segments that flank the site of DNA modification by combination with separately prepared population of genomic DNA that has been fragmented by other methods.

In the case of this specific embodiment/example, it is desirable to perform an amplification step prior to subcloning. It is anticipated that such a step may be required in some, but by no means all instances of the application of the process of the invention, as mentioned above. To perform amplification of the recovered DNA fragments prior to cloning, PCR may be employed or other methods of amplification, such as RCA (Rolling Circle Amplification) or versions of it. To render the fragments fit for PCR for example, another linker can be incorporated at the opposite end from that of the biotinylated linker mentioned above. A PCR amplification was then carried out.

To confirm that the DNA segments isolated with the above procedure contain ACE regions that would be expected in an erythroid cell line such as K562, the products were probed for the presence of nuclease functional sites known to be present in this cell type.

### Example 4

### Labeling of DNA Fragments Associated with Functional Sites

Two μg of DNA were diluted into a volume of 24 μl with water and 20 μl of 2.5× Random Primers Solution (Invitrogen, constituent of BioPrime Labeling Kit) and the mixture heated to 95° C. for 5 min. The mixture is cooled on ice for 5 min before 2 ml dNTP solution (consisting of 5 mM Promega's dATP, dGTP, dTTP and 1 mM dCTP) and 3 μl of either 1 mM dCTP-Cy3 or dCTP-Cy5 (Amersham) and 1 μl of 40 U/ml Klenow (Invitrogen). The mixture was incubated at 37° C. for 2.5 h before being stopped by the addition of 5 μl of 0.5 M EDTA. The probes were purified on Qiagen QIAquick columns and eluted in 100 μl of EB. The amount of incorporation was calculated by reading the absorbance at 550 nm (for Cy3) and 650 nm (for Cy5) and probes were mixed at a dye molar ratio of 4:1 (pmol Cy3:pmol Cy5). Typically 200 pmol of Cy3 labeled probe was used and 50 pmol Cy5.

### Example 5

### Preparation and Labeling of Control DNA Fragments

Genomic DNA was isolated from K562 nuclei which had not been treated with a nuclease (1 ml of nuclei with an A₂₆₀ of 8 OD/ml) and had been subsequently digested with NlaIII to completion and the DNA purified using a Qiagen Dneasy column. The concentration of the DNA was corrected to 150 ng/μl. These probes were labeled with Cy3.

### Example 6

### Hybridization of Functional Site-Associated and Control DNA Fragments to Functional Site-Containing DNA Microarrays

The calculated amounts of probes were mixed and dried down in the dark. The paired probes are resuspended thoroughly in 8.5 μl 4× Hybridization buffer (Amersham, #RPK0325) and 8.5 μl water and then mixed with 17 μl formamide and vortexed. The mixture was heated at 95° C. for 3 min then cooled by spinning at 13K for 2 min. 30 μl of this hybridization solution was dispensed in a thin line across a slide and spread evenly over the surface by laying on of a coverslip and incubated at 42° C. for 16 h in a humid and darkened hybridization chamber.

The slides were washed in the dark with gentle agitation. The washes used were 5 min at 37° C. in Wash 1 (1×SSC, 0.2% SDS), two 5 min washes at 37° C. in Wash 2 (0.1×SSC, 0.2% SDS) and two 5 min washes at room temperature in Wash 3 (0.1×SSC). The slides were air-dried and scanned immediately using Packard Biosciences ScanArray 4000.

### Example 7

### Overview of Processes

An overview of a representative process is illustrated in FIG. 1. This figure shows how the structural integrity of functional sites within a sample may be determined in a two step process: A probing reagent is created and compared to a query population. To create the reagent, cells are treated by a procedure developed to isolate and label a population of DNA fragments from the genome that is enriched in those structurally formed functional sites or a functional subset of them, such as transcriptional enhancers, or a structural subset, such as methylated sequences. In this example, these DNA fragments are used as a probe to hybridize against a population of sequences on a microarray. Those sequences may be a set of previously characterized functional sites, may physically span a section of the genome or be a large enough combination of oligonucleotides to allow discretion of complex binding patterns. Following analysis the presence and intensity of the signal reflects the extent to which that particular functional site has formed within that population of cells.

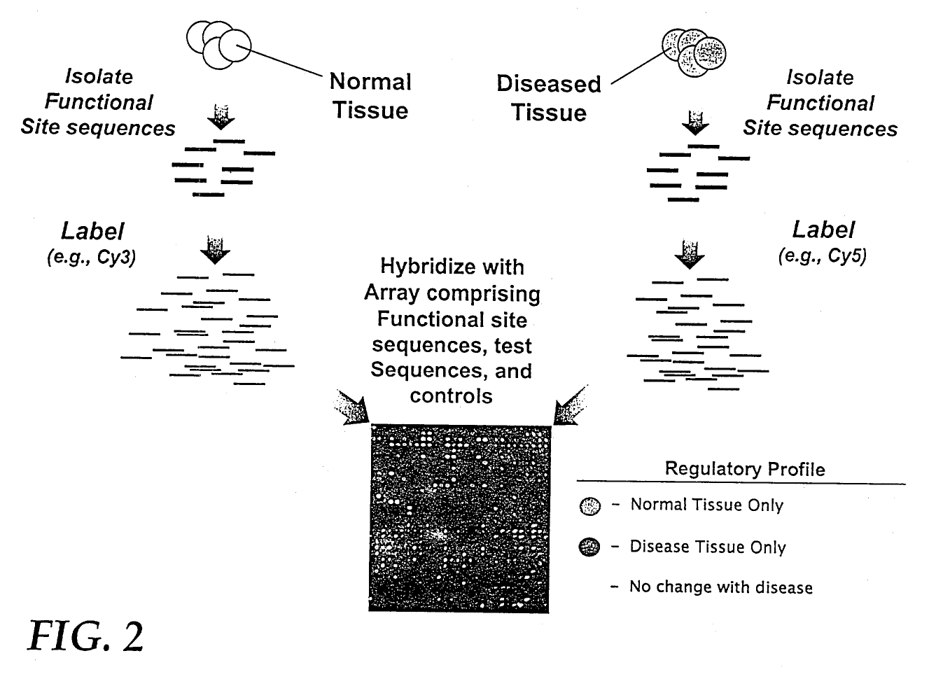

Alternatively, the process may be carried out in parallel using two different markers in order to reveal a differential expression pattern. This process may be employed to increase the signal-to-noise ratio as illustrated in FIG. 2. Here, the sensitivity and accuracy of microarray hybridization will be maximized by comparing the signal of two populations of probes generated by the same procedure but isolated from a treated and non-treated population. In this example, the probe labeled with Cy3 was enriched for functional sites whilst the Cy5-labeled probe will contain functional sites at the same frequency as they occur in the genome. As the probes are generated the same way, they will share similar physical characteristics, such as length and labeling efficiency. Therefore, the ratio of intensity seen on a co-ordinate in the array will accurately reflect enrichment of the sequence in one of the probing populations. In this example, a structurally formed functional site in the cell population would give rise to a green (Cy3) spot, while an unformed site would be yellow (equal amounts of Cy3 and Cy5 bound) or red (Cy5).

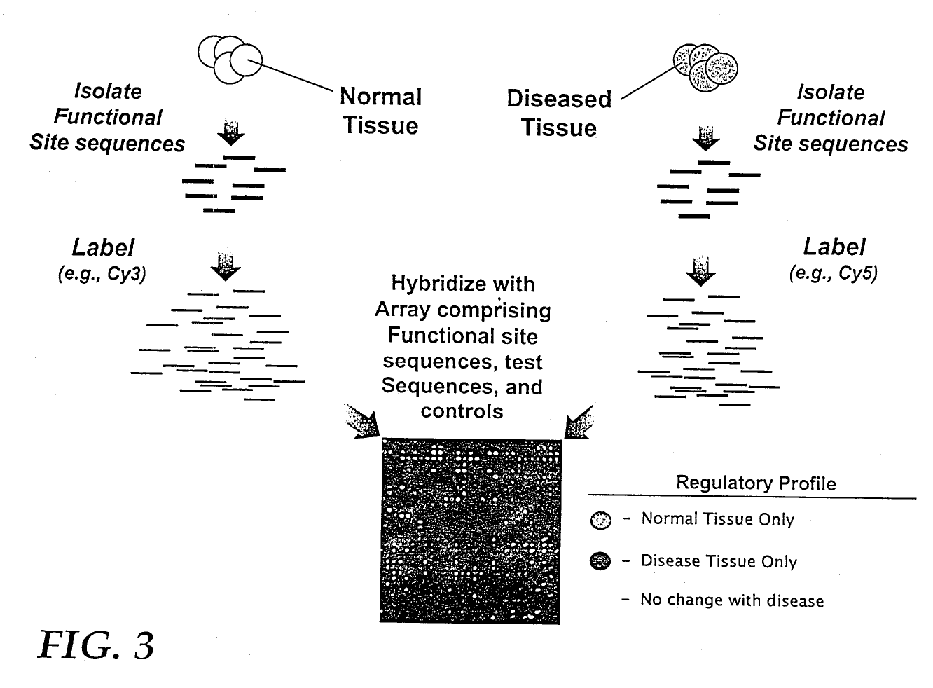

Several further additional applications of the invention are illustrated in FIGS. 3 through 6. These include:

i. Differential profiling of regulatory elements (i.e., between two different cell populations). An overview of this process is illustrated in FIG. 3. FIG. 3 shows how the technology can be used to examine the dynamic nature of functional site formation. In this example, two cell types are treated with a similar procedure to generate from each a differently labeled probe population enriched in functional sites. As in FIG. 2, the probes will have similar physical characteristics which allows their direct comparison. Hence, a functional site formed in one tissue but not the other will label its spot predominately red or green, while those formed in both tissues will color yellow. The exact ratio of Cy3 to Cy5 will provide information about the relative abundance and activity of that functional site in the tissues. Any functional sites that are absent from both tissues will not be lit up on the array.

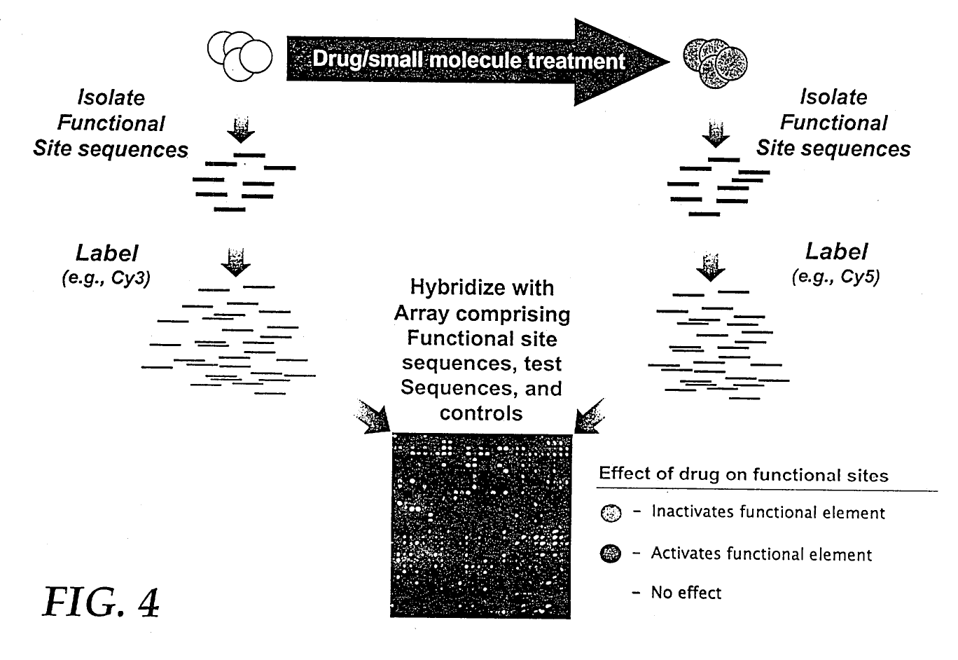

ii. Screening for compounds or treatments that impact the regulatory element activity profile. An overview of this process is illustrated in FIG. 4. As seen here, profile changes may be monitored to show changes in the pattern of functional sites in response to stimuli. Comparative hybridization, as described in FIG. 3, can be used to determine, in this example, which functional sites are induced or repressed by treatment with a drug or small molecule. A probe population is prepared from a reference population of untreated cells and compared to that of a differently labeled probe from the cells following treatment following hybridization to the microarray.

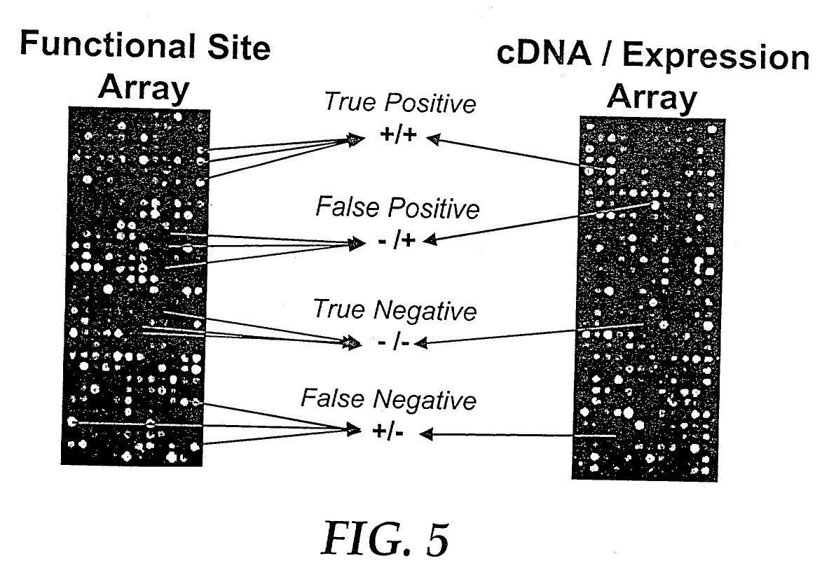

iii. Correlation of regulatory element activation patterns with gene expression patterns to construct regulatory network maps. An overview of this process is illustrated in FIG. 5, which establishes a correlation between functional site and expression data. Parallel analysis of gene expression, as detected by use of expression arrays, and functional site structural integrity will give information about functional sites implicated in transcriptional control of specific genes. Such correlation will also enable improved quality control for conventional expression arrays.

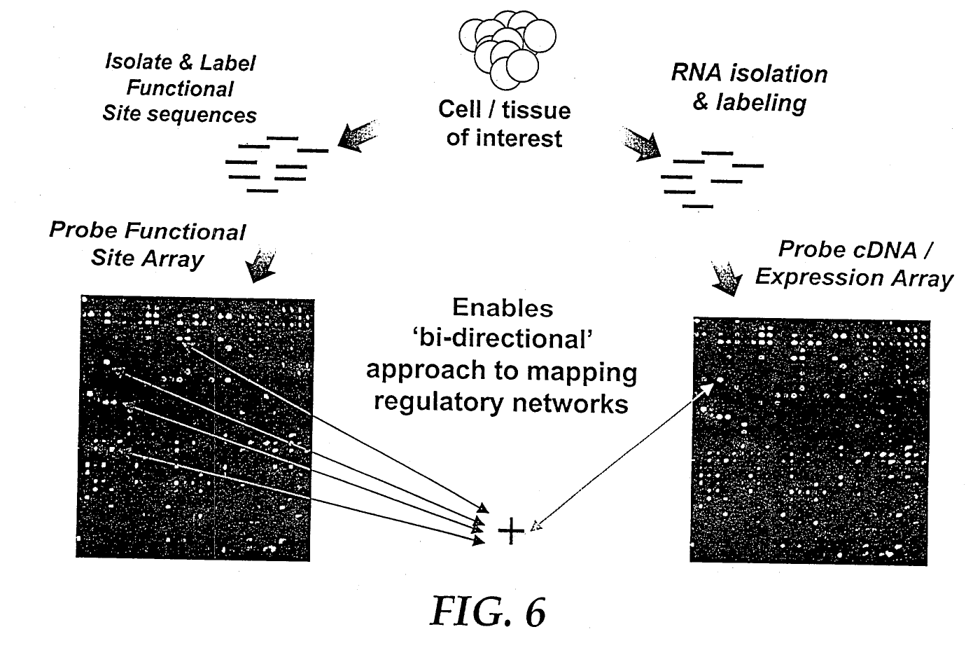

iv. Correlation of regulatory element activation with gene expression to provide a powerful biological quality control assay for gene expression arrays. An overview of this process is illustrated in FIG. 6.

### Example 8

### Method for the Production of Fixed Length, Direct Monotag Probes for Hybridization to Ace Microarrays

Direct monotag probes for use in accordance with the present invention were generated according to the following protocol.

A. Genomic DNA was First Cleaned Using a Centricon YM30 Column, According to the Following Protocol:

1. Wash Centricon 30 column through with 400 ul TE pH 8.0 or water

2. Spin 10 mins @ 6000 rcf

3. Add g.DNA (10-15 ug) and spin 10 mins @ 6000 rcf

4. Wash 2×500 ul TE pH 8.0 and spin 15 mins each

5. Elute with 200 ul TE (10Mm Tris 0.2Mm EDTA)

6. Let column sit 30 mins @ 37° C.

7. Invert column and spin 3000 rcf for 3 min

8. Check DNA on 0.8% agarose gel and take OD.

B. Blunting and Tailing of the DNA was Performed According to the Following Protocol:

1. Combine 100 ul cleaned gDNA & 11.0 ul 10×PCR buffer+MgCl₂

2. Incubate @ 65° C. for 10 mins

3. Place on ice and add Master Mix

4. Prepare Tailing Mix as follows:

4.0 ul 1 Ox PCR buffer x MgCl2

2.0 ul dNTP's 10Mm

1.0 ul T4 DNA polymerase

1.0 ul Taq polymerase

30.0 ul H2O

5. Add 40.0 ul tailing mix to DNA and incubate @ 37° C. for 15 mins

6. Remove and incubate @ 72° C. to add A's for 15 mins

7. Clean on PCR clean-up column to remove enzymes. etc.

8. Elute in 150.0 ul EB

C. Ligation of Adapter 1 was Performed Using the Following Primers and Protocol:

5′Biotin-
CTC TGG CGC GCC GTC CTC TCA CGC GTC CGA CT
GAG ACC GCG CGG CAG GAG AGT GCG CAG GCT G-5′

1. Prepare Ligation Mix as follows:

143 ul cleaned gDNA

16 ul 10× ligase buffer

1.0 ul Adapter 1 @ 50 pmol/ul

*0.5 ul T4 DNA ligase NEB 400 U/ul

2. Add ligase in 1× ligase buffer+0.5 ul ligase 10 ul per tube

D. Cleaning Up on Ligation to Remove Unincorporated Adapter was Performed According to the Following Protocol:

1. Clean using PCR column as per manufacturer's instructions (Qiagen)

2. Elute with 500 ul TE preheated to 55° C.

3. Leave for 10 mins at 37° C.

4. Spin and retain 1.0 ul to run on QC gel

5. Clean again using Centricon 100 column—prepare column as before by eluting through with 400 ul TE/water to remove glycerol.

6. Spin at 200 rcf

7. Load on elute from PCR column (500 ul)

8. Spin at 500 rcf for 15 mins (retain elute)

9. Wash x 2500 ul TE and spin again at 500 rcf for 15 mins (filter should look fairly dry at this point)

10. Add 100 ul of 10 Mm Tris Ph 8.0

11. Allow to sit 30 min to re-dissolve DNA bound to column

12. Carefully invert column and collect in clean tube by spinning at 3000 rcf for 3 min

13. Run 5.0 ul of first flow through and 1.0 ul of collected sample on QC gel (0.8% Agarose)

14. Run for 60 min, stain and scan.

E. Digest I with Mme1 was Performed as Below:

1. Prepare digestion mixture as follows:

100 ul Adapter DNA

11.5 ul 10×Mme1 buffer

1.0 ul SAM at 50 uM final conc.

2.0 ul Mme1

1.0 ul BSA

F. Binding to Beads was Performed According to the Following Protocol:

1. Re-suspend 10 ul M271 and capture

2. Wash×2 in 1×BB

3. Re-suspend in 115 ul 2×BB and add beads to Mme1 digested DNA

4. Allow to bind at room temperature on rocker for 30 mins

5. Capture and retain s/nat for QC gel

6. Wash x 2 in wash buffer (10 Mm Tris pH 8.0, 50 Mm Nacl, 1 Mm EDTA)

G. Digest 2 with Mme1 was Performed According to the Following Protocol:

1. Wash in 50 ul 1×Mme1 buffer

2. Capture and re-suspend in 30 ul digest

3.0 ul 10×NEB4 buffer

3.0 ul SAM (1/64 dil)

22.0 ul H2O

2.0 ul Mme1

0.5 ul BSA

3. Digest for another 30 mins at 37° C.

4. Capture on beads and repeat digestion once more by re-suspending beads in digestion mix

5. Incubate 37° C. for another 30-40 mins

H. Labelling Monotags was Accomplished as Followed:

1. The beads were then used directly in a labelling reaction using an oligo labelled with Cy5 or Cy3.

5′Cy5/3-CTC TGG CGC GCC GTC CTC TCA CGC GTC CGA CT

2. The following mixture is added to 1 μl of the beads:

10 ul PCR buffer

4.0 ul labelled oligo (5 pmol/μl)

2.0 ul 10 mM dNTPs

0.5 ul hot start Taq

83.5 ul water

3. The reaction mixture is cycled on the following program: 95° C. for 2 min, 93° C. for 15 s, 60° C. for 15 s, 72° C. for 15 s; x 30; 72° C. for 2 min, 4° C. on hold

### Example 9

### Method for the Production of Fixed Length, Indirect Monotag Probes for Hybridization to Functional Site Microarrays

Fixed length, indirect monotag probes were prepared by following the following protocol:

A. Digestion of Genomic DNA with Sse8387I was Performed as Follows:

Sse8387I is an 8-cutter enzyme, insensitive to methylation, which recognizes and restricts the site 5′-CCTGCA GG-3′ and has an estimated 10⁵ sites in the human genome is used as follows.

1. Digest two aliquots of 20 μg each of clean genomic DNA from either a cell line (K562) or primary tissue

2. Phenol-chloroform extract

3. Ethanol precipitate in the presence of 1/10 volume of 3 M NaOAc and 2 volumes ethanol

4. Wash and resuspend in 10 μl water

B. Ligation of Linkers

1. The following oligonucleotides were annealed to give two sets of linkers:

PS_Af (5′ Biotin)
CTC TGG CGC GCC GTC CTC TCA CGC GTC CGA CTG CA
PS_Ar (5′ Phosphate)
GTC GGA CGC GTG AGA GGA CGG CGC CCC AGA GC

PS_A Linker

MluI MmeI
5′-Biotin CTC TGG CGC GCC GTC CTC TCA CGC GTC CGA
CTG CA C GAG ACC GCG CGG CAG GAG AGT GCG CAG GCT G

2. Set up the following two ligations:

4 μl 10× T4 DNA ligase buffer (Promega);

1 μl T4 DNA ligase (3U/ml);

10 μl Sse8387I-digested DNA (10 μg);

1 μl PS_Linker A or B (50 pmol/μl);

24 μl water.

3. Incubate overnight at 4° C.

4. Clean reaction on DNeasy column to remove unincorporated primers

5. Resuspend in 10 μl EB buffer

6. Ethanol precipitate in the presence of 1/10 volume of 3 M NaOAc and 2 volumes ethanol

7. Wash and resuspend in 10 μl water.

C. Digestion with MmeI was Accomplished as Follows:

1. Set up the following digestions on both samples:

3 μl 10×MmeI buffer (Gdansk);

10 μl Sse8387I-digested DNA+Linker A (10 μg);

1 μl MmeI (2 U/μl);

16 μl water.

2. Incubate at 37° C. for 3 hours

3. Capture on M-270 DynaI beads

4. Wash 10 μl DynaI beads twice with 100 μl 2× Binding buffer, resuspend beads in 30 μl 2× Binding buffer and combine with 30 μl of MmeI-digests. Allow to bind for 30 mins at room temperature with mixing

D. Labelling Monotags

1. The beads were then used directly in a labelling reaction using an oligo labelled with Cy5 or Cy3

5′Cy5/3-CTC TGG CGC GCC GTC CTC TCA CGC GTC CGA CTG CA

2. The following mixture was added to 1 μl of the beads:

10 μl PCR buffer

4.0 μl labelled oligo (5 pmol/μl)

2.0 μl 10 mM dNTPs

0.5 μl hot start Taq

83.5 μl water

3. The reaction was cycled on the following program: 95° C. for 2 min, 93° C. for 15 s, 60° C. for 15 s, 72° C. for 15 s; x 30; 72° C. for 2 min, 4° C. on hold

### Example 10

### Method for the Production of Variable Length, Direct Pull Down Probes for Hybridization to Functional Site Microarrays

The Cy5 probe was prepared as follows. Nuclei were prepared from K562 cells and resuspended at a concentration of 8 OD/ml with 10 μl 2 U/μl DNaseI [Sigma] at 37° C. for 3 min. The DNA was purified by phenol-chloroform extractions and ethanol precipitated. The DNA was repaired in a 100 μl reaction containing 10 μg DNA and 6 U T4 DNA polymerase (New England Biolabs) in the manufacturer's recommended buffer and incubated for 15 min at 37° C. and then 15 min at 70° C. 1.5 U Taq polymerase (Roche) was added and the incubation continued at 72° C. for a further 10 min. The DNA was recovered using a Qiagen PCR Clean-up Kit and the DNA eluted in 50 μl of 10 mM Tris.HCl, pH 8.0. The DNA was mixed in a 100 μl reaction volume containing 50 pmol of adapter A (created by annealing equimolar amounts of oligonucleotides 5′ biotinylated PSAf and 5′ phosphorylated PSAr) and 40 U T4 DNA ligase (New England Biolabs) in the manufacturer's recommended buffer for 16 h at 4° C. The reaction was incubated at 65° C. for 20 min before the DNA was isopropanol precipitated in the presence of 0.3 M NaOAc and 10 μg glycogen and after ethanol washing resuspended in 20 μl TE buffer (10 mM Tris.HCl, 1 mM EDTA, pH 8.0). The DNA was digested in a 50 μl reaction volume containing 20 U Hsp92 II (Promega) in the manufacturer's recommended buffer by incubation at 37° C. for 2 h, after which a further 20 U of enzyme was added and the incubation continued for 1 h and then heated to 72° C. for 15 min. The DNA was captured on M-270 DynaI beads as per manufacturer's instructions. The beads are then used directly in a labelling reaction using PSAf labelled with Cy5 or Cy3. The following PCR reaction is performed on the beads in a 100 ml volume containing 25 pmol labeled PSAf, 0.2 mM dNTPs and 2.5 U Taq polymerase. The mixture is cycled at 95° C. for 2 min, 93° C. for 15 s, 60° C. for 15 s, 72° C. for 15 s; x 30; 72° C. for 2 min, 4° C. on hold.

### Example 11

### Method for the Production of Probes from Chromatin Fractions for Use in Hybridization to Functional Site Microarrays

Probes were prepared from chromatin fractions according to the following protocol.

A. Formaldehyde Crosslinked Chromatin Fragments were Isolated According to the Following Protocol:

1. Start with nuclei isolated from K562 cells prepared according to the standard tissue preparation protocol. After the nuclei are pelleted they are washed and resuspended in PDS pH 7.4 with 1 mM EDTA and 0.5 mM EGTA and freshly added protease inhibitors.

2. Add formaldehyde to a final concentration of 0.5% and mix gently at room temperature for 10 min.

3. Quench crosslinking reaction by adding 2.5 M glycine to a final concentration of 125 mM. Stir at room temperature for an additional 5 min.

4. Pellet nuclei by spinning for 5 min at 1500 g at 4° and resuspend in the smallest amount of buffer possible. (Having the solution very concentrated here will reduce the need to concentrate it later.

It seems that SDS is not required in this buffer as SDS does not lyse crosslinked cells, but sonication does. One dialysis step will be avoided if the sonication is performed in Xba Digest Buffer (XDB; 10 mM Tris pH 8.0, 1 mM MgCl₂, 50 mM NaCl, 1 mM BME). Maintain conditions as cold as possible.

5. Sonicate to give DNA-protein complexes that have roughly 500 bp of DNA.

B. Digest DNA with XbaI and Exonuclease to Give Single Stranded Regions for Binding of Biotinlyated Primers

1. If the sonication is performed in XDB, immediately add XbaI (10 U/ug DNA) to solution and incubate at 37°. It is preferred to minimize the time at 37°. For example, one can use a 3 hr digestion, adding the enzyme in two different aliquots 1.5 hr apart.

2. λ exonuclease may be added at a final concentration of 1 U/ug DNA directly to the Xba digest and incubated at 370 for 2 h. Quench the reaction with 1 mM EDTA.

C. Capture of Chromatin-Protein Complexes.

This is a two step process. First, biotinylated primers must bind to the HBB HS2 site, and second these biotinylated complexes must bind to Streptavidin-coated Dyna beads.

1. Dialyze into the solution hybridization buffer—perform dialysis at 4°.

a) 10 mM Tris (8.0), 1 mM EDTA, 1 M NaCl,

b) 10 mM Tris (8.0), 1 mM EDTA, 1 M NaCl, 10% DMSO

2. Hybridize with biotinylated primers.

a. Add 6 biotinylated oligos spanning the HBB HS2 site at 3.6 nM each and heat sample to 800 for 10 min. and then cool slowly to 370.

b. Incubate chromatin with biotinylated oligos at 42° C.

3. Capture complexes on Dyna M270 beads.

### Example 12

### Sample Preparation Using Agarose Plugs

Eppendorf tubes were prepared with 0.5 ml 1.4% agarose in 50° C. heating block. The agarose had been prepared in a buffer containing 20 mM Tris.Cl pH 8.0, 75 mM NaCl, and 12 mM EDTA.

DnaseI treated nuclei were prepared as described in Example 2. Following DNaseI treatment, nuclei were resuspended in a buffer containing 1 mM Tris.Cl pH 8.0, 77 mM NaCl, 6 mM KCl, 6 mM CaCl₂, 0.1 mM EDTA, 0.05 mM EGTA, 0.05 mM spermidine, 0.015 mM spermine. EDTA was added to 12 mM (add 50 ul of 250 mM EDTA) in each 1 ml treated nuclei suspension, and the samples were transfered on ice. 0.5 ml of nuclei suspension were mixed with 0.5 ml agarose solution; the samples were mixed well but were not vortexed. Subsequently, the samples were distributed in 75 ul aliquots in plastic molds, allowed to set 5 min at room temperature, then transferred to 4° C. for 15 min. Following this step, the plugs were transferred to microcentrifuge tubes, 2 plugs per 2 ml microcentrifuge tube with 1.0 ml PK buffer (30 mM Tris.Cl, pH 8.0, 100 mM NaCl, 50 mM EDTA, 0.1% SDS, RNAse A 10 ug/ml). The samples were then incubated 15 minutes at 37° C. with no mixing and minimal moving. Proteinase K was then added to 100 ug/ml (from a 19.6 mg/ml stock, 5.1 ul was added to each 1.0 ml). The samples were then incubated an additional 15 min. The buffer was then exchanged for fresh PK buffer (see above), and the samples were incubated an additional 15 min at 37° C. The aforementioned exchange/incubation was repeated once additional time.

The buffer was then removed and the tubes incubated by submersion in 50° C. water bath for 24 hours. Two plugs at a time were then equilibrated in Taq buffer+1 ml PMSF (10 mM Tris.Cl, pH 8.25, 2 mM MgCl2, 50 mM KCl; PMSF 0.2 mM). Two exchanges were performed, with each incubation for 30 min at room temperature. One additional wash without PMSF was also performed.

The plugs were then equilibrated in 1 ml Taq buffer based on 10× stock solution provided with Taq (no PMSF) and left at room temperature for 15 min. The buffer was then replaced with fresh 1×Taq buffer up to a total volume of approx 500 ul. The following reagents were then added:

5 ul dNTPs (10 mM each)

5 ul T4 polymerase

5 ul Taq polymerase

The samples were then incubated for 30 min at 37° C., the first five minutes of which were spent rotating on a horizontal mixer. 5 ul dATP (10 mM) was then added and the samples were mixed by during a further incubation of 5 min while on a horizontal mixer. The samples were then transfer to 55° C. for 30 min. The reaction was then terminated by adding 15 ul 400 mM EDTA (or to 12 mM), with good mixing assured by turning.

DNA was then eluted by use of a Qiagen Qiaexll kit, according to the following protocol:

Add 900 ul Buffer QX1+300 ul H2O (if 4 plugs of 75 ul);

Add 30 ul QIAEX II suspension (vortex 30 sec.);

Incubate at 50° C. 10 min to solubilize agarose and bind DNA;

Mix by vortexing every 2 min;

Colour of the mixture should be yellow;

Centrifuge 30 sec. At 11,000 rcf;

Wash pellet with 500 ul Buffe QX1;

Wash pellet 2× with buffer PE;

Air-dry the pellet 10-15 min.

DNA was eluted by adding 50 ul LOTE (3-0.2) followed by resuspension in the manufacturer-supplied resin. The samples were then incubated for 10 min at 50° C. The samples were then centrifuged for 30 sec. At 11,000 rcf, and the supernatant was pipetted to a clean tube.

### Example 13

### Sample Preparation Using Subtractive Hybridization

Samples were prepared using subtractive methods according to the following protocol.

Driver DNA was prepared in the following way. 50 μl of a solution containing 5 μg of cleaned genomic DNA isolated from nuclei treated with DNaseI was mixed with 36 μl of water, 10 μl of 10× T4 DNA polymerase buffer (NEB), 1 μl of (100 mg/ml) BSA and 1 μl of a solution containing 10 mM dNTPs. This was incubated for 10 minutes at 65° C. for 10 min after which 2 μl of T4 DNA polymerase was added. The mixture was incubated for 15 minutes at 37° C. followed by 15 minutes at 70° C. The sample was then phenol-chloroform extracted and ethanol precipitated, after which it was resuspended in 20 μl water. To this 14 μl of water, 4 μl of 10×NEB Buffer 4, 0.5 μl of BSA and 2 pl of NlaIII (NEB) were added and incubated for 2 hours at 37° C. for 2 hours followed by a 15 minute digestion at 72° C.

To the digested DNA the following reagents were added 7.5 μl of 10 x Exonuclease III buffer (Promega), 23.5 μl of water and 2 μl Exonuclease III (Promega). The mixture was incubated at 25° C. for 3 minutes and then 225 μl Mung Bean Nuclease Master mix (30 μl 10× Mung Bean Nuclease buffer (Promega), 193 μl water, 2 μl Mung Bean Nuclease) was added and the incubation continued for a further 15 minutes. The reaction was stopped by the addition of 30 μl of Stop Buffer (0.3 M Tris-HCl, 50 mM EDTA, pH 8.0) and incubated for a further 3 min. To this 33 μl of 3 M NaOAc pH 7.0 was added and the sample phenol-chloroform extracted and ethanol precipitated. The resultant pellet was resuspended in 17 μl water.

The following oligonucleotides were used to form Linker 1 at a concentration of 250 pmol/μl:

FNMME 5′-CAC GAT CGG CTC GAG TCC GAC CAT G-3′;
RNMME 5′-Phosphate-GTC GGA CTC GAG CCG ATC GTG-3′.

These were ligated to 17 μl sample of restricted DNA by the addition of 59.5 μl of water, 12.5 μl of Linker 1 (250 pmol/μl), 10 μl of 10× T4 DNA ligase (NEB) and 1 μl of High Concentration T4 DNA ligase (400 U). The ligation was incubated overnight at 16° C. and then cleaned on a Qiagen PCR clean up column and eluted in 50 μl volume.

Twenty PCR reactions were assembled in the following way. To 100 ng of ligated Driver DNA the following components were added; 10 μl of 10×Taq buffer+MgCl₂ (Roche), 4 μl of 25 mM MgCl₂, 2 μl of 10 mM (dATP, dCTP, dGTP), 3 μl of 10 mM dUTP, 1.6 μl of FNMME (25 pmol/μl) and water to give a final volume of 99.5 μl and then 0.5 μl Taq polymerase. The PCR reactions were performed with the following cycling parameters: 72° C. for 2 min; 25 cycles of 95° C. for 30 s, 60° C. for 30 s, 72° C. for 2 min; and a final extension time of 72° C. for 5 min.

Tester DNA was prepared in the following way. 2 μg of cleaned genomic in a volume of 20 μl was mixed with 14 of μl water, 4 μl of 10×NEB Buffer 4, 0.5 μl of BSA and 2 μl of NlaIII (NEB). The reaction was incubated at 37° C. for 2 hours.

The following oligonucleotides were used to form Linker 1 at a concentration of 250 pmol/μl:

Biotin-FNMME
5′-Biotin-CAC GAT CGG CTC GAG TCC GAC CAT G-3′
RNMME
5′-Phosphate-GTC GGA CTC GAG CCG ATC GTG-3′

These were ligated to restricted DNA at a molar excess of 50 times more linker. The following components were added to the restricted DNA; 22 μl of water, 5 μl of Biotin-Linker 1 (250 pmol/μl), 5 μl of 10× T4 DNA ligase buffer (NEB) and 1 μl of High Concentration T4 DNA ligase (400 U). The reaction was incubated overnight at 16° C. following which it was cleaned on a Qiagen PCR clean up column and eluted in 50 μl volume. A PCR reaction was performed on 100 ng of the ligated product by the addition of 10 μl of 10× Taq buffer+MgCl₂ (Roche), 2 μl of 10 mM dNTPs, 1.6 μl of a solution of Biotin-FNMME (25 pmol/μl), water added to give a final volume of 99.5 μl and 0.5 μl Taq polymerase. The reaction was performed with the following cycling parameters: 72° C. for 2 min; 25 cycles of 95° C. for 30 s, 60° C. for 30 s, 72° C. for 2 min; and a final extension time of 72° C. for 5 min.

Subtraction was performed with the pool of PCR Driver DNA and the single tube of amplified Tester DNA. These were mixed and 220 μl of 3 M NaOAc pH 5.2 and 2 ml iso-propanol added. The DNA precipitated and resuspended in 100 μl of water and cleaned on a Qiagen PCR column and eluted in 100 μl EB buffer. The sample was precipitated again and resuspended in 6 μl water and placed in a thin walled PCR tube, layered with mineral oil and boiled for 10 minutes. To this 3 μl of Hybridization buffer (1.2 M NaCl, 0.3 M Tris-HCl pH 8.5, 3 mM EDTA) was added. This was incubated for 40 hours at 60° C. After which 195 μl of water was added and the sample phenol chloroform extracted. The aqueous phase was taken and mixed with 26 μl of 10× Uracil DNA glycosylase buffer (Roche) and 30 μl Uracil DNA glycosylase (30 U) and incubated at 37° C. for 4 hours. Following which it was ethanol precipitated and resuspended in 25 μl of TE buffer. To this solution 3 μl of 10× Mung Bean Nuclease (Promega) and 2 μl of Mung Bean nuclease (Promega) was added and the mixture incubated for 30 minutes at 37° C. The reaction was stopped by the addition of 0.6 μl of 50 mM EDTA.

The sample was captured on 10 μl washed M-280 DynaI beads (as instructed by the manufacturer) and the beads resuspended in 20 μl of TE buffer. 0.5 μl of resuspended beads were then mixed with 10 μl of 10×Taq buffer+MgCl₂ (Roche), 2 μl of 10 mM dNTPs, 1.6 μl FNMME (25 pmol/μl) and the volume adjusted to 99.5 μl with water. 0.5 μl Taq polymerase was added and the PCR reaction run on the following program: 72° C. for 2 min; 15 cycles of 95° C. for 30 s, 60° C. for 30 s, 72° C. for 2 min; and a final extension time of 72° C. for 5 minutes.

Up to three more rounds of subtraction of the PCR product with fresh Driver DNA were performed. The PCR product at the end of each subtraction stage represents a Functional Site-enriched population which was used in a labeling reaction according to Example 4.

Alternatively, fractionated DNA was used as a source of Tester DNA. To 250 ng of cleaned fractionated sample 15 μl of 10×PCR buffer+MgCl₂ (Roche), 2 μl of 10 mM dNTPs, 1 μl of Taq polymerase, 1 μl of T4 DNA polymerase and water to give a final volume of 100 μl. The reaction was incubated at 37° C. for 15 minutes followed by 72° C. for 15 minutes and the addition of 1.5 μl of 0.5 M EDTA. The DNA was ethanol precipitated in the presence of 10 μg glycogen and the pellet resuspended in 20 μl of water.

The following oligonucleotides were used to form Linker 1 at a concentration of 250 pmol/μl:

| | |
|---|---|
| B-Sb2F | 5′-Biotin-CTC TGG CGC GCC GTC CTC TCA CGC GTC CGA CT-3′ |
| Sb2R | 5′-Phosphate-GTC GGA CGC GTG AGA GGA CGG CGC GCC AGA G-3′ |

These were ligated to restricted DNA at a molar excess of 50 times more linker. The following components were added to the restricted DNA; 22 μl of water, 5 μl of Biotin-Linker 1 (250 pmol/μl), 5 μl of 10× T4 DNA ligase buffer (NEB) and 1 μl of High Concentration T4 DNA ligase (400 U). The reaction was incubated overnight at 16° C. following which it was cleaned on a Qiagen PCR clean up column and eluted in 50 μl volume. To this sample 19.5 μl of water, 8 μl of 10×NEB Buffer 4, 0.5 μl of BSA and 2 μl of NlaIII (NEB) was added and the mixture incubated for 2 hours at 37° C. followed by 72° C. for 15 minutes.

The following oligonucleotides were used to form Linker 1 at a concentration of 250 pmol/μl:

Sb3F 5′-CAC GAT CGG CTC GAG TGA GAC CAT G-3′
Sb3R 5′-Phosphate-GTC TCA CTC GAG CCG ATC GTG-3′

These were ligated to restricted DNA at a molar excess of 50 times more linker. The following components were added to the restricted DNA; 8 μl of water, 1 μl of Biotin-Linker 1 (250 pmol/μl), 10 μl of 10× T4 DNA ligase buffer (NEB) and 1 μl of High Concentration T4 DNA ligase (400 U). The reaction was incubated overnight at 16° C. following which it was cleaned on a Qiagen PCR clean up column and eluted in 50 μl volume.

To 25 μl of the sample 10 μl of 10×Taq buffer+MgCl₂ (Roche), 2 μl of 10 mM dNTPs, 1.6 μl of Biotin-Sb2F (25 pmol/μl), 0.5 μl of Taq polymerase, 1.6 μl Sb3F (25 pmol/μl) and water to a final volume of 99.5 μl were added. The PCR reaction was run on the following program: 72° C. for 2 min; 25 cycles of 95° C. for 30 s, 60° C. for 30 s, 72° C. for 2 min; and a final extension time of 72° C. for 5 minutes. This tester DNA was subtracted from Driver DNA, prepared as described above, in a similar fashion as stated, with the exception that the final PCR contained the following primers: 1.6 μl of Sb2F (25 pmol/μl) and 1.6 μl of Sb3F (25 pmol/μl). The PCR product at the end of each subtraction stage again represents a Functional Site-enriched population which was used in a labeling reaction according to Example 4.

### Example 14

### Preparation and Labeling of Control DNA Fragments for Array Hybridization

Genomic DNA was isolated from K562 nuclei which had not been treated with a nuclease (1 ml of nuclei with an A₂₆₀ of 8 OD/ml) and had been subsequently digested with NlaIII to completion or sonicated to give fragments of a certain average length and the DNA purified using a Qiagen Dneasy column. The concentration of the DNA was corrected to 150 ng/μl. These probes were labeled with Cy3 or Cy5 according to the protocol of Example 4.

### Example 15

### Chromatin Fractionation by Ultracentrifugation in Sucrose Gradients

In a first experiment, 10⁷ nuclei were digested with DNaseI and stop the reaction by addition of EDTA from a 0.1 M stock to a final concentration of 10 mM and chill on ice. The nuclei were lysed by dialysis into 0.2 mM EDTA, pH 7.0 overnight at 4° C. in a volume of 1 ml.

The lysed nuclei were layered onto a 15.5 ml 5-30% continuous sucrose gradient (prepared in 10 mM triethanolamine.HCl, 1 mM EDTA, 0.5 mM PMSF, pH 7.0) and spun in an SW28 rotor overnight (16 h) at 28000 rpm.

The gradients were fractionated and the size of DNA fragments determined by agarose gel electrophoresis. Typically, those fractions of subnucleosomal size (<150 bp) were labeled for use as probes by random priming.

In a second experiment, linear sucrose gradients were formed using 10% and 40% sucrose solutions prepared in 20 mM Tris.Cl pH 7.4, 1 M NaCl, 1 mM EDTA. Before loading of DNA samples, they were incubated for 65° C. for 5 minutes. The gradients were then centrifuged at 30,000 rpm, at 20° C. for 24 hours. The result of this process is illustrated in FIG. 11. Following this, they were fractionated by removal of successive 0.75 ml fractions from the top and the DNA precipitated using isopropanol, 0.3 M NaOAc and Novagen (a co-precipitating agent).

### Example 16

### Chromatin Solubility Fractionation

DNaseI digestion of nuclei was performed as described in Example 2. The reactions were stopped by the addition of 10 mM EDTA and the nuclei pelleted by centrifugation at 2, 000 g for 5 minutes before being resuspended in a buffer containing 0.2 mM EDTA, 0.5 mM DTT, 0.5 mM PMSF and incubated on ice for 2 hours.

The material was then centrifuged at 3, 000 g for 5 minutes and the supernatant loaded onto sucrose gradients for fractionation by ultracentrifugation, essentially as described above in Example 15, except they were run on 5-30% linear sucrose gradients spun at 30, 000 rpm for 18 hours. Fractions were treated with 50 μg/ml RNase by incubation for 30 minutes at 37° C., after which EDTA was added to a final concentration of 5 mM and SDS to 0.5% (v/v) and Proteinase K added to a final concentration of 50 μg/ml. The fractions were incubated overnight at 56° C. before phenol-chloroform extraction and ethanol precipitation in the presence of a DNA carrier (10 μg/ml glycogen).

### Example 17

### Ligation of Linker to Repaired Dnase I Cut Sites

The primers F-Bsg (5′-Biotin-TEG-tct gca cga tca age acg tgc ag-3′) and R-Bsg (5′-ctg cac gtg ctt gat cgt gca ga-3′) were resuspended in a 100 μl solution of 50 MM NaCl at concentrations of 100 pmol/μl and the mixture heated to 95° C. for 2 minutes then slowly allowed to cool to room temperature.

20 μg of genomic DNA from a DNaseI-treated nuclei was repaired with T4 DNA polymerase in a 100 μl reaction volume containing 50 U T4 DNA polymerase (Promega) in the manufacurer's recommended buffer supplemented with 0.2 mM dNTPs and 0.1 mg/ml BSA (Bovine Serum Albumin).

The mixture was incubated at 37° C. for 10 min before the enzyme was heat inactivated at 75° C. for 15 min and the DNA was cleaned, typically by use of a Qiagen Dneasy column and digested overnight to completion with NlaIII (New England Biolabs) as per the manufacturer's instructions.

The DNA was recovered following extraction with phenol-chloroform, chloroform and ethanol precipitation in the presence of 0.3 M NaOAc. The washed pellet was resuspended in 40 μd water. 1 nmole of the Bsg adapter was ligated on to this DNA sample in a final reaction-volume of 50 μd in the presence of T4 DNA ligase (Promega) by incubation overnight at 4° C. The ligation products were captured by mixing with Paramagnetic beads (DynaI) for 60 min at 37° C. with occasional agitation. The beads were separated on a magnetic stand and washed several times in the recommended buffer (10 mM Tris.HCl, 1 M NaCl, 1 mM EDTA, pH 8.0) and finally resuspended in 50 μl of 10 mM Tris.HCl, pH 8.0.

### Example 18

### Ligation of Linker to A-Tailed Dnase I Cut Sites

Linkers were ligated to A-tailed DnaseI cut sites according to the following protocol:

Wash 20 μg gDNA on a Centricon 30 column (as instructed per manufacturers) and elute with 200 μl TE pH 8.0 following centrifugation at 6 000 rcf for 3 mins.

To 100 μl cleaned gDNA mix 11 μl 10×PCR buffer supplemented with MgCl₂ (Roche) and incubate at 65° C. for 10 mins. then place on ice whilst the following tailing mix is added:

4 μl 1 Ox PCR buffer supplemented with MgCl₂;

2 μd 10 mM dNTPs;

1 μl T4 DNA polymerase (5 U/μl; Roche);

1 μl Taq polymerase (3 U/μl; Roche);

30 μl water.

Incubate at 37° C. for 15 mins followed by 15 mins at 72° C. then clean on Qiagen-PCR Clean-up column and elute in 150 μl EB.

A linker is prepared from the following oligonucleotides:

PS_0016_F 5′Biotin-
CTC TGG CGC GCC GTC CTC TCA CGC GTC CGA CT
PS_0016_R
GAG ACC GCG CGG CAG GAG AGT GCG CAG GCT G - 5′
Phos.

To 143 μl repaired DNA add the following:

16 μl 10× T4 DNA ligase buffer (NEB);

1 μl Linker (50 pmol/μl);

0.5 μl High concentration T4 DNA ligase (NEB; 400 U/μl).

Clean ligation using Qiagen PCR column and elute with 500 pd TE buffer preheated to 55° C.

### Example 19

### Ligation of Secondary Linker to Restriction Site Proximal to Dnase I Cut Site

Secondary linkers were ligated to restriction sites proximal to Dnasel cut sites according to the following protocol:

A. Blunt with T4 DNA Polymerase.

Mix:

50.0 μl DNA

36.0 μl H₂O

10.00 μl 10× T4 DNA polymerase buffer (NEB)

1.00 μl BSA

1.00 μl 10 mM dNTPs

2.00 μl T4 DNA polymerase

37° C./15 min.

70° C./15 min.

B. dA Tailing with Taq.

Add 0.50 μl Taq Polymerase

72° C./10 min.

Clean up DNA w/ Qiagen PCR kit.

Elute DNA in 50.0 μl Elution Buffer (10 mM Tris.Cl pH 8.0)

C. Adaptor Ligation (PS003F/R)

1. Resuspend oligos at 1 mM in 10 mM Tris (pH 8.0)

2. Anneal Oligos:

Mix:

5.00 pd 2× annealing buffer (100 mM NaCl, 20 mM Tris-HCL (pH 8.0),

2 mM EDTA=2×Binding Buffer).

3.00 μl H₂O

1.00 pl PS0003F (MWG; 1 mM)

1.00 pl PS0003R (MWG; 1 MM)

Heat to 80° C., cool to 25° C. over 1 Hr.

Adaptor Concentration=100 pmole/μl=100 pM

3. Phosphorylate Adaptor.

Mix:

10.00 μl Adaptors

5.00 μl 10× Ligase buffer

1.00 μl PNK (NEB; 5U/μl)

34.0 μl H2O

37° C./30 min

Adaptor Concentration=20 pmole/μl=20 μM

4. Adaptor Ligation:

Mix:

37.5 μl H₂O

50.0 μl dA tailed DNA

10.00 μl Ox Ligase buffer

2.50 μl PS003F/R+PNK Adaptor (50 pmol)

4° C./16 Hrs.

65° C./20 min.

Add 10.0 μl 3M NaOAc, ppt. W/200.0 μl EtOH

Wash 70% EtOH

Resuspend in 20.0 μl 0.5×TE

Remove 0.5 μl and add to 9.5 μl TE for QC gel.

D. Hsp92 II Digest

Mix:

19.50 μl DNA

23.5 μl H2O

5.00 μl 10×Buf. K (Promega)

0.50 μl BSA (Promega)

2.00 μl Hsp92 II (Promega; 10 U/μl)

37° C./2 Hrs

Add another 2.00 μl Hsp92 II

37° C./1 Hrs

Remove 1.00 μl and add to 9.00 μl TE for QC gel

Remove 2.00 μl and add to 98.0 μl and measure AZeo

Heat remaining sample 72° C./15 min.

E. Capture DNA with Dynabeads

1. Wash M270 Dynabeads.

50.0 μl Dynabeads

wash 2×200 μl 1× Binding Buffer (10 mM Tris, 1 mM EDTA, 1 M NaCl; pH 8.0)

Resuspend Beads in 50 μl 1×BB

2. Prepare DNA

Add 50.0 μl 2×BB to DNA, mix well.

3. Bind DNA to Dynabeads

Mix DNA and washed Dynabeads.

37° C./1 Hrs w/ occasional mixing.

Capture beads-retain S/N=SN1

Wash beads 2×200 μl TE

Wash beads 1×200 μl 1× Ligase buffer.

Note: Could take an aliquot of beads for direct cloning: proceed to Not I digest.

F. Second Adaptor Ligation (HspF/R)

Resuspend Beads in 100 μl Ligation Mater Mix:

85.5 μl H₂O

10.00 μl 10× Ligase Buffer

2.50 μl HspF/R+PNK Adaptors (50 pmole)

2.00 μl T4 DNA Ligase

16° C./16 Hrs.

65° C./20 min.

Capture beads

Wash 2×200 μI TE

Wash 1×200 μl 1×NEB3 buffer

G. Not I Digest

Resuspend Beads in 100 μl Not I Master Mix:

85.0 μl H2O

10.00 μl 10×NEB3 buffer

1.00 μl BSA

4.00 μl Not I (NEB, 10U/VI)

37° C./1 Hrs w/ occasional mixing.

Capture beads, retain S/N=SN2

Wash beads 1×100 μl TE, retain S/N and pool with SN2.

Add 20.0 μl 3M NaOAc to SN2

Add 1.00 μl Glycogen

Ppt. W/ 440 μl EtOH

Wash DNA 70% EtOH.

Resuspend DNA in 10.0 μl 10 mM Tris (pH 8.0)

### Example 20

### Biotinylation of DnaseI Ends with Terminal Transferase and Biotin-DDNTP

The ends of DNA fragments generated by DNase I digestion were biotinylated using terminal transferase and biotin-ddNTP according to the following protocol:

A 10 μl solution containing 10 μg of cleaned and T4 DNA polymerase-repaired DNaseI treated genomic DNA was incubated with:

4 μl 5× Terminal transferrase buffer (Roche);

4 μl 25 MM COCl₂;

1 μl 1 mM biotin-ddUTP;

1 μl Terminal transferase (15 U/μl; Roche);

10 μl water.

The mixture was Incubated at 37° C. for 15 mins. The reaction was then cleaned up on Qiagen DNEasy column as per manufacturer's instructions, eluted in 200 μl of EB, and captured on DynaI beads as per manufacturer's instructions.

### Example 21

### Embedding Dnase I-Digested Nuclei in Agarose Plugs

10⁷ K562 nuclei were treated with various amounts of DNaseI for 3 mins at 37° C. in the presence of a buffer containing 6 mM CaCl₂. The reactions are stopped by mixing with an equal volume of pre-melted 1% low melting point agarose cast in 20 mM Tris.Cl, 20 mM EDTA, 10 mM EGTA, pH 8.0 stored ata temperature of 50° C. The solutions are mixed by gentle inversion, 100 μI moulds poured and allowed to set in the fridge.

Subsequently the gel plugs are incubated in 5 ml Proteinase K buffer (1% SDS, 0.5 M EDTA pH 9.), 100 μl/ml Proteinase K) at 50° C. for 24 hours (with no shaking).

The following morning the buffer was changed by washing the plugs three times for one hour with the different buffer. The high molecular weight genomic DNA captured in the agarose plugs was treated as soluble genomic DNA was previously.

### Example 22

### TSC-Ligation Mediated PCR Amplification of Array Probes

TSC-ligation mediated PCT amplification of array probes was performed according to the following protocol:

Wash 20 μg gDNA on a Centricon 30 column (as instructed per manufacturers) and elute with 200 μl TE pH 8.0 following centrifugation at 6 000 rcf for 3 mins.

To 100 μl cleaned gDNA, mix 11 μl 10×PCR buffer supplemented with MgCl₂ (Roche) and incubate at 65° C. for 10 mins. then place on ice whilst the following tailing mix is added:

4 μl 1 Ox PCR buffer supplemented with MgCl₂;

2 μl 10 mM dNTPs;

1 μl T4 DNA polymerase (5 U/μl; Roche);

1 μl Taq polymerase (3 U/μl; Roche);

30 μl water.

Incubate at 37° C. for 15 mins followed by 15 mins at 72° C. then clean on Qiagen PCR Clean-up column and elute in 150 μl EB.

A linker is prepared from the following oligonucleotides:

PS_0016_F 5′Biotin-
CTC TGG CGC GCC GTC CTC TCA CGC GTC CGA CT
PS_0016_R
GAG ACC GCG CGG CAG GAG AGT GCG CAG GCT G - 5′
Phos.

To 143 μl repaired DNA add the following:

16μ 10× T4 DNA ligase buffer (NEB);

1 μl Linker (50 pmol/μl);

0.5 μl High concentration T4 DNA ligase (NEB; 400 U/μl).

Clean ligation using Qiagen PCR column and elute with 50 μl EB buffer preheated to 55° C. Add the following components:

20 μl of 10×NEB buffer 4;

2 μl 100×BSA;

3 μl NlaIII (10 U/μl; NEB);

145 μl water.

Incubate overnight at 37° C. and then heat inactivate the enzyme by incubation at 75° C. for 15 mins.

Wash 20 μl DynaI beads M-270 (DynaI, Norway) in two changes of 200 μl of 1× Wash buffer (10 mM Tr1S.HC1, 1 M NaCl, 1 mM EDTA, pH 8.0), capture beads on magnetic stand and remove supernatant. Resuspend beads in 200 μl 2×Wash buffer and mix by gentle pipetting with 200 μl of digested genomic DNA. Incubate for 1 h at 37° C. after which the beads are recaptured and washed again in two changes of 1× Wash buffer. The captured beads are then resuspended gently by addition of the following mixture:

4 μl 10×NEB buffer 4;

0.4 μl 100×BSA;

34.6 μl water;

1 μl MmeI (NEB; 10 U/μl).

Incubate for 2 h at 37° C. Capture on DynaI beads and wash twice in 1×Wash buffer, then resuspend beads in 8 μl 0.1 M NaOH and incubate with gentle incubation at room temperature for 5 min.

Capture beads and resuspend by addition of the following reagents:

2.5 μl Tsc Incubation buffer (Roche);

+1.2 μl NotAd (10 pmol/μl; 5′Phopshate-TAT GCG GCC GCT TAG TAC-3′);

+1.2 μl 3J (10 pmol/μl; 5′-NNN NAT ATG CGC-3′);

+1 μl Tsc ligase (Roche);

+19.1 μl water.

Incubated using the following programme: 94° C. for 5 min; 94° C. for 30 s followed by 30° C. for 3 min; this step repeated 32 times; 99° C. for 15 min; 4° C. for ever.

1 μl of the Tsc ligation products can then be amplified in the following PCR reaction to produce a labeled product:

10 μl 10×Taq polymerase buffer supplemented with MgCl₂ (Roche);

1 μl 25 pmol/μl Cy5-labeled PS_{—}0016_F;

1 μl 25 pmol/μl NotAdR (5′-GTA CTA AGC GGC CGC ATA-3′);

2 μl 10 mM dNTPs;

84.5 μl water

0.5 μl Hot-start Taq polymerase (3 U/μl; Roche).

The reaction ran on the following program: 95° C. for 5 mins; 93° C. for 15 s, 60° C. for 15 s, 72° C. for 20 s x 30 cycles; 72° C. for 60 s, 4° C. on hold. The PCR products were then cleaned on a Qiagen PCR clean up column (as per the manufacturer's instructions) and used as a probe.

### Example 23

### TSC-BST Amplification of Array Probes

Array probes were prepared according to the following protocol:

Biotinylating DNaseI Cut Sites

Treat 10 μl (1 Ixg) of genomic DNA which either has (+) or has not (−) been treated with DNaseI with T4 DNA polymerase by assembling the following reaction:

4 μl Roche 5× Terminal transferase buffer;

4 μl 25 MM COCl₂;

1 μl Terminal transferase (Roche, 50 U/μl);

1 μl mM ddUTP-Biotin (Roche).

Incubate at 37° C. for 30 mins

Clean up on Qiagen Dneasy by adding 20 μl Proteinase K, 200 μl AL

Vortex heat at 65° C. for 15 min

Add 200 μl Ethanol mix well and spin through column for 1 min

Wash 500 μl AW 1 followed by 500 μl AW2

Elute with 150 μl AE buffer

Digestion with DNaseI to produce random fragments with a size of 500 bp

To the cleaned DNA add the following components:

20 μl 10× DNase I buffer (67 mM Tris.HCl, 0.67 M NaCl, 67 mM MnCl₂, pH 7.5);

1 μl DNase I (0.1 U/μl);

29 μl water.

Incubate at room temperature for 15 mins

Reaction was stopped by the addition of 200 μl phenol:chloroform and extracted

Extracted with chloroform and ethanol precipitated

Material resuspended in 200 μl of 1× Binding Buffer and captured on 20 μl prewashed

DynaI beads

Beads were washed twice in 200 μl of 1× Binding Buffer

Isolation of Supernatant and TSC Ligase Treatment

DynaI beads are captured on magnetic strand and incubated in 50 μl of 0.15 M NaOH at room temperature for 10 mins.

DynaI beads are captured and the supernatant carefixlly removed and mixed with 50 μl 0.15 M HCl, 11 μl 100 mM Tris.HCl pH 8.0

1 μl 10 mg/ml glycogen is added and the DNA precipitated in the presence of 0.3 M NaOAc pH 5.2 and 0.6 volumes isopropanol

Have synthesized the following primers: NotAd (5′-Phopshate-TAT GCG GCC GCT TAG TAC-3′); 3′J (5′-CCG CAT ANN NN-3′); 5′J (5′-NNN NGT ACT MG G-3′); NotAdR (5′-GTA CTA AGC GGC CGC ATA-3′). Redissolve the DNA/glycogen pellet in 10 μl water and assemble the following reaction:

1 μl 1 pmol/μl NotAd;

1 μl 1 pmol/μl 3′J;

1 μl 1 pmol/μl 5′J;

2.5 μl 10×Tsc Ligase buffer (Roche, pre-aliquoted);

1 μl Tsc Ligase (5 U/μl); 9.5 μl water.

Incubate in a Thermal-cycler with the following program: 94° C. for 30 s; 94° C. for 15 s, 40° C. for 3 mins, x 32; 99° C. for 10 mins.

Digestion with Exonuclease I (Isolation of ccDNA)

To 20 μl of the Tsc reaction add the following components:

2.5 μl Roche 10× Exonuclease I buffer;

1 μl Exonuclease 1(10 u/μl);

1 μl 10 mM dNTP mix (Roche);

1.5 μl water.

Incubate at 37° C. for 2 h

Precipitate the DNA by the addition of the following reagents:

1 μl 10 mg/ml glycogen;

2.5 μl 3M NaOAc pH 5.2;

55 μl Absolute ethanol.

Precipitate, wash and resuspend in 20 μl water.

Bst polymerase mediated Rolling Circle Amplification (RCA)

15 μl of resuspended ccDNA was amplified using Bst polymerase (NEB) in the following reaction:

5 μl 10×Bst polymerase buffer;

3 μl 100 pmol/μl NotAdR;

1 μl 10 mM 5:1 dNTPs;

3 μl 1 mM Cy5-dCTP;

22 μl water.

Incubate at 95° C. for 1 min then cool to 60° C. and add 1 μl Bst polymerase (U/μl) and continue to incubate for 20 h

Release of Monomers by Not I Digestion

1 μl of RCA DNA is digested with Not I in the following reaction:

2 μl 10×NEB Buffer 3;

1 41 Not I (10 U/ml);

16 μl water.

Incubated for 2 h (or overnight if it proves resistant) at 37° C.

Clean on Qiagen PCR purification kit.

### Example 24

### Creation of Indirect Genomic Tags Following Biotinylation of DNASE I Cleavage Site

Indirect genomic tags were generated according to the following protocol:

A 10 μl solution containing 10 ug of cleaned and T4 DNA polymerase-repaired DNaseI treated genomic DNA is incubated with:

4 μl 5× Terminal transferrase buffer (Roche);

4 μl 25 mM COClz;

1 μl 1 mM biotin-ddUTP;

1 μl Terminal transferase (15 U/μl; Roche);

10 μl water.

Incubate at 37° C. for 15 mins then clean up reaction on Qiagen DNEasy column as per manufacturer's instructions. Elute in 30 μl of EB and digest to completion with NlaIII by the addition of:

20 μl of 10×NEB buffer 4;

2 μl 100×BSA;

3 μl NlaIII (10U/μl; NEB);

145 μl water.

Incubate overnight at 37° C. and then heat inactivate the enzyme by incubation at 75° C. for 15 mins.

A linker is prepared from the following oligonucleotides:

| | |
|---|---|
| CO1_F | 5′-ATC CGA TCC GCA TGC GTG CAG CAT G |
| COI UR | TAG GCT AGG CGT ACG CAC GTC - 5′ Phos. |

Wash 20 μl DynaI beads M-270 (DynaI, Norway) in two changes of 200 μl of 1× Wash buffer (10 mM Tris.HCl, 1 M NaCl, 1 mM EDTA, pH 8.0), capture beads on magnetic stand and remove supernatant. Resuspend beads in 200 μl 2× Wash buffer and mix by gentle pipetting with 200 μl of digested genomic DNA. Incubate for 1 h at 37° C. afterwhich the beads are recaptured and washed again in two changes of 1× Wash buffer. The captured beads are then resuspended gently by addition of the following mixture:

4 μl 10× T4 DNA ligase buffer (NEB);

1 μl Linker COI (50 pmol/μl);

34.5 μl water;

0.5 μl High concentration T4 DNA ligase (NEB; 400 U/μl).

Incubate overnight at 16° C. Afterwhich the beads are captured and the unicorporated linker removed by successive washes in 200 μl 1× Wash buffer. The captured beads are then resuspended gently by addition of the following mixture:

4 μl 10×NEB buffer 4;

0.4 μl 100×BSA;

1 μl BsgI (10 U/μl; NEB)—a type 11 s restriction enzyme;

34.6 μl water.

Incubate for 2 h at 37° C. Afterwhich the beads are captured and the supernatant retained. The DNA is precipitated following addition of 1 μl 10 mg/ml glycogen and phenol/chloroform extraction. The DNA pellet is resuspended in 20 μl water.

A linker is prepared from the following oligonucleotides:

| | |
|---|---|
| CO2_F | 5′-GGC AGC CAT GAC CAT CGG CAT GCN N |
| CO2_R | CCC TCG GTC CTG CTA GCC GTA CG-5′ Phos. |

The following ligation is set up by adding the following components to the 20 μl DNA solution:

14 μl 10× T4 DNA ligase buffer (NEB);

1 pl Linker CO 2 (50 pmol/μl);

14.5 μl water;

0.5 μl High concentration T4 DNA ligase (NEB; 400 U/μl).

Incubate overnight at 16° C. Store at −20° C.

To 1 μl of ligation product assemble the following PCR reaction:

10 μl 10×Taq polymerase buffer supplemented with MgCl₂ (Roche);

1 μl 25 pmol/μl Cy5-labeled COIF;

1 μl 25 pmol/μl CO2 F;

2 μl 10 mM dNTPs;

84.5 μl water

0.5 μl Hot-start Taq polymerase (3 U/μl; Roche).

The reaction ran on the following program: 95° C. for 5 mins; 93° C. for 15 s, 60° C. for 15 s, 72° C. for 20 s×30 cycles; 72° C. for 60 s, 4° C. on hold. The PCR products are then cleaned on a Qiagen PCR clean up column (as per the manufacturer's instructions) and used as a probe.

### Example 25

### Creation of Indirect Genomic Tags Following A-Tailing of DNASEI cut Site

Indirect genomic tags were prepared according to the following protocol:

Wash 20 μl gDNA on a Centricon 30 column (as instructed per manufacturers) and elute with 200 μl TE pH 8.0 following centrifugation at 6 000 ref for 3 mins.

To 100 μl cleaned gDNA mix 11 μl 10×PCR buffer supplemented with MgCl₂ (Roche) and incubate at 65° C. for 10 mins. then place on ice whilst the following tailing mix is added:

4 μl 10×PCR buffer supplemented with MgCl₂;

2 μl 10 mM dNTPs;

1 41 T4 DNA polymerase (5U/41; Roche);

141 Taq polymerase (3 U/μl; Roche);

30 μl water.

Incubate at 37° C. for 15 mins followed by 15 mins at 72° C. then clean on Qiagen PCR Clean-up column and elute in 150 μl EB.

A linker is prepared from the following oligonucleotides:

| | |
|---|---|
| PS_0016_F | 5′Biotin-CTC TGG CGC GCC GTC CTC TCA CGC GTC CGA CT |
| PS_0016_R | GAG ACC GCG CGG CAG GAG AGT GCG CAG GCT G-5′ Phos. |

To 143 μl repaired DNA add the following:

16 μl 10× T4 DNA ligase buffer (NEB);

1 μl Linker (50 pmo1/μl);

0.5 μl High concentration T4 DNA ligase (NEB; 400 U/μl).

Clean ligation using Qiagen PCR column and elute with 50 μl EB buffer preheated to 55° C. Add the following components:

20 μl of 10×NEB buffer 4;

2 μl 100×BSA;

3 μl NlaIII (10 U/μl; NEB);

145 μl water.

Incubate overnight at 37° C. and then heat inactivate the enzyme by incubation at 75° C. for 15 mins.

A linker is prepared from the following oligonucleotides:

| | |
|---|---|
| CO1_F | 5′ -ATC CGA TCC GCA TGC GTG CAG CAT G |
| CO1_R | TAG GCT AGG CGT ACG CAC GTC-5′ Phos |

Wash 20 μl DynaI beads M-270 (DynaI, Norway) in two changes of 200 μl of 1× Wash buffer (10 mM Tris.HCl, 1 M NaCl, 1 mM EDTA, pH 8.0), capture beads on magnetic stand and remove supernatant. Resuspend beads in 200 μl 2× Wash buffer and mix with digestion reaction by gentle pipetting. Incubate for 1 h at 37° C. Capture beads on a magnetic stand and resuspend beads in the following reagents:

4 μl 10× T4 DNA ligase buffer (NEB);

1 μl Linker CO1 (50 pmo1/μl);

34.5 μl water;

0.5 μl High concentration T4 DNA ligase (NEB; 400 U/μl).

Incubate overnight at 16° C. Store at-20° C.

To 1 μl of ligation product assemble the following PCR reaction:

10 μl 10×Taq polymerase buffer supplemented with MgCl₂ (Roche);

1 μl 25 pmol/μl Cy5-labeled CO1 F;

1 μl 25 pmol/μl PS_{—}0016_F;

2 μl 10 mM dNTPs;

84.5 μl water

0.5 μl Hot-start Taq polymerase (3 U/μl; Roche).

The reaction ran on the following program: 95° C. for 5 mins; 93° C. for 15 s, 60° C. for 15 s, 72° C. for 20 s×30 cycles; 72° C. for 60 s, 4° C. on hold. The PCR products are then cleaned on a Qiagen PCR clean up column (as per the manufacturer's instructions) and used as a probe.

### Example 26

### Subtraction Off A Functional Site Enriched Sample from a Functional Site-Depleted Sample

A functional site enriched sample was subtracted from a functional site depleted sample by generating tester and driver populations and performing subtractive hybridization as described in the following protocol:

I. Tester Population

A. Blunt with T4 DNA polymerase.

Mix:

50.0 μl DNA

36.0 μl H₂O

10.00 pi 10× T4 DNA polymerase buffer (NEB)

1.00 μl BSA

1.00 μl 10 mM dNTPs

2.00 μl T4 DNA polymerase

37° C./15 min.

70° C./15 min.

B. dA Tailing with Taq.

Add 0.50 μl Taq Polymerase

72° C./10 min.

Clean up DNA w/ Qiagen PCR kit.

Elute DNA in 50.0 μl Elution Buffer (10 mM Tris)

C. Adaptor Ligation (PS003F/R)

1. Resuspend oligos at 1 mM in 10 mM Tris (pH 8.0)

2. Anneal Oligos:

Mix:

5.00 μl 2× annealing buffer (100 mM NaCl, 20 mM Tris-HCL (pH 8.0),

2 mM EDTA=2× Binding Buffer).

3.00 μl H₂O

1.00 μl PS0003F (MWG; 1 mM)

1.00 μl PS0003R (MWG; 1 mM)

Heat to 80° C., cool to 25° C. over 1 Hr.

Adaptor Concentration=100 pmole/μl=100 pM

3. Phosphorylate Adaptor.

Mix:

10.00 μl Adaptors

5.00 μl 10× Ligase buffer

1.00 μl PINK (NEB; U/μl)

34.0 μl H₂O

37° C./30 min

Adaptor Concentration=20 pmole/l=20 μM

4. Adaptor Ligation:

Mix:

37.5 μl H2O

50.0 μl dA tailed DNA

110.00 μl 10× Ligase buffer

2.50 μl PS003F/R+PNK Adaptor (50 pmol)

4° C./16 Hrs.

65° C./20 min.

Add 10.0 μl 3M NaOAc, ppt. W/ 200.0 μl EtOH

Wash 70% EtOH

Resuspend in 20.0 μl 0.5×TE

Remove 0.5 μl and add to 9.5 μl TE for QC gel.

D. Hsp92 II Digest

Mix:

19.50 μl DNA

23.5 Vi H₂O

5.00 μl 10×Buf. K (Promega)

0.50 μl BSA (Promega)

2.00 μl Hsp92 II (Promega; 10 U/μl)

37° C./2 Hrs

Add another 2.00 μl Hsp92 II

37° C. 11 Hrs

Remove 1.00 μl and add to 9.00 μl TE for QC gel

Remove 2.00 μl and add to 98.0 μl and measure A₂₆₀

Heat remaining sample 72° C./15 min.

E. Capture DNA with Dynabeads

5. Wash M270 Dynabeads.

50.0 μl Dynabeads

wash 2×200 μl 1× Binding Buffer (10 μM Tris, 1 mM EDTA, 1 MNaCl; pH 8.0)

Resuspend Beads in 50 μl 1×BB

6. Prepare DNA

Add 50.0 μl 2×BB to DNA, mix well.

7. Bind DNA to Dynabeads

Mix DNA and washed Dynabeads.

37° C./I Hrs w/ occasional mixing.

Capture beads—retain S/N=SN1

Wash beads 2×200 μl TE

Wash beads 1×200 μl 1× Ligase buffer.

Note: Could take an aliquot of beads for direct cloning: proceed to Not I digest.

F. Second Adaptor Ligation (HspF/R)

Resuspend Beads in 100 μl Ligation Mater Mix:

85.5 μl H₂O

10.00 μμl 10× Ligase Buffer

2.50 μl HspF/R+PNK Adaptors (50 pmole)

2.00 μl T4 DNA Ligase

6° C./16 Hrs.

65° C./20 min.

Capture beads

Wash 2×200 μl TE

Wash 1×200 μl lx NEB3 buffer

G. Not I Digest

Resuspend Beads in 100 μl Not I Master Mix:

85.0 μl H2O

10.00 μl 10×NEB3 buffer

1.00 μl BSA

4.00 μl Not I (NEB, 10U/μl)

37° C./1 Hrs w/ occasional mixing.

Capture beads, retain S/N=SN2

Wash beads 1×100 μl TE, retain S/N and pool with SN2.

Add 20.0 μl 3M NaOAc to SN2

Add 1.00 μl Glycogen

Ppt. W/ 440 μl EtOH

Wash DNA 70% EtOH.

Resuspend DNA in 10.0 μl 10 mM Tris (pH 8.0)

II. Driver Population

A. Setup Restriction Enzyme digests.

1. Pst I

20.00 VI DNA

5.00 μl 10×NEB3

24.0 μl H2O

1.00 al Pst I (NEB 20 U/μl)

2. Sph I

20.00 al DNA

5.00 μl 10×NEB2

21.0 μl H2O

4.00 μl Sph I (NEB 5 U/μl)

3. Nsi I

20.00 μl DNA

5.00 μl 10× Nsi buffer

23.0 μl H₂O

2.00 μl Nsi I (NEB 10 U/μl)

4. Sac I

20.00 μl DNA

5.00 μl 10×NEB I

1.00 μl BSA

23.0 μl H₂O

1.00 μl Sac I (NEB 20 U/μl)

Mix well, 37° C./1 Hrs

65° C./20 min.

Add 50.0 μl H₂O+10.00 μM NaOAc

Phenol extract

Ppt. W/ 220 μl EtOH

Resuspend DNA in 10.00 μl 10 mM Tris

Remove 1.00 μl and add to 99.0 μl H₂O and measure A₂₆₀

B. Nuclease Treatment.

Mix:

10.0 μl Digested DNA

7.50 μl 10×ExoIII buffer

H₂O to 73.0 μl

2.00 μl ExoII nuclease

25° C./3 min.

Add 225 μl Mung bean Nuclease Master Mix:

30.00 μl 10×Mung Bean buffer

193.0 μl H₂O

2.00 μl Mung Bean Nuclease

25° C./15 min.

Add 30.0 25° C./3 min. Stop buffer (300 mM Tris (pH 8.0), 50 mM EDTA)

Add 33.0 μl 3 M NaOAC

Phenol extract

Ppt. w/ 660 μl EtOH

Resuspend DNA in 22.0 μl 10 mM Tris.

C. Terminal Transferase.

Mix:

22.0 μl DNA

8.00 μl 10× TdT buffer (Roche)

8.00 μl CoCl2 (Roche, 25 mM)

1.00 μl ddUTP-Biotin (Roche; 1 mM)

1.00 μl TdT (Roche, 25 U/μl)

37° C./15 min.

Ppt w/:

4.00 μl 0.2 M EDTA

5.00 μl LiCl

150 μl EtOH

Resuspend DNA in 10.0 μl H₂O

D. Photo Biotin.

Mix:

10.0 μl DNA

10.00 μl Photo biotin

Place on ice and expose to sun lamp 15 min.

Add 30.0 μl TE

Pass over G50 biotin column

Extract 2× water saturated Butanol

Add 5.00 μl 3M NaOAC, Ppt. w/ 110 μl EtOH

Resuspend DNA in 10.00 VI H2O

III. Subtraction:

A. Hybridization:

Mix:

1.00 μl Tester DNA

1.00 μl Adaptor DNA

5.00 μl 2×hybe buffer (20 mM EPPS, 2 mM EDTA)

1.00 μl H2O

Overlay with mineral oil

95° C./2 min.

Add 2.00 μl 5 M NaCl

Cool from 95° C. to 40° C. over 1 hr., incubate 40° C./16 hrs.

B. Capture:

1. Wash M270 Dynabeads.

50.0 μl Dynabeads

wash 2×200 μl lx Binding Buffer (10 mM Tris, 1 mM EDTA, 1 M NaCl; pH 8.0)

Resuspend Beads in 50 μl 1×BB

2. Prepare DNA

Add 10.0 μl 2×BB to DNA, mix well.

3. Bind DNA to Dynabeads

Mix DNA and washed Dynabeads.

37° C./1 Hrs w/ occasional mixing.

Capture beads—retain S/N=SN3

Wash beads 1× 70 μl TE, retain S/N and pool with SN3

Add 14.0 μl 3 M NaOAC

Phenol extract

Add 1.00 μl Glycogen

Ppt. DNA w/ 300 μl EtOH

Resuspend DNA in 20.0 μl 10 mM EDTA

IV. PCR Amplification

### Example 27

### Collecting and Analyzing Data from a Regulome Array

The conditions under which hybridization of labeled functional site enriched populations to a microarray containing functional sites or a combination of functional and non-functional sites is described in Example 4. In order to collect robust data the following composite experiment was performed. Four identical microarrays containing a combination of functional site sequences (positive controls), non-functional site sequences (negative controls) and sequences of undetermined functionality were constructed according to the methods described in the examples above. A functional site-enriched sample was prepared from k562 erythroleukemia cells according to Example 12 and divided into two aliquots. One aliquot was labeled according to Example 4 with Cy3 and the other was labeled according to Example 4 with Cy5. and labeled according to Example 12. A control genomic DNA sample was prepared from k562 erythroleukemia cells according to the method of Example 14 and divided into two aliquots. One aliquot was labeled according to Example 4 with Cy3 and the other was labeled according to Example 4 with Cy5. Each labeled sample was hybridized independently to one of the four aforementioned arrays according to Example 4. Following data collection and primary signal processing as described in Example 4, the two test samples (Cy3 and Cy5 labeled) were normalized to one another to exclude artifacts introduced by the differential brightness of the dyes. The same procedure was performed on the two control (Cy3 and Cy5 labeled) samples. Next, the Cy3-labeled test and control pairs were normalized to one another, and the Cy5-labeled test and control pairs were normalized to one another. Following this, the results were further analyzed to remove high-intensity (false positive) spots by filtering the data according to the ScanMer score of each spot as described above. Following these operations, the array positional intensity scores were correlated with the known positions of positive and negative controls to verify the success of the experiment. Furthermore, the array positional intensity scores from previously undetermined positions were collected to reveal which nucleic acid sequences corresponded with functional sites in the k562 erythroleukemia cell sample.

### Example 28

### Correlation of Scanmer Scores with Genomic Hybridization Signal Intensity

Following the collection of data as described in Example 27 above, trimmed correlations were computed by the standardized sums and differences method. Each variable is divided by a trimmed standard deviation. For each pair of variables, v(s) is the trimmed variance of the sum of the standardized variables and v(d) is the trimmed variance of the difference of the standardized variables. The correlation is then (v(s)−v(d))/(v(s)+v(d)). Trimmed variances (and standard deviations) are calculated by omitting the N*trim smallest and largest points. If N*trim is not an integer, it is not rounded; instead weighted sums are used (See Gnanadesikan and Kettenring, Biometrics 28, 81-124 (1972), Huber, P. J., Robust Statistics, pp. 202-203, Wiley (1981), or Gnanadesikan, R., Methods for Statistical Data Analysis of Multiple Observations, p.132, Wiley (1977), for more details). The results are depicted in FIG. 12.

Other embodiments and uses of the invention will be apparent to those skilled in the art from consideration of the specification and practice of the invention disclosed herein. All references cited herein, including all U.S. and foreign patents and patent applications including U.S. Provisional patent No. 60/108,206, U.S. patent application Ser. Nos. 09/432,576 and 10/319,440 and PCT application No. PCT/US02/15032 are specifically and entirely hereby incorporated herein by reference. It is intended that the specification and examples be considered exemplary only, with the true scope and spirit of the invention indicated by the following claims.

What is claimed is:

1. A method of profiling the genomic regulatory regions of a biological sample, comprising:
(1) contacting a sample of nucleic acid from a biological sample, with a positionally addressable array of polynucleotides under conditions such that hybridization can occur, said sample of nucleic acid being enriched in ACEs or fragments thereof of at least 10 base pairs; and
(2) detecting loci on the array where hybridization occurs,
wherein said ACEs are each a nucleotide sequence characterized as being hypersensitive to a DNA modifying agent relative to a nearby region when present in chromatin isolated from one or more cells, has a size in the range of 80-250 base pairs, and is bound by one or more sequence-specific DNA binding factors when present in chromatin isolated from one or more cells,
and wherein said array of polynucleotides comprises a plurality of polynucleotides, each affixed to a substrate, said plurality comprising different polynucleotides differing in nucleotide sequence and being situated at distinct loci of the array, said different polynucleotides being complementary and hybridizable to genomic DNA of said biological sample,
thereby profiling the genomic regulatory regions of the biological sample.

2. The method of claim 1, wherein said plurality of polynucleotides is at least 500 different polynucleotides, at least 1,000 different polynucleotides, at least 5,000 different polynucleotides, at least 10,000 different polynucleotides, or at least 20,000 different polynucleotides.

3. The method of claim 1, wherein each said ACE is further characterized as having one or more of the following characteristics:
(1) an intrinsic ability to confer hypersensitivity to the DNA modifying agent when excised from its native location and inserted into at least one different location in the genome of a cell of the same cell type;
(2) 10-50 times greater hypersensitivity to the DNA modifying agent relative to the nearby region;
(3) 50-100 times greater hypersensitivity to the DNA modifying agent relative to the nearby region;
(4) 100-150 times greater hypersensitivity to the DNA modifying agent relative to the nearby region;
(5) 150-200 times greater hypersensitivity to the DNA modifying agent relative to the nearby region;
(6) the ability to reconstitute a site that is hypersensitive to the DNA modifying agent when a nucleic acid comprising the nucleotide sequence flanked by at least 1000 bp on each side is assembled into chromatin in an in vitro reconstitution assay in the presence of nucleosomal proteins and a cell extract;
(7) is non-nucleosomal when present in chromatin isolated from one or more cells;
(8) is embedded in DNA associated with histones that have a high degree of acetylation when present in chromatin isolated from one or more cells;
(9) greater solubility than nucleosomal material in moderate salt solutions (e.g., 150 mM NaCl and 3 mM MgCl₂) when present in chromatin isolated from one or more cells;
(10) is a non-coding sequence; or
(11) does not occur greater than 10 times in a genome of the organism in which the ACE is identified.

4. A positionally addressable polynucleotide array comprising a plurality of different polynucleotides, each different polynucleotide (a) differing in nucleotide sequence, (b) being affixed to a substrate at a different locus, (c) being in the range of 10-1000 nucleotides in length, and (d) being complementary and hybridizable to a predetermined ACE, each said ACE being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and
wherein the loci at which said different polynucleotides are situated are at least 15% of the total loci of the array.

5. The positionally addressable polynucleotide array of claim 4 in which each different polynucleotide is greater than 30 nucleotides and is designed so as not to contain a sequence of in the range of 15-30 nucleotides that occurs in the genome of the organism from which the ACEs are identified greater than 10 times.

6. The positionally addressable polynucleotide array of claim 5, wherein each said different polynucleotide is designed by a method comprising
(a) identifying by comparing to an indexed polynucleotide set a sequence in said different polynucleotide, wherein said sequence consists of a nucleotide sequence in the range of 10-15 nucleotides and has a frequency count less than 11 in the genome of said organism, and wherein said indexed polynucleotide set contains binary encoded nucleotide sequences of sizes in the range of 10-15 nucleotides;
(b) determining the genomic locations of said sequence from said indexed polynucleotide set;
(c) adding prefix and suffix nucleotide sequences to said sequence according to the genomic sequence at each of said genomic locations to generate a set of candidate polynucleotides; and
(d) accepting a polynucleotide from said set of candidate polynucleotides if the respective alignment of the sequences of its added prefix and suffix sequences and the prefix and suffix sequences of said sequence in the corresponding predetermined ACE is above a given threshold.

7. A positionally addressable polynucleotide array to which nucleic acids are hybridized, said array comprising a plurality of different polynucleotides, each different polynucleotide (a) differing in nucleotide sequence and (b) being affixed at a different locus to a substrate, said nucleic acids being enriched in ACEs or fragments thereof of at least 10 base pairs, each said ACE being a nucleotide sequence characterized as being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, said nucleic acids being hybridized to one or more discrete loci on the array.

8. A positionally addressable polynucleotide array to which nucleic acids are hybridized, said array comprising a plurality of different polynucleotides, each different polynucleotide (a) differing in nucleotide sequence, (b) being affixed at a different locus to a substrate, (c) being in the range of 10-1000 nucleotides in length, and (d) being complementary and hybridizable to a predetermined ACE, each said ACE being a nucleotide sequence characterized as being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells, and
wherein the loci at which said different polynucleotides are situated are at least 15% of the total loci of the array.

9. A positionally addressable polynucleotide array to which nucleic acids are hybridized, said array comprising a plurality of different polynucleotides, each different polynucleotide (a) differing in nucleotide sequence, (b) being affixed at a different locus to a substrate, (c) being in the range of 10-1000 nucleotides in length, and (d) being complementary and hybridizable to a predetermined ACE, each said ACE being a nucleotide sequence characterized as said ACE being a nucleotide sequence characterized as being hypersensitive to a DNA modifying agent relative to a nearby region when present in chromatin isolated from one or more cells, has a size in the range of 80-250 base pairs, and is bound by one ore more sequence-specific DNA binding factors when present in chromatin isolated from one or more cells,
wherein the loci at which said different polynucleotides are situated are at least 15% of the total loci of the array;
and wherein said nucleic acids are enriched in ACEs or fragments thereof of at least 10 base pairs.

10. The positionally addressable polynucleotide array of claim 4, 7, 8, or 9, wherein said plurality of polynucleotides is at least 500 different polynucleotides, at least 1,000 different polynucleotides, at least 5,000 different polynucleotides, at least 10,000 different polynucleotides, or at least 20,000 different polynucleotides.

11. The positionally addressable polynucleotide array of claim 4, 7, 8, or 9, wherein each said ACE is further characterized as having one or more of the following characteristics:
(1) an intrinsic ability to confer hypersensitivity to the DNA modifying agent when excised from its native location and inserted into at least one different location in the genome of a cell of the same cell type;
(2) 10-50 times greater hypersensitivity to the DNA modifying agent relative to the nearby region;
(3) 50-100 times greater hypersensitivity to the DNA modifying agent relative to the nearby region;
(4) 100-150 times greater hypersensitivity to the DNA modifying agent relative to the nearby region;
(5) 150-200 times greater hypersensitivity to the DNA modifying agent relative to the nearby region;
(6) the ability to reconstitute a site that is hypersensitive to the DNA modifying agent when a nucleic acid comprising the nucleotide sequence flanked by at least 1000 bp on each side is assembled into chromatin in an in vitro reconstitution assay in the presence of nucleosomal proteins and a cell extract;
(7) is non-nucleosomal when present in chromatin isolated from one or more cells;
(8) is embedded in DNA associated with histones that have a high degree of acetylation when present in chromatin isolated from one or more cells;
(9) greater solubility than nucleosomal material in moderate salt solutions (e.g., 150 mM NaCl and 3 mM MgCl₂) when present in chromatin isolated from one or more cells;
(10) is a non-coding sequence; or
(11) does not occur greater than 10 times in a genome of the organism in which the ACE is identified.
